SEQUENCE LISTING (78 sequences)

SEQ ID NO: 1 - protein, 162 aa, Bos taurus
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 2 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 3 - protein, 162 aa, Bos taurus
LIVTQTMKGLDIQKVAGTWYSLAMAASDISLLDAQSAPLRVYVEELKPTPEGDLEILLHKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 4 - protein, 167 aa, Bos taurus
EAEAMLIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 5 - protein, 165 aa, Bos taurus
EAMLIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 6 - protein, 163 aa, Bos taurus
MLIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 7 - protein, 163 aa, Bos taurus
RLIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 8 - protein, 161 aa, Bos taurus
IVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 9 - protein, 160 aa, Bos taurus
VTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 10 - protein, 159 aa, Bos taurus
TQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 11 - protein, 158 aa, Bos taurus
QTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 12 - protein, 158 aa, Bos taurus
  MOD_RES (1)..(1): PYRROLIDONE CARBOXYLIC ACID
ETMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 13 - protein, 157 aa, Bos taurus
TMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 14 - protein, 156 aa, Bos taurus
MKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 15 - protein, 155 aa, Bos taurus
KGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 16 - protein, 154 aa, Bos taurus
GLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 17 - protein, 153 aa, Bos taurus
LDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 18 - protein, 152 aa, Bos taurus
DIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 19 - protein, 167 aa, Bos taurus; identical to SEQ ID NO: 4

SEQ ID NO: 20 - protein, 165 aa, Bos taurus; identical to SEQ ID NO: 5

SEQ ID NO: 21 - protein, 163 aa, Bos taurus; identical to SEQ ID NO: 6

SEQ ID NO: 22 - protein, 163 aa, Bos taurus; identical to SEQ ID NO: 7

SEQ ID NO: 23 - protein, 161 aa, Bos taurus
IVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 24 - protein, 160 aa, Bos taurus
VTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 25 - protein, 159 aa, Bos taurus
TQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 26 - protein, 158 aa, Bos taurus
QTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 27 - protein, 157 aa, Bos taurus
TMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 28 - protein, 156 aa, Bos taurus
MKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 29 - protein, 155 aa, Bos taurus
KGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 30 - protein, 154 aa, Bos taurus
GLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 31 - protein, 178 aa, Bos taurus
  SIGNAL (1)..(16)
MKCLLLALALTCGAQALIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 32 - protein, 178 aa, Bos taurus
  SIGNAL (1)..(16)
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 33 - protein, 178 aa, Bos taurus
  SIGNAL (1)..(16)
MKCLLLALALTCGAQALIVTQTMKGLDIQKVAGTWYSLAMAASDISLLDAQSAPLRVYVEELKPTPEGDLEILLHKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 34 - protein, 162 aa, Ovis aries
IIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 35 - protein, 180 aa, Ovis aries
  SIGNAL (1)..(18)
MKCLLLALGLALACGVQAIIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 36 - protein, 162 aa, Capra hircus
IIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAFNPTQLEGQCHV

SEQ ID NO: 37 - protein, 180 aa, Capra hircus
  SIGNAL (1)..(18)
MKCLLLALGLALACGIQAIIVTQTMKGLDIQKVAGTWYSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDKEALEKFDKALKALPMHIRLAFNPTQLEGQCHV

SEQ ID NO: 38 - protein, 162 aa, Bubalus bubalis
IIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 39 - protein, 180 aa, Bubalus bubalis
  SIGNAL (1)..(18)
MKCLLLALGLALACAAQAIIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHV

SEQ ID NO: 40 - protein, 162 aa, Equus caballus
TNIPQTMQDLDLQEVAGKWHSVAMAASDISLLDSESAPLRVYIEKLRPTPEDNLEIILREGENKGCAEKKIFAEKTESPAEFKINYLDEDTVFALDTDYKNYLFLCMKNAATPGQSLVCQYLARTQMVDEEIMEKFRRALQPLPGRVQIVPDLTRMAERCRI

SEQ ID NO: 41 - protein, 180 aa, Equus caballus
  SIGNAL (1)..(18)
MKCLLLALGLALMCGIQATNIPQTMQDLDLQEVAGKWHSVAMAASDISLLDSESAPLRVYIEKLRPTPEDNLEIILREGENKGCAEKKIFAEKTESPAEFKINYLDEDTVFALDTDYKNYLFLCMKNAATPGQSLVCQYLARTQMVDEEIMEKFRRALQPLPGRVQIVPDLTRMAERCRI

SEQ ID NO: 42 - protein, 163 aa, Equus caballus
TDIPQTMQDLDLQEVAGRWHSVAMVASDISLLDSESVPLRVYVEELRPTPEGNLEIILREGANHACVERNIVAQKTEDPAVFTVNYQGERKISVLDTDYAHYMFFCVGPPLPSAEHGMVCQYLARTQKVDEEVMEKFSRALQPLPGRVQIVQDPSGGQERCGF

SEQ ID NO: 43 - protein, 181 aa, Equus caballus
  SIGNAL (1)..(18)
MKCLLLALGLSLMCGNQATDIPQTMQDLDLQEVAGRWHSVAMVASDISLLDSESVPLRVYVEELRPTPEGNLEIILREGANHACVERNIVAQKTEDPAVFTVNYQGERKISVLDTDYAHYMFFCVGPPLPSAEHGMVCQYLARTQKVDEEVMEKFSRALQPLPGRVQIVQDPSGGQERCGF

SEQ ID NO: 44 - protein, 162 aa, Equus asinus
TNIPQTMQDLDLQEVAGKWHSVAMAASDISLLDSEEAPLRVYIEKLRPTPEDNLEIILREGENKGCAEKKIFAEKTESPAEFKINYLDEDTVFALDSDYKNYLFLCMKNAATPGQSLVCQYLARTQMVDEEIMEKFRRALQPLPGRVQIVPDLTRMAERCRI

SEQ ID NO: 45 - protein, 163 aa, Equus asinus; identical to SEQ ID NO: 42

SEQ ID NO: 46 - protein, 181 aa, Equus asinus
  SIGNAL (1)..(18)
MKCLLLALGLSLMCGNQATDIPQTMQDLDLQEVAGRWHSVAMVASDISLLDSESAPLRVYVEELRPTPEGNLEIILREGANHVCVERNIVAQKTEDPAVFTVNYQGERKISVLDTDYAHYMFFCVGPPLPSAEHGTVCQYLARTQKVDEEVMEKFSRALQPLPGHVQIIQDPSGGQERCGF

SEQ ID NO: 47 - protein, 162 aa, Rangifer tarandus
IIVTQTMKDLDVQKVAGTWYSLAMAASDISLLDAQSAPLRVYVEELKPTPGGDLEILLQKWENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRV

SEQ ID NO: 48 - protein, 180 aa, Rangifer tarandus
  SIGNAL (1)..(18)
MKCLLITLGLALACGAQAIIVTQTMKDLDVQKVAGTWYSLAMAASDISLLDAQSAPLRVYVEELKPTPGGDLEILLQKWENGKCAQKKIIAEKTEIPAVFKIDALNENKVLVLDTDYKKYLLFCMENSAEPEQSLACQCLVRTPEVDDEAMEKFDKALKALPMHIRLSFNPTQLEEQCRV

SEQ ID NO: 49 - DNA, 762 bp, Artificial Sequence
  Synthetic DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacgatgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggtttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaa

SEQ ID NO: 50 - DNA, 762 bp, Artificial Sequence
  Synthetic DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaa

SEQ ID NO: 51 - DNA, 4313 bp, Artificial Sequence
  Synthetic DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacgatgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggtttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 52 - DNA, 4313 bp, Artificial Sequence
  Synthetic DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 53 - DNA, 9273 bp, Artificial Sequence
  Synthetic DNA
tcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgtctagattacgccagctggcgaaagggggatgtgctgcaaggcgattaagttgggtaacgccagggttttcccagtcacgacgtgtacccgataaaagcggcttcctgacaggaggccgttttgttttgcagcccaccttgtaaaacgacggccagtgaatttgcggccgctcgaggattgcctgaacattgacattcggcgtccggccgggaccaccgcggactcgaagctgcctgtgctggtctggatctttggcggaggctttgaacttggttcaaaggcgatgtatgatggtacaacgatggtatcatcgtcgatagacaagaacatgcctatcgtgtttgtagcaatgaattatcgcgtgggaggtttcgggttcttgcccggaaaggagatcctggaggacgggtccgcgaacctagggctcctggaccaacgccttgccctgcagtgggttgccgacaacatcgaggcctttggtggagatccggacaaggtgacgatttggggagaatcagcaggagccatttccgtttttgatcagatgatcttgtacgacggaaacatcacttacaaggataagcccttgttccggggggccatcatggactccggtagtgttgttcccgcagaccccgtcgatggggtcaagggacagcaagtatatgatgcggtagtggaatctgcaggctgttcctcttctaacgacaccctagcttgtctgcgtgaactagactacaccgacttcctcaatgcggcaaactccgtgccaggcattttaagctaccattctgtggcgttatcatatgtgcctcgaccggacgggacggcgttgtcggcatcaccggacgttttgggcaaagcagggaaatatgctcgggtcccgttcatcgtgggcgaccaagaggatgaggggaccttattcgccttgtttcagtccaacattacgacgatcgacgaggtggtcgactacctggcctcatacttcttctatgacgctagccgagagcagcttgaagaactagtggccctgtacccagacaccaccacgtacggctctccgttcaggacaggcgcggccaacaactggtatccgcaatttaagcgattggccgccattctcggcgacttggtcttcaccattacccggcgggcattcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgagcttccgctccctccttgctctctccggtctggtctgcaccggtctggccaacgtcatctccaagcgtgccaccctggacagctggctatccaacgaggccactgttgctcgcactgccatcctcaacaacatcggtgccgatggtgcctgggtatctggtgccgacagcggtatcgttgttgcttctccctccaccgacaaccccgactacttctacacctggactcgtgtctccggtctggtcctcaagaccctcgtcgatctgttccgcaacggtgacacctcgctactctccaccattgagaactacatctccgctcaggccattgtgcagggtatctccaacccctccggtgatctgagctctggtgctggtcttggtgagcccaagttcaacgtcgatgagactgcctacaccggcagctggggtcgtccccagcgtgatggccctgctctccgtgccactgccatgatcggtttcggccagtggttgcttgacaacggctacaccagcactgccaccgacatcgtctggcctctggtccgcaacgacctctcctacgtggctcagtactggaaccagactggctacgacctctgggaggaggtcaacggcagctctttcttcaccattgccgtccagcaccgtgctctcgtcgagggctctgctttcgccactgctgtcggttcctcctgctcttggtgcgactcccaggctcctgagatcctgtgctacctgcagtcgttctggactggctctttcatcctggccaacttcgacagctctcgctctggcaaggatgccaacaccctcctcggttccatccacaccttcgaccccgaggctgcttgcgatgactccaccttccagccttgctctcctcgtgctcttgccaaccacaaggaggttgttgactccttccgctccatctacaccctgaacgacggtctgtccgactctgaggctgttgctgtcggtcgttaccccgaggacacctactacaacggcaacccctggttcctttgcaccctggctgctgctgagcagctctacgatgccctctaccagtgggacaagcagggctctctggaggtcaccgatgtctctcttgacttcttcaaggccctctactctgatgctgccactggtacctacagcagcagcagcagcacctactcctccatcgtcgatgccgtcaagaccttcgccgatggattcgtcagcattgttgaaacccacgctgcctccaacggcagcatgagccagcaatacgacaagtcggatggtgaacaattgtctgctcgtgatctcacctggtcctacgctgctctgttgactgccaacaaccgccgcaactccgttgttcctgccagctggggtgagacttccgcctcctccgttcctggtacctgcgctgccacctccgccatcggtacctactcctccgtcaccgtcacctcctggccctccattgttgccactggtggtaccaccaccactgccacccccactggctctggcagcgtgacctccacctccaagcgtgaggctttgattgtcacccagaccatgaagggtctggacatccagaaggttgctggcacctggtactctcttgccatggccgcctccgacatctccctcctcgatgcccagtccgctcctctccgtgtctacgtggaagaattgaagcccactcccgagggtgatcttgagatcctgctccagaagtgggagaacgacgagtgcgctcagaagaagatcattgcggaaaagaccaagatccctgctgttttcaagatcgatgccctcaacgagaacaaggtcctggtccttgacaccgactacaagaagtacctcctcttctgcatggagaactctgctgagcctgagcagtccctggtctgccagtgcctggtccgcacccccgaggttgatgacgaggctctcgagaagttcgacaaggccctcaaggccctgcccatgcacatccgtctgagcttcaaccccactcagctcgaggagcagtgccatatttaaaattaattaacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtacttccatagccgaggtccacagtggagcagcaacattccccatcattgctttccccaggggcctcccaacgactaaatcaagagtatatctctaccgtccaatagatcgtcttcgcttcaaaatctttgacaactccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtgtctgcacgcaaacccaacttcctcttcattctcacggatgatcaggatctccggatcaatactccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtgtaaccacgcacattgacattgcacctacactgttcgagttggctggggttcccttgcgagaggactttgacgggactccgatgcccgtgtcgactagcaagaagacccagtcaatcttgcatgagcatgtcacagtcgaatagtggggtcacgcggtgcttgagggcgaatacggctccatcggtgagtaacctctctcttactaccacggaaacatcactgacgtaaccaggacgcggccgcaagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgt

SEQ ID NO: 54 - DNA, 2046 bp, Artificial Sequence
  Synthetic DNA
atgagcttccgctccctccttgctctctccggtctggtctgcaccggtctggccaacgtcatctccaagcgtgccaccctggacagctggctatccaacgaggccactgttgctcgcactgccatcctcaacaacatcggtgccgatggtgcctgggtatctggtgccgacagcggtatcgttgttgcttctccctccaccgacaaccccgactacttctacacctggactcgtgtctccggtctggtcctcaagaccctcgtcgatctgttccgcaacggtgacacctcgctactctccaccattgagaactacatctccgctcaggccattgtgcagggtatctccaacccctccggtgatctgagctctggtgctggtcttggtgagcccaagttcaacgtcgatgagactgcctacaccggcagctggggtcgtccccagcgtgatggccctgctctccgtgccactgccatgatcggtttcggccagtggttgcttgacaacggctacaccagcactgccaccgacatcgtctggcctctggtccgcaacgacctctcctacgtggctcagtactggaaccagactggctacgacctctgggaggaggtcaacggcagctctttcttcaccattgccgtccagcaccgtgctctcgtcgagggctctgctttcgccactgctgtcggttcctcctgctcttggtgcgactcccaggctcctgagatcctgtgctacctgcagtcgttctggactggctctttcatcctggccaacttcgacagctctcgctctggcaaggatgccaacaccctcctcggttccatccacaccttcgaccccgaggctgcttgcgatgactccaccttccagccttgctctcctcgtgctcttgccaaccacaaggaggttgttgactccttccgctccatctacaccctgaacgacggtctgtccgactctgaggctgttgctgtcggtcgttaccccgaggacacctactacaacggcaacccctggttcctttgcaccctggctgctgctgagcagctctacgatgccctctaccagtgggacaagcagggctctctggaggtcaccgatgtctctcttgacttcttcaaggccctctactctgatgctgccactggtacctacagcagcagcagcagcacctactcctccatcgtcgatgccgtcaagaccttcgccgatggattcgtcagcattgttgaaacccacgctgcctccaacggcagcatgagccagcaatacgacaagtcggatggtgaacaattgtctgctcgtgatctcacctggtcctacgctgctctgttgactgccaacaaccgccgcaactccgttgttcctgccagctggggtgagacttccgcctcctccgttcctggtacctgcgctgccacctccgccatcggtacctactcctccgtcaccgtcacctcctggccctccattgttgccactggtggtaccaccaccactgccacccccactggctctggcagcgtgacctccacctccaagcgtgaggctttgattgtcacccagaccatgaagggtctggacatccagaaggttgctggcacctggtactctcttgccatggccgcctccgacatctccctcctcgatgcccagtccgctcctctccgtgtctacgtggaagaattgaagcccactcccgagggtgatcttgagatcctgctccagaagtgggagaacgacgagtgcgctcagaagaagatcattgcggaaaagaccaagatccctgctgttttcaagatcgatgccctcaacgagaacaaggtcctggtccttgacaccgactacaagaagtacctcctcttctgcatggagaactctgctgagcctgagcagtccctggtctgccagtgcctggtccgcacccccgaggttgatgacgaggctctcgagaagttcgacaaggccctcaaggccctgcccatgcacatccgtctgagcttcaaccccactcagctcgaggagcagtgccatatttaa

SEQ ID NO: 55 - DNA, 9273 bp, Artificial Sequence
  Synthetic DNA
tcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgtctagattacgccagctggcgaaagggggatgtgctgcaaggcgattaagttgggtaacgccagggttttcccagtcacgacgtgtacccgataaaagcggcttcctgacaggaggccgttttgttttgcagcccaccttgtaaaacgacggccagtgaatttgcggccgctcgaggattgcctgaacattgacattcggcgtccggccgggaccaccgcggactcgaagctgcctgtgctggtctggatctttggcggaggctttgaacttggttcaaaggcgatgtatgatggtacaacgatggtatcatcgtcgatagacaagaacatgcctatcgtgtttgtagcaatgaattatcgcgtgggaggtttcgggttcttgcccggaaaggagatcctggaggacgggtccgcgaacctagggctcctggaccaacgccttgccctgcagtgggttgccgacaacatcgaggcctttggtggagatccggacaaggtgacgatttggggagaatcagcaggagccatttccgtttttgatcagatgatcttgtacgacggaaacatcacttacaaggataagcccttgttccggggggccatcatggactccggtagtgttgttcccgcagaccccgtcgatggggtcaagggacagcaagtatatgatgcggtagtggaatctgcaggctgttcctcttctaacgacaccctagcttgtctgcgtgaactagactacaccgacttcctcaatgcggcaaactccgtgccaggcattttaagctaccattctgtggcgttatcatatgtgcctcgaccggacgggacggcgttgtcggcatcaccggacgttttgggcaaagcagggaaatatgctcgggtcccgttcatcgtgggcgaccaagaggatgaggggaccttattcgccttgtttcagtccaacattacgacgatcgacgaggtggtcgactacctggcctcatacttcttctatgacgctagccgagagcagcttgaagaactagtggccctgtacccagacaccaccacgtacggctctccgttcaggacaggcgcggccaacaactggtatccgcaatttaagcgattggccgccattctcggcgacttggtcttcaccattacccggcgggcattcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgagcttccgctccctccttgctctctccggtctggtctgcaccggtctggccaacgtcatctccaagcgtgccaccctggacagctggctatccaacgaggccactgttgctcgcactgccatcctcaacaacatcggtgccgatggtgcctgggtatctggtgccgacagcggtatcgttgttgcttctccctccaccgacaaccccgactacttctacacctggactcgtgtctccggtctggtcctcaagaccctcgtcgatctgttccgcaacggtgacacctcgctactctccaccattgagaactacatctccgctcaggccattgtgcagggtatctccaacccctccggtgatctgagctctggtgctggtcttggtgagcccaagttcaacgtcgatgagactgcctacaccggcagctggggtcgtccccagcgtgatggccctgctctccgtgccactgccatgatcggtttcggccagtggttgcttgacaacggctacaccagcactgccaccgacatcgtctggcctctggtccgcaacgacctctcctacgtggctcagtactggaaccagactggctacgacctctgggaggaggtcaacggcagctctttcttcaccattgccgtccagcaccgtgctctcgtcgagggctctgctttcgccactgctgtcggttcctcctgctcttggtgcgactcccaggctcctgagatcctgtgctacctgcagtcgttctggactggctctttcatcctggccaacttcgacagctctcgctctggcaaggatgccaacaccctcctcggttccatccacaccttcgaccccgaggctgcttgcgatgactccaccttccagccttgctctcctcgtgctcttgccaaccacaaggaggttgttgactccttccgctccatctacaccctgaacgacggtctgtccgactctgaggctgttgctgtcggtcgttaccccgaggacacctactacaacggcaacccctggttcctttgcaccctggctgctgctgagcagctctacgatgccctctaccagtgggacaagcagggctctctggaggtcaccgatgtctctcttgacttcttcaaggccctctactctgatgctgccactggtacctacagcagcagcagcagcacctactcctccatcgtcgatgccgtcaagaccttcgccgatggattcgtcagcattgttgaaacccacgctgcctccaacggcagcatgagccagcaatacgacaagtcggatggtgaacaattgtctgctcgtgatctcacctggtcctacgctgctctgttgactgccaacaaccgccgcaactccgttgttcctgccagctggggtgagacttccgcctcctccgttcctggtacctgcgctgccacctccgccatcggtacctactcctccgtcaccgtcacctcctggccctccattgttgccactggtggtaccaccaccactgccacccccactggctctggcagcgtgacctccacctccaagcgtgaggctttgattgtcacccagaccatgaagggtcttgacatccagaaggttgctggcacctggtactctcttgccatggctgcctccgacatctccctgttggatgcccagtccgctcctctgcgtgtctacgtggaagaactcaagcccactcccgagggtgaccttgagatcctcctccagaagtgggagaacggcgagtgcgctcagaagaagatcattgcggaaaagaccaagatccccgccgttttcaagatcgatgccctcaacgagaacaaggttctcgtccttgacaccgactacaagaagtacctcctcttctgcatggagaactctgctgagcctgagcagtccctggcctgccagtgcctggtccgcactcctgaagtcgatgacgaggctctcgagaagttcgacaaggccctgaaggccctccccatgcacatccgtctgagcttcaaccccactcagctcgaggagcagtgccacatctaaaattaattaacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtacttccatagccgaggtccacagtggagcagcaacattccccatcattgctttccccaggggcctcccaacgactaaatcaagagtatatctctaccgtccaatagatcgtcttcgcttcaaaatctttgacaactccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtgtctgcacgcaaacccaacttcctcttcattctcacggatgatcaggatctccggatcaatactccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtgtaaccacgcacattgacattgcacctacactgttcgagttggctggggttcccttgcgagaggactttgacgggactccgatgcccgtgtcgactagcaagaagacccagtcaatcttgcatgagcatgtcacagtcgaatagtggggtcacgcggtgcttgagggcgaatacggctccatcggtgagtaacctctctcttactaccacggaaacatcactgacgtaaccaggacgcggccgcaagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgt

SEQ ID NO: 56 - DNA, 2046 bp, Artificial Sequence
  Synthetic DNA
atgagcttccgctccctccttgctctctccggtctggtctgcaccggtctggccaacgtcatctccaagcgtgccaccctggacagctggctatccaacgaggccactgttgctcgcactgccatcctcaacaacatcggtgccgatggtgcctgggtatctggtgccgacagcggtatcgttgttgcttctccctccaccgacaaccccgactacttctacacctggactcgtgtctccggtctggtcctcaagaccctcgtcgatctgttccgcaacggtgacacctcgctactctccaccattgagaactacatctccgctcaggccattgtgcagggtatctccaacccctccggtgatctgagctctggtgctggtcttggtgagcccaagttcaacgtcgatgagactgcctacaccggcagctggggtcgtccccagcgtgatggccctgctctccgtgccactgccatgatcggtttcggccagtggttgcttgacaacggctacaccagcactgccaccgacatcgtctggcctctggtccgcaacgacctctcctacgtggctcagtactggaaccagactggctacgacctctgggaggaggtcaacggcagctctttcttcaccattgccgtccagcaccgtgctctcgtcgagggctctgctttcgccactgctgtcggttcctcctgctcttggtgcgactcccaggctcctgagatcctgtgctacctgcagtcgttctggactggctctttcatcctggccaacttcgacagctctcgctctggcaaggatgccaacaccctcctcggttccatccacaccttcgaccccgaggctgcttgcgatgactccaccttccagccttgctctcctcgtgctcttgccaaccacaaggaggttgttgactccttccgctccatctacaccctgaacgacggtctgtccgactctgaggctgttgctgtcggtcgttaccccgaggacacctactacaacggcaacccctggttcctttgcaccctggctgctgctgagcagctctacgatgccctctaccagtgggacaagcagggctctctggaggtcaccgatgtctctcttgacttcttcaaggccctctactctgatgctgccactggtacctacagcagcagcagcagcacctactcctccatcgtcgatgccgtcaagaccttcgccgatggattcgtcagcattgttgaaacccacgctgcctccaacggcagcatgagccagcaatacgacaagtcggatggtgaacaattgtctgctcgtgatctcacctggtcctacgctgctctgttgactgccaacaaccgccgcaactccgttgttcctgccagctggggtgagacttccgcctcctccgttcctggtacctgcgctgccacctccgccatcggtacctactcctccgtcaccgtcacctcctggccctccattgttgccactggtggtaccaccaccactgccacccccactggctctggcagcgtgacctccacctccaagcgtgaggctttgattgtcacccagaccatgaagggtcttgacatccagaaggttgctggcacctggtactctcttgccatggctgcctccgacatctccctgttggatgcccagtccgctcctctgcgtgtctacgtggaagaactcaagcccactcccgagggtgaccttgagatcctcctccagaagtgggagaacggcgagtgcgctcagaagaagatcattgcggaaaagaccaagatccccgccgttttcaagatcgatgccctcaacgagaacaaggttctcgtccttgacaccgactacaagaagtacctcctcttctgcatggagaactctgctgagcctgagcagtccctggcctgccagtgcctggtccgcactcctgaagtcgatgacgaggctctcgagaagttcgacaaggccctgaaggccctccccatgcacatccgtctgagcttcaaccccactcagctcgaggagcagtgccacatctaa

SEQ ID NO: 57 - DNA, 9564 bp, Artificial Sequence
  Synthetic DNA
  misc_feature (9516)..(9516): n is a, c, g, or t
atgcctcaatcctgggaagaactggccgctgataagcgcgcccgcctcgcaaaaaccatccctgatgaatggaaagtccagacgctgctgcggaagacagcgttattgatttcccaaagaaatcggggatcctttcagaggccgaactgaagatcacagaggcctccgctgcagagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatattggctgggttgatggctgcttcgagtgcagtctcatgctgccacaggtgactctggatggccccataccactcaacccatgcgtgcgaggtacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttaccctgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttccgggtggtcattggccgcctgtgatgccatctgccactaaatccgatcattgatccaccgcccacgagggcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatcgatgccctcctgtgcagcagcaggtactgctggatgaggagccatcggtctctgcacgcaaacccaacttcctcttcattctcacggatgatcaggatctccggatcaatactccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtgtaaccacgcacattgacattgcacctacactgttcgagttggctggggttcccttgcgagaggactttgacgggactccgatgcccgtgtcgactagcaagaagacccagtcaatcttgcatgagcatgtcacagtcgaatagtggggtcacgcggtgcttgagggcgaatacggctccatcggtgagtaacctctctcttactaccacggaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcgagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactataggcggaatttgcggccgcaagcttcactgacgtaaccaggacccggcggctcgaggattgcctgaacattgacattcggcgtccggccgggaccaccgcggactcgaagctgcctgtgctggtctggatctttggcggaggctttgaacttggttcaaaggcgatgtatgatggtacaacgatggtatcatcgtcgatagacaagaacatgcctatcgtgtttgtagcaatgaattatcgcgtgggaggtttcgggttcttgcccggaaaggagatcctggaggacgggtccgcgaacctagggctcctggaccaacgccttgccctgcagtgggttgccgacaacatcgaggcctttggtggagacccggacaaggtgacgatttggggagaatcagcaggagccatttccgtttttgatcagatgatcttgtacgacggaaacatcacttacaaggataagcccttgttccggggggccatcatggactccggtagtgttgttcccgcagaccccgtcgatggggtcaagggacagcaagtatatgatgcggtagtggaatctgcaggctgttcctcttctaacgacaccctagcttgtctgcgtgaactagactacaccgacttcctcaatgcggcaaactccgtgccaggcattttaagctaccattctgtggcgttatcatatgtgcctcgaccggacgggacggcgttgtcggcatcaccggacgttttgggcaaagcagggaaatatgctcgggtcccgttcatcgtgggcgaccaagaggatgaggggaccttattcgccttgtttcagtccaacattacgacgatcgacgaggtggtcgactacctggcctcatacttcttctatgacgctagccgagagcagcttgaagaactagtggccctgtacccagacaccaccacgtacgggtctccgttcaggacaggcgcggccaacaactggtatccgcaatttaagcgattggccgccattctcggcgacttggtcttcaccattacccggcgggcattcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgagcggctgaagtgcttcctcccttttagntgcaactgagagcctgagcttcatccccagcatcattacacctcagca

SEQ ID NO: 58 - DNA, 6327 bp, Artificial Sequence
  Synthetic DNA
ttgcccatcgaacgtacaagtactcctctgttctctccttcctttgcttttaggcaattggcattttttcctccatacaagttttttctaggcttttcttctgccccactgaaaaagatccgcagctgcctgtcttcttcacttttctccgagtgatatgcttcatccaggctgccacttttctgttcatatcttcttcaaaatgataggaatccagatactctcttgtggggtgccttttacctgtgatattcgatagcaaaaaatgggaagctcacagagtcaggctatttccacgttagtggatgtcgcagccagtgggtccttctttcacttgaaggctaccaataatagaccttcaatagtcactatacggtctgtatggtctgtacgggtgtatatggcacgatcaatatcgtaccatattctgtcctgctctgttctggtagtctgcgtaaactctgccaattatgttcttctatatccagttctgtcgcatgtcggctagattatttgtcctaagtttcgaatggctggctgttccttggcttctaatccccatatagaatttaatcggtcaagatggaaccgagcagttccgattgatcagaccgtatcgatgcaacaacaggtgatgttgaattctgaattctgtcaaagaatcacaatctatctcgatttcccttctttcttttttttcagcttagtttagatatgggcatcgaggttcccctaaattttgtttgccagctagacatagcactggattcttatactgtctaaagctcaccacgggcctggagaaaccgttaaaggatgcccccacaaagttatagggtagaactatgaaagtgatggggttaggtttttgaaaagaaatgaaattagaacttatataaggggatggattttagtatattttccaattctcttgttgagatcgtaataattgtcgttcttccaatcacatttagttacataaatcaccatcagctatcatactgaataacaagcaatcaagctaaaaagaatatcaatattaattaaaaaatgaaattctctactatattagccgcatctactgctttaatttccgttgttatggctgctccagtttctaccgaaactgacatcgacgatcttccaatatcggttccagaagaagccttgattggattcattgacttaaccggggatgaagtttccttgttgcctgttaataacggaacccacactggtattctattcttaaacaccaccatcgctgaagctgctttcgctgacaaggatgatctcgagaaaagattgattgtcactcaaaccatgaagggtttggacatccaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagactaagattccagctgttttcaagatcgacgctttgaacgaaaacaaggttttggttttggacaccgattacaagaaatacttgttgttctgtatggaaaactccgctgaaccagaacaatctttggcctgtcaatgtttggtcagaaccccagaagtcgatgacgaagctttggaaaagttcgacaaggctttgaaggctttgccaatgcacatcagattatctttcaacccaactcaattggaagaacaatgtcacatttaaagaattttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaacggatcgatgtacacaaccgactgcacccaaacgaacacaaatcttagcagttcctcatcactagaagccgaactgttgtcttcagtggggattggttcgacattttgccaattgctgtcgatgtaccctttcaaagccatgtaccttaaatcttcatccttggcaagtagattcatcgggtgtgtttgaagtaagaatatttgcttgtttttatggtatcaaaggtatatgttgtagaagacaatttccggtaatccaattgtctgtctgctcagtttagcacatgtatagtacgttgcacatagtctacaatattcagcattcagcattcagtatacagcatatggctaaatgatcacaaatgtgattgatgatttgacacgactagaaaagagaacgaaaaagggaaattccatgtcacgtgcgttggcacgtgacatggaatatcgaagaaagaaaaaaaaaacgatctcgtcctagtggaagcccagagtctggtccccccggagtcttcccaaaacaagaagctgacacatgttgacacagaacaccccacagcaaatgcaccacgctacgtagatcaggaagcttaactctagcgacctgtcgctcgccccacagaacctcacccgagaaccacacattacacgccgccagctcccactatactcatcttgcttcccttaagcgttctcacgattcgttcgctgcccttcttcaagagtcttctgattctaattctcattcgaaatcctctacagttaatgaattgcttgacatgacattcattgtctcatggttttggctttttggcttttgtcttttaaagctatatcaactttacatataaatatacgtcaaaaggggattcattaattagaaaattctctttttcaatagttgctattcattatcaatctattcaactcaattggttattattttcatctttttgtcatcctaaaccatcaacaatatttaaatatatctgttgctacattaagagttacttcagaaataacaaaaaaatcgatcaagaattaataaaaatgaaattctctactatattagccgcatctactgctttaatttccgttgttatggctgctccagtttctaccgaaactgacatcgacgatcttccaatatcggttccagaagaagccttgattggattcattgacttaaccggggatgaagtttccttgttgcctgttaataacggaacccacactggtattctattcttaaacaccaccatcgctgaagctgctttcgctgacaaggatgatctcgagaaaagattgattgtcactcaaaccatgaagggtttggacatccaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagactaagattccagctgttttcaagatcgacgctttgaacgaaaacaaggttttggttttggacaccgattacaagaaatacttgttgttctgtatggaaaactccgctgaaccagaacaatctttggcctgtcaatgtttggtcagaaccccagaagtcgatgacgaagctttggaaaagttcgacaaggctttgaaggctttgccaatgcacatcagattatctttcaacccaactcaattggaagaacaatgtcacatttaaagctgaggaagactgttacttaataaaagcaaaagaaaaaaaaagaaaaatacacacaaacacacacgatgcagacttctatcagaggaacttcgtttcacatcatctcacatttggtcttactctcttcgtctatttcattgtcttaatttagcacattttcggttctttgtcagatcaaagattctattgccaatagaaaccctttcactgcccaaaaattgatgcgttgctcgatctccgttccacttgcataggaacagaaagcctataatcagaatttcttttaacaccctagttaacgctttccggcatcttccagaccacagtatatccatccgcctcctgttgcgtatccctatctggattaacatcactgccacagatcgaattgcaagaagccacacttcacgtgatccactcgttcatcaggtttgtagcttcatggcgcaggacttctgatgatgaactatctggctcatccggatggatcacaaggatggataccctcagacagtacgtttccgtatggagcgatcttacaagaaccaacagttgatcctattactttttttttattttttgtccctccgggatggcaagagggacaaagaagaatcttcgttcttctttcttgttctcaacttcccagcttccgtgtgattaccctccgggacaacagaaaaactggcattcggtatcccgggaatctgctgagaaggaaagaaaacgaaaaaaaaattgtacatttgtgtcacattatgaattacaggaagtcagaaaacaggcagcacatgtctcgcacatgcatgtccatcagacgagacattatgagacatgcacgcgtgtgagagacatagcaaaagtctctccagtacacacagaaagacacgttcacaatccaggcaccccacagagaaaaaaaaaagaagaagcccggaagctggcacgccatcatcaaccaccgctcggtttacacgcatcccaactgtcttttttttctggaatcctataataactggcatctggaaatcacgttgtatgttgcaccatagtgactggctgtctgactagcaaacattgattccctgattcccatttggctcaattttgatgagaaacagttgattgattcttgtcaatttttttttctttggaccaccaccaaccaattgacattgaagtactttcccatgatttgaggttatataaaaggacgttcaaatcactttcaaggttaattcagttttgtcaattgatttaagttcaattgttaacaaatttaatttaattcgaaacaaaccaaaccaattcatttgaattaacaaaccaacccacaaaacaaaaaaaaatgaaattctctactatattagccgcatctactgctttaatttccgttgttatggctgctccagtttctaccgaaactgacatcgacgatcttccaatatcggttccagaagaagccttgattggattcattgacttaaccggggatgaagtttccttgttgcctgttaataacggaacccacactggtattctattcttaaacaccaccatcgctgaagctgctttcgctgacaaggatgatctcgagaaaagattgattgtcactcaaaccatgaagggtttggacatccaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagactaagattccagctgttttcaagatcgacgctttgaacgaaaacaaggttttggttttggacaccgattacaagaaatacttgttgttctgtatggaaaactccgctgaaccagaacaatctttggcctgtcaatgtttggtcagaaccccagaagtcgatgacgaagctttggaaaagttcgacaaggctttgaaggctttgccaatgcacatcagattatctttcaacccaactcaattggaagaacaatgtcacatttaaagctttgccaagcttcctccattgagtcattatcactttgtccaataaactttacactaaaaaagaaccaatcttagtaaatatatgtaaaaacgtgtatgcatatattctaattcactctatattattactataaaccagcttctcggcccttaacctacagccttgcatcgtctctgctctttactcattccttcccctgtgtactttataatcatctgatcgtagatctatatttgaaataaagaattgttaaaactgaaactcatccatctcaaaggaatgaccataaaagttgtaaagaaagcctgtatgcgaagccacaatcctttccaacagaccatactaagt

SEQ ID NO: 59 - DNA, 1000 bp, Artificial Sequence
  Synthetic DNA
aatgtattcttgtttttgaaacccattttccatccaaaggcttctactattcgtacctttaccaattacctacaggcaccgtaaccagattagatgttgttacgactttgttaaatacttactatgtgagacaactatattattaaaggctcgagactgtaagactgaataacttgcaatggcacaatatttttccttcgccccagatttatccgaggcacagcatagcggaaaaaccatagagtatccagccactaataacgccatgttacgatgaccaggccttcccatctcaaagaacttgaataaactcaatgccattcacattcgtttatcatttaatcccactgttcgtttctttccagcagaaattgagcttaatcagaagaacatggagtacatagagaacagaatgtaaggtacaagtatactagaatccatcatgtggggatacggccataaagttagccaatggactaccgttccattctcaacgtaagaccccatccaaacgagacagctgatcgaaaagaacagtgatcgtatggggtcacgttgcttttaactgtttttatggatttgcctgtaaagctctcaaatcccgtctaccttattttaatggttgatgagagaaaaggtttcgtttgctattgggcagatgtgttaccacatctgcagagcaggtgtggatgtgcaatgagcaattgtttatacaacaaacattggcaacccttcaaagggacgtacttacgtacgttgttcgctatttagggcaattctcaccttgcgttattaacccaattagggtagggccattacaactcggataactgtgcatctccagtacatgattatgattgattatatatatatatatatatatatatatggattggatggggccattggaactgaagggttcaatatatcaatatgtatgtgtcatttccttgccatctccttgacttaaagcagcacactcatcaaagcgaaataaaat

SEQ ID NO: 60 - DNA, 1000 bp, Artificial Sequence
  Synthetic DNA
tcgatacccttttgattccaattgtgtgtttccatcaattattgtgaacatgtttgaagtgtgatctcaaacattaacaaaactcacagactattttaaatacccccaacttcttctacggcccttgcaggtttccaaacaggaaaggactgtatatggctcccgaccttattggtcgcctgctttgtttttgagttcctcttatgttaaaagcttatgttatgcagttttaatttatatttaacaacagttctaaaacgaattttatcaaaactaaattgacttcttgataccatcagcgagtggttattagcagtttaccatgtagtttctatgatgctgcgctactacctttcattgtgctggaaaagcctcactagttggtttctgacaccttggaacaactgaatccttttaatggatggatctgacaataagggcagatccattgctataggtcgggtactttcttttcgacagccagtaaatgtctattatagaagttgaataagtgtttaagtactgctcttgattccagtcccagctgccttaaaatcctgacttgatgaattgatcgtagtctacaacattttaattcaattccaacttaagagactcaaacatgcagaataaaatgacttttgaagtcgtcaaaagcagctacaaaacataaactaacgaacgagactggtatttgtaatttcagtgcaacctggggaatgattactggaatcttcaatggtatcgggacttttactgtattcaaaattatggccatctggacccttgattggacgaacagagtgaagggtttatgatatgcatgaaatgtcacactcgagagcgaatcaattctatgtcaacttaacctcaattaaggtctatttctacacagtatggcgtttgaatctatctacctgcgaatcctgtatttacttggtagatggagaagctatacagcaactcattaaggataccgtgcacactgttacaagaaata

SEQ ID NO: 61 - protein, 158 aa, Bos taurus; identical to SEQ ID NO: 26
  MOD_RES (1)..(1): PYRROLIDONE CARBOXYLIC ACID

SEQ ID NO: 62 - protein, 410 aa, Komagataella phaffii
MIFDGTTMSIAIGLLSTLGIGAEAKVHSAKIHKHPVSETLKEANFGQYVSALEHKYVSLFNEQNALSKSNFMSQQDGFAVEASHDAPLTNYLNAQYFTEVSLGTPPQSFKVILDTGSSNLWVPSKDCGSLACFLHAKYDHDESSTYKKNGSSFEIRYGSGSMEGYVSQDVLQIGDLTIPKVDFAEATSEPGLAFAFGKFDGILGLAYDSISVNKIVPPIYKALELDLLDEPKFAFYLGDTDKDESDGGLATFGGVDKSKYEGKITWLPVRRKAYWEVSFDGVGLGSEYAELQKTGAAIDTGTSLIALPSGLAEILNAEIGATKGWSGQYAVDCDTRDSLPDLTLTFAGYNFTITPYDYTLEVSGSCISAFTPMDFPEPIGPLAIIGDSFLRKYYSVYDLGKDAVGLAKSI

SEQ ID NO: 63 - protein, 410 aa, Komagataella phaffii
MMFDGTTMSIAIGLLSTLGIGVEANVHSAKIHKHPVSETLKEANFGQYVSALEHKYVSLFNEQNTLSKSNFVSQQDGFAIEASHDAPLTNYLNAQYFTEVSLGTPPQSFKVILDTGSSNLWVPSKDCGSLACFLHAKYDHDESSTYKKNGSSFEIRYGSGSMEGYVSQDVLQIGDLTIPKVDFAEATSEPGLAFAFGKFDGILGLAYDSISVNKIVPPIYKALELDLLDEPKFAFYLGDTDKDESDGGLATFGGVDKSKYEGKITWLPVRRKAYWEVSFDGVGLGSEYAELQKTGAAIDTGTSLIALPSGLAEILNAEIGATKGWSGQYAVDCDTRDSLPDLTLTFAGYNFTITPYDYTLEVSGSCISAFTPMDFPEPIGPLAIIGDSFLRKYYSVYDLGNDAVGLAKSI

SEQ ID NO: 64 - DNA, 9525 bp, Artificial Sequence
  Synthetic construct
gcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctatgcggccgccaccaagtatcacagttggattaattagatatgaaaactaattgtttatttacaataagacgcagatagcccctttcgttcagaatagtcatttcatcatatttcctgatatatatgtctacattagtcttcaattgaactctgtcacagtggccgagtggttaaggcgatgccctgctattctttgaaagtagttaaggcattgggttctacctgcgcaggttcgaatcctgtctgtgacgagcatcatgcgaagcttcaagttttagagctagaaatagcaagttaaaataaggctagtccgttatcaacttgaaaaagtggcaccgagtcggtggtgctttttttgttttttatgtctcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaatcaacatctttggataatatcagaatgagaaagaacagatacgcagtacgttttttggtgagctctttgcacttctttagttctttccatcaatatcagttttttaaacttttaggactaaaagtgatgtttaacttcaaaatgtttaaaattttgttcttcccgacgttcattaagaatactaatacactttaataattattttaatattttgttctaaataatgacttttaattaaaaaagataaaatataaaaacatcataataactcaccagaggttaagaacaaaaaaacaaattagatatctgctaatccaatatagttaaatcaatctttccttggtataatgggtatattacatatatttcaaggaccgacactcctaccaaatatctaaaatttaccatattaacataacatgtatataaacgtcaaatcataatcagcactagatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacatggacaagaagtactccattgggctcgatatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatctcttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggc

SEQ ID NO: 65 - DNA, 4893 bp, Artificial Sequence
  Synthetic construct
gatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacatggacaagaagtactccattgggctcgatatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatctcttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgg

SEQ ID NO: 66 - DNA, 444 bp, Artificial Sequence
  Synthetic construct
aagtatcacagttggattaattagatatgaaaactaattgtttatttacaataagacgcagatagcccctttcgttcagaatagtcatttcatcatatttcctgatatatatgtctacattagtcttcaattgaactctgtcacagtggccgagtggttaaggcgatgccctgctattctttgaaagtagttaaggcattgggttctacctgcgcaggttcgaatcctgtctgtgacgagcatcatgcgaagcttcaagttttagagctagaaatagcaagttaaaataaggctagtccgttatcaacttgaaaaagtggcaccgagtcggtggtgctttttttgttttttatgtctcggtggagctccagcttttgttccctttagtgagggttaattgcgcgcttggcgtaatcatggtcatagctgtttcctgtgtgaaat

SEQ ID NO: 67 - DNA, 2033 bp, Artificial Sequence
  Synthetic construct
gaaatagacgcagatcgggaacactgaaaaatacacagttattattcatttaaataacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagattgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttc

SEQ ID NO: 68 - DNA, 86 bp, Artificial Sequence
  Synthetic construct
atctaaaccattttacattctagagttccatttctcaattactgataatcaatttaaagaaacatccaaagacgaaaggttgaatg

SEQ ID NO: 69 - DNA, 87 bp, Artificial Sequence
  Synthetic construct
tacatcttcactacctgataagtaaccaaataagttcagctgagcaacttttattcttgcccgcacaaacgaaggtttcacttaatc

SEQ ID NO: 70 - DNA, 2091 bp, Artificial Sequence
  Synthetic construct
atctaaaccattttacattctagagttccatttctcaattactgataatcaatttaaagaaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagattgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcgggcaagaataaaagttgctcagctgaacttatttggttacttatcaggtagtgaagatgta

SEQ ID NO: 71 - protein, 166 aa, Bos taurus
EAEALIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 72 - protein, 164 aa, Bos taurus
EALIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 73 - protein, 166 aa, Bos taurus
EAEALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 74 - protein, 164 aa, Bos taurus
EALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 75 - DNA, 759 bp, Bos taurus
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacgatgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggtttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaa

SEQ ID NO: 76 - DNA, 759 bp, Bos taurus
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaa

SEQ ID NO: 77 - DNA, 4310 bp, Artificial Sequence
  Synthetic construct
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacgatgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggtttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 78 - DNA, 4310 bp, Artificial Sequence
  Synthetic construct
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctttgattgtcactcaaaccatgaagggtttggacattcaaaaggttgccggtacctggtactccttggccatggctgcctccgatatctctctattggatgctcaatctgctccattacgtgtctacgttgaagaattgaaaccaactccagaaggtgacttggaaatcttattgcaaaaatgggaaaacggtgaatgtgctcaaaagaagatcattgctgaaaagaccaagatcccagctgttttcaagattgacgctttgaacgaaaacaaggtcttggttttggacactgactacaagaaatacttgttgttctgtatggaaaactctgctgaaccagaacaatccttggcttgtcaatgtttggtcagaaccccagaagtcgatgacgaagctttggaaaagttcgacaaggctttgaaggctttgccaatgcacatcagattatctttcaacccaactcaattggaagaacaatgtcacatctagtaaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

(19) United States
(12) Patent Application Publication
(10) Pub. No.: US 2024/0294589 A1
(43) Pub. Date: Sep. 5, 2024

(54) RECOMBINANT PROTEINS

(71) Applicant: FONTERRA CO-OPERATIVE GROUP LIMITED, Auckland (NZ)

(72) Inventors: Pieter Nelisse, Echt (NL); Cees Sagt, Echt (NL); Alrik Los, Echt (NL); Jeremy Hill, Auckland (NZ); Skelte Anema, Auckland (NZ); Sheelagh Hewitt, Auckland (NZ); Robertus Antonius Mijndert Van der Hoeven, Echt (NL); Alan Welman, Auckland (NZ); Bernard Meijrink, Echt (NL)

(21) Appl. No.: 18/571,576

(22) PCT Filed: Jun. 24, 2022
(86) PCT No.: PCT/IB2022/055858

§ 371 Date: Dec. 18, 2023

(30) Foreign Application Priority Data
Jun. 24, 2021 (AU) 2021901917
Nov. 10, 2021 (AU) 2021903589

(51) Int. Cl. C07K 14/47; A23L 29/00; C12N 15/81

(52) U.S. Cl. CPC C07K 14/4717; A23L 29/035; C12N 15/81; C07K 2319/02

(57) ABSTRACT

The invention relates to recombinant elongated β-lactoglobulin proteins, compositions comprising two or more recombinant β-lactoglobulin proteins of differing amino acid sequence, the recombinant β-lactoglobulin proteins having at least 70% sequence identity to a wild type, mature β-lactoglobulin, and wherein at least one of the β-lactoglobulin proteins is an elongated β-lactoglobulin protein, compositions comprising a plurality of recombinant β-lactoglobulin proteins heterogeneous in amino acid sequence, food products comprising the compositions, methods of making such food products, methods for producing the recombinant β-lactoglobulin proteins, and host cells and polynucleotide expression vectors for use in such methods.

### FIELD OF THE INVENTION

The present invention relates generally to recombinant proteins, and in particular to β-lactoglobulin fermentation protein ingredients. The ingredients may be used to bring about specialised functionality in a food product, either as a sole source of protein in the product, or in combination with plant or animal proteins, or other recombinant proteins such as those produced by fermentation.

More specifically, the present invention relates to recombinant, elongated β-lactoglobulin proteins, compositions comprising said proteins, and compositions comprising recombinant β-lactoglobulin proteins heterogeneous in amino acid sequence. The invention further relates to methods and tools for manufacture of the proteins and compositions, and food products comprising the proteins and compositions.

### BACKGROUND TO THE INVENTION

Plant-based products may offer the benefits of natural dairy products or composite foods where dairy protein has been replaced by plant-sourced components. However, plant-based products are frequently inferior in various functional, nutritional, and sensory attributes provided to many foods by animal-sourced components, particularly proteins including beneficial bioactive constituents found in natural bovine milk.

With the increasing popularity of plant-based foods, for the same volume of consumption, consumers will receive less nutrition, particularly protein and amino acid nutrition, as is found in similar products that contain dairy protein or other animal proteins. In addition, for some consumers wanting to make a plant-based choice in preference to products containing animal-proteins, they do not do so because such products have inferior sensory attributes, such as unfavourable flavour, odour and/or colour. Food manufacturers making such plant-based foods are also losing out on the functional benefits of dairy protein ingredients such as thickening, gelling, texture modification, heat stability and foaming attributes.

There is a need for recombinant animal proteins, including dairy proteins, for use in plant-based and animal-based foods to address the abovementioned deficit in nutrition and unfavourable functional and sensory properties associated with plant-based proteins.

It is an object of the present invention to provide improved or alternative recombinant β-lactoglobulin proteins, and/or to at least provide the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY OF THE INVENTION

In one aspect the invention relates to a composition comprising a plurality of recombinant β-lactoglobulin proteins heterogeneous in amino acid sequence, the recombinant proteins having at least 70% sequence identity to a wild type, mature β-lactoglobulin, wherein the plurality comprises at least one modified β-lactoglobulin protein comprising or consisting of an amino acid sequence that comprises
a) an N-terminal truncation of from about 1 to about 20 amino acids relative to the sequence of a wild type, mature β-lactoglobulin;
b) an N-terminal elongation of from about 1 to about 20 amino acids relative to the sequence of a wild type, mature β-lactoglobulin; or
c) one or more substitutions of from about 1 to about 20 amino acids relative to the sequence of a wild type, mature β-lactoglobulin,
   and wherein
A) the amount of modified β-lactoglobulin protein is at least 40% of the total recombinant β-lactoglobulin proteins in the composition;
B) the amount of modified β-lactoglobulin protein comprising or consisting of an amino acid sequence that comprises an N-terminal elongation of from about 1 to about 20 amino acids relative to the sequence of a wild type, mature β-lactoglobulin, is at least about 10% of the total recombinant β-lactoglobulin proteins in the composition;
C) the amount of modified β-lactoglobulin protein comprising or consisting of an amino acid sequence that comprises an N-terminal truncation of from about 1 to about 20 amino acids relative to the sequence of a wild type, mature β-lactoglobulin, is at least about 30% of the total recombinant β-lactoglobulin proteins in the composition; or
D) any combination of any two or more of (A) to (C).

In another aspect the invention relates to a composition comprising a plurality of recombinant β-lactoglobulin proteins heterogeneous in amino acid sequence, the recombinant proteins having at least 70% sequence identity to SEQ ID No: 1, wherein the plurality comprises at least one modified β-lactoglobulin protein comprising or consisting of an amino acid sequence that comprises:
a) an N-terminal truncation of from about 1 to about 20 amino acids relative to the sequence of SEQ ID No: 1;
b) an N-terminal elongation of from about 1 to about 20 amino acids relative to the sequence of SEQ ID No: 1; or
c) one or more substitutions of from about 1 to about 20 amino acids relative to the sequence of SEQ ID No: 1,
   and wherein
A) the amount of modified β-lactoglobulin protein is at least 40% of the total recombinant β-lactoglobulin proteins in the composition;
B) the amount of modified β-lactoglobulin protein comprising or consisting of an amino acid sequence that comprises an N-terminal elongation of from about 1 to about 20 amino acids relative to the sequence of SEQ ID No: 1, is at least about 10% of the total recombinant β-lactoglobulin proteins in the composition;
C) the amount of modified β-lactoglobulin protein comprising or consisting of an amino acid sequence that comprises an N-terminal truncation of from about 1 to about 20 amino acids relative to the sequence of SEQ ID No: 1, is at least about 30% of the total recombinant β-lactoglobulin proteins in the composition; or
D) any combination of any two or more of (A) to (C).

In another aspect the invention relates to a composition comprising a plurality of recombinant β-lactoglobulin proteins heterogeneous in amino acid sequence, the recombinant proteins having at least 70% sequence identity to SEQ ID No: 2, wherein the plurality comprises at least one modified β-lactoglobulin protein comprising or consisting of an amino acid sequence that comprises:
a) an N-terminal truncation of from about 1 to about 20 amino acids relative to the sequence of SEQ ID No: 2;
b) an N-terminal elongation of from about 1 to about 20 amino acids relative to the sequence of SEQ ID No: 2; or
c) one or more substitutions of from about 1 to about 20 amino acids relative to the sequence of SEQ ID No: 2,
   and wherein
A) the amount of modified β-lactoglobulin protein is at least 40% of the total recombinant β-lactoglobulin proteins in the composition;
B) the amount of modified β-lactoglobulin protein comprising or consisting of an amino acid sequence that comprises an N-terminal elongation of from about 1 to about 20 amino acids relative to the sequence of SEQ ID No: 2, is at least about 10% of the total recombinant β-lactoglobulin proteins in the composition;
C) the amount of modified β-lactoglobulin protein comprising or consisting of an amino acid sequence that comprises an N-terminal truncation of from about 1 to about 20 amino acids relative to the sequence of SEQ ID No: 2, is at least about 30% of the total recombinant β-lactoglobulin proteins in the composition; or
D) any combination of any two or more of (A) to (C).

In one aspect the invention relates to a composition comprising two or more recombinant β-lactoglobulin proteins of differing amino acid sequence, the recombinant β-lactoglobulin proteins having at least 70% sequence identity to a wild type, mature β-lactoglobulin, and wherein at least one of the β-lactoglobulin proteins is an elongated β-lactoglobulin protein. Preferably, the elongated β-lactoglobulin protein comprises an N-terminal elongation.

In another aspect the invention relates to a food product comprising a recombinant, elongated β-lactoglobulin protein having at least 70% sequence identity to a wild type, mature β-lactoglobulin. Preferably, the elongated β-lactoglobulin protein comprises an N-terminal elongation.

In another aspect the invention relates to a food product comprising a composition disclosed herein.

In a further aspect the invention relates to a method for preparing a food product, the method comprising
a) providing a composition disclosed herein, and
b) mixing the composition with one or more additional ingredients to produce the food product.

In a further aspect the invention relates to a method for preparing a food product, the method comprising
a) providing a recombinant, elongated β-lactoglobulin protein having at least 70% sequence identity to a wild type, mature β-lactoglobulin, and
b) mixing the composition with one or more additional ingredients to produce the food product.

In various embodiments the recombinant, elongated β-lactoglobulin protein comprises a protein or composition of the invention.

In one aspect the invention relates to a polynucleotide expression vector for expressing a plurality of recombinant β-lactoglobulin proteins heterogeneous in amino acid sequence, the polynucleotide expression vector encoding
a) a signal sequence,
b) a leader sequence,
c) a β-lactoglobulin protein, and
d) a processing site selected from KR, KREA, KREAEA and KREAEAM located between the leader sequence and the mature dairy protein.

In a further aspect the invention provides a polynucleotide expression vector for expressing a composition or recombinant, elongated β-lactoglobulin described herein, the polynucleotide expression vector encoding
a) a signal sequence;
b) a leader sequence;
c) a β-lactoglobulin protein; and
d) a processing site selected from KR, KREA, KREAEA and KREAEAM located between the leader sequence and the mature dairy protein.

In another aspect the invention relates to a host cell comprising an expression vector described herein.

In a further aspect the invention relates to a host cell for expressing a plurality of recombinant β-lactoglobulin proteins heterogeneous in amino acid sequence, wherein the species of the host cell is selected from Pichia pastoris, Kluyveromyces lactis and Aspergillus niger, and wherein the host cell comprises a polynucleotide encoding a) a signal sequence,
b) a leader sequence,
c) a β-lactoglobulin protein, and
d) a processing site selected from KR, KREA, KREAEA and KREAEAM located between the leader sequence and the β-lactoglobulin protein.

In a further aspect the invention relates to a host cell for expressing a plurality of recombinant β-lactoglobulin proteins heterogeneous in amino acid sequence, wherein the species of the host cell is selected from Pichia pastoris, Kluyveromyces lactis, Saccharomyces cerevisiae and Aspergillus niger, and wherein the host cell comprises a polynucleotide encoding
a) a signal sequence,
b) a leader sequence,
c) a β-lactoglobulin protein, and
d) a processing site selected from KR, KREA, KREAEA and KREAEAM located between the leader sequence and the β-lactoglobulin protein.

In another aspect the invention provides a host cell for expressing a composition or recombinant, elongated β-lactoglobulin described herein, wherein the species of the host cell is selected from Pichia pastoris, Kluyveromyces lactis, Saccharomyces cerevisiae and Aspergillus niger, and wherein the host cell comprises a polynucleotide encoding
a) a signal sequence;
b) a leader sequence;
c) a β-lactoglobulin protein; and
d) a processing site selected from KR, KREA, KREAEA and KREAEAM located between the leader sequence and the β-lactoglobulin protein.

In one aspect the invention relates to a method for producing a plurality of recombinant β-lactoglobulin proteins heterogeneous in amino acid sequence, comprising
a) culturing a host cell described herein in a culture medium under conditions sufficient to allow for expression of the one or more recombinant β-lactoglobulin proteins, and
b) isolating the one or more recombinant proteins from the culture medium.

In a further aspect the invention provides a method for producing a composition or recombinant, elongated β-lactoglobulin described herein, the method comprising
a) culturing a host cell described herein in a culture medium under conditions sufficient to allow for expression of one or more recombinant β-lactoglobulin proteins; and
b) isolating the one or more recombinant β-lactoglobulin proteins from the culture medium.

In various embodiments the recombinant β-lactoglobulin proteins have at least 70%, 75%, 80%, 85%, 90%, 95% or 99% sequence identity to any one of SEQ ID Nos: 1-3.

In various embodiments the β-lactoglobulin protein may be selected from a mature β-lactoglobulin protein or a full length β-lactoglobulin protein.

In various embodiments the polynucleotide expression vector or the polynucleotide may encode a wild type β-lactoglobulin protein. In various embodiments the polynucleotide expression vector or the polynucleotide may encode a wild type, mature β-lactoglobulin protein. In various embodiments the wild type β-lactoglobulin protein is a bovine β-lactoglobulin protein, such as a bovine wild type mature β-lactoglobulin protein.

In various embodiments the polynucleotide encodes a β-lactoglobulin protein comprising, or consisting of a sequence of any one of SEQ ID Nos:1 to 3, 34, 36, 38, 40, 42, 44, 45, or 47. In various embodiments the polynucleotide encodes a β-lactoglobulin protein comprising, or consisting of a sequence of any one of SEQ ID Nos:1 to 3.

In various embodiments the processing site may be selected from KR, KREA, and KREAEAM. In other embodiments the processing site may be selected from KREA, KREAEA and KREAEAM.

In various embodiments the host cell lacks an operative pep4 gene, or has been modified to produce less PEP4 protein than a wild type control cell. In one embodiment the host cell lacks an operative pep4 gene.

In another aspect the invention relates to a recombinant, elongated β-lactoglobulin protein having an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of
   i) 2 or more contiguous amino acids from the sequence KREAEAM; or
   ii) R.

In various embodiments the N-terminal elongation has a sequence comprising or consisting of 2 or more contiguous amino acids from the sequence
a) REAEAM
b) EAEAM; or
c) EAEA.

In various embodiments the N-terminal elongation has a sequence comprising or consisting of any one of:
a) EAEAM,
b) EAEA,
c) EAM,
d) EA; and
e) R.

In various embodiments the N-terminal elongation has a sequence comprising or consisting of EA, or two or more repeats of EA, for example three or more repeats of EA, four or more repeats of EA, or five or more repeats of EA. For example, the N-terminal elongation has a sequence comprising or consisting of EA, EAEA, EAEAEA, EAEAEAEA, or EAEAEAEAEA.

In various embodiments the recombinant, elongated β-lactoglobulin protein has an amino acid sequence comprising or consisting of a sequence of any one of SEQ ID Nos: 4, 5, 7, 19-20, 22 and 71-74.

In one aspect the invention relates to a composition comprising one or more recombinant, elongated β-lactoglobulin proteins of the invention.

The following embodiments may relate to any of the above aspects.

In various embodiments the wild type, mature β-lactoglobulin has a sequence of one of SEQ ID Nos:1 to 3, 34, 36, 38, 40, 42, 44, 45, or 47. In some embodiments the wild type, mature β-lactoglobulin has a sequence of any one of SEQ ID Nos: 1-3. In some embodiments the wild type, mature β-lactoglobulin has a sequence of any one of SEQ ID Nos: 1 or 2.

In various embodiments the amount of recombinant β-lactoglobulin protein having an amino acid sequence comprising an aspartic acid at a position equivalent to position 80 of SEQ ID No: 1 and a valine at a position equivalent to position 134 of SEQ ID No: 1, is at least 90%, 95% or at least 99% of the total recombinant β-lactoglobulin protein in the composition.

In various embodiments the amount of recombinant β-lactoglobulin protein having an amino acid sequence comprising a glycine at a position equivalent to position 80 of SEQ ID No: 2 and an alanine at a position equivalent to position 134 of SEQ ID No: 2, is at least 90%, 95% or at least 99% of the total recombinant β-lactoglobulin proteins in the composition.

In various embodiments the amount of modified β-lactoglobulin protein in the composition is at least 1, 2, 3, 4, 5, 10, 15, 20, 25, 30, 35, 40, 45, 50, 55, 60, 65, 70, 75, 80, 85, 90, 95 or 99% of the total recombinant β-lactoglobulin proteins in the composition, and various ranges may be selected from between any two of these values. For example, from about 1 to about 100, about 1 to about 99, about 1 to about 90, about 1 to about 80, about 1 to about 70, about 1 to about 60, about 1 to about 50, about 10 to about 100, about 10 to about 99, about 10 to about 90, about 10 to about 80, about 10 to about 70, about 10 to about 60, about 10 to about 50, about 20 to about 100, about 20 to about 99, about 20 to about 90, about 20 to about 80, about 20 to about 70, about 20 to about 60, about 20 to about 50, about 30 to about 100, about 30 to about 99, about 30 to about 90, about 30 to about 80, about 30 to about 70, about 30 to about 60, about 30 to about 50, about 40 to about 100, about 40 to about 99, about 40 to about 90, about 40 to about 80, about 40 to about 70, about 40 to about 60, about 40 to about 50, about 50 to about 100, about 50 to about 99, about 50 to about 90, about 50 to about 80, about 50 to about 70, about 50 to about 60, about 60 to about 100, about 60 to about 99, about 60 to about 90, about 60 to about 80, about 60 to about 70, about 70 to about 100, about 70 to about 99, about 70 to about 90, about 70 to about 80, about 80 to about 100, about 80 to about 99, or about 80 to about 90% of the total recombinant β-lactoglobulin proteins in the composition.

In various embodiments the recombinant β-lactoglobulin proteins, or the one or more recombinant, elongated β-lactoglobulin proteins, may have at least about 70, 75, 80, 85, 90, 95, 99 or 100% sequence identity to
a) a wild type, mature β-lactoglobulin;
b) any one of SEQ ID Nos:1 to 3, 34, 36, 38, 40, 42, 44, 45, or 47;
c) any one of SEQ ID Nos:1-3;
d) SEQ ID No:1 or 2;
e) SEQ ID No:1; or
f) SEQ ID No:2.

In various embodiments the N-terminal elongation consists of about 1, 2, 3, 4, 5, 6, 7, 8, 9, 10, 11, 12, 13, 14, 15, 16, 17, 18, 19 or about 20 amino acids, and various ranges may be selected from between any two of these values, for example, from about 1 to about 20, about 1 to about 10, about or 1 to about 5 amino acids.

In various embodiments the N-terminal truncation consists of about 1, 2, 3, 4, 5, 6, 7, 8, 9, 10, 11, 12, 13, 14, 15, 16, 17, 18, 19 or about 20 amino acids, and various ranges may be selected from between any two of these values, for example, from about 1 to about 20, about 1 to about 10, about or 1 to about 5 amino acids.

In various embodiments the plurality comprises at least one modified β-lactoglobulin protein comprising or consisting of an amino acid sequence that comprises one or more substitutions of about 1, 2, 3, 4, 5, 6, 7, 8, 9, 10, 11, 12, 13, 14, 15, 16, 17, 18, 19 or about 20 amino acids relative to the sequence of one of SEQ ID Nos: 1 to 3, and various ranges may be selected from between any two of these values, for example, from about 1 to 10, 1 to 5, 1 to 4, or 1 to 3 amino acids relative to the sequence of one of SEQ ID Nos: 1 to 3.

In various embodiments the one or more substitutions comprises one or more or from 1 to about 20, about 1 to about 10, about 1 to about 5, about 1 to about 3 or 1 or 2 essential amino acids.

In various embodiments the one or more essential amino acids comprise or consist of one or more histidine residues.

In various embodiments the amount of recombinant β-lactoglobulin protein comprising or consisting of an amino acid sequence of any one of a) a wild type, mature β-lactoglobulin; b) SEQ ID Nos:1 to 3, 34, 36, 38, 40, 42, 44, 45, or 47; c) SEQ ID Nos 1 to 3, or d) SEQ ID No 1 or 2, is less than about 1, 2, 3, 4, 5, 6, 7, 8, 9, 10, 15, 20, 25, 30, 35, 40, 45, 50, 60, 65, 70, 75, 80, 85, 90, 95, 99 or about 100% of the total recombinant β-lactoglobulin proteins in the composition, and various ranges may be selected from between any of these values, for example, from about 1 to about 99, about 1 to about 90, about 1 to about 80, about 1 to about 75, about 1 to about 70, about 1 to about 65, about 1 to about 60, about 1 to about 55, about 1 to about 50, about 1 to about 45, about 1 to about 40, about 1 to about 35, about 1 to about 30, about 1 to about 25, about 1 to about 20, about 1 to about 15, about 1 to about 10, or about 1 to about 5% of the total recombinant β-lactoglobulin proteins in the composition.

In various embodiments the food product or composition may comprise a recombinant, elongated β-lactoglobulin protein.

In various embodiments the food product or composition may comprise one or more, two or more, three or more or four or more recombinant elongated β-lactoglobulin proteins of differing amino acid sequence. In various embodiments the food product or composition may comprise at least 1, 2, 3, 4, 5, 6, 7, 8, 9, 10, 12, 14, 15, 16, 18 or 20 recombinant elongated β-lactoglobulin proteins of differing amino acid sequence, and suitable ranges may be selected from between any of these values, for example, from about 1 to about 20, about 1 to about 15, about 1 to about 10, about 1 to about 5, about 2 to about 20, about 2 to about 15, about 2 to about 10, about 2 to about 5, about 3 to about 20, about 3 to about 15, about 3 to about 10, or about 3 to about 5.

In various embodiments the one or more recombinant, elongated β-lactoglobulin proteins may have an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of from about 1 to about 20 amino acids.

In various embodiments the one or more recombinant, elongated β-lactoglobulin proteins may have an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of 1 amino acid or 2 or more contiguous amino acids from the sequence KREAEAM, KREAEAEAM, or KREAEAEAEAM.

In various embodiments the recombinant, elongated β-lactoglobulin protein may have an amino acid sequence comprising or consisting of a core sequence having at least about 70, 75, 80, 85, 90, 95, 99 or 100% sequence identity to
a) a wild type, mature β-lactoglobulin;
b) any one of SEQ ID Nos:1 to 3, 34, 36, 38, 40, 42, 44, 45, or 47; c) any one of SEQ ID Nos:1-3;
d) SEQ ID No:1 or 2;
e) SEQ ID No:1; or
f) SEQ ID No:2.

In various embodiments the N-terminal elongation may have a sequence comprising or consisting of 1 amino acid or 2 or more contiguous amino acids from the sequence

| | |
|---|---|
| a) | REAEAEAEAM, REAEAEAM, or REAEAM |
| b) | EAEAEAEAM, EAEAEAM, or EAEAM; |
| | or |
| c) | EAEAEAEA, EAEAEA, or EAEA. |

In various embodiments the one or more recombinant, elongated β-lactoglobulin proteins may have an N-terminal elongation having a sequence comprising or consisting of any one of:
a) EAEAM
b) EAEA
c) EAM
d) EA
e) three or more repeats of EA
f) M; and
g) R.

In various embodiments the composition further comprises
a) one or more recombinant, truncated β-lactoglobulin proteins that comprise or consist of an amino acid sequence having an N-terminal truncation of from about 1 to about 20 amino acids relative to the sequence of a wild type, mature β-lactoglobulin; and/or
b) one or more recombinant, substituted β-lactoglobulin proteins that comprise or consist of an amino acid sequence having one or more substitutions of from about 1 to about 20 amino acids relative to the sequence of a wild type, mature β-lactoglobulin.

In various embodiments the amount of modified β-lactoglobulin protein comprising or consisting of an amino acid sequence that comprises an N-terminal elongation of from about 1 to about 20 amino acids relative to the sequence of any one of a) a wild type, mature β-lactoglobulin; b) SEQ ID Nos:1 to 3, 34, 36, 38, 40, 42, 44, 45, or 47; c) SEQ ID Nos 1 to 3, or d) SEQ ID No 1 or 2, is at least about 1, 5, 10, 15, 20, 25, 30, 35, 40, 45, 50, 60, 65, 70, 75, 80, 85, 90, 95, 99 or about 100% of the total recombinant β-lactoglobulin proteins in the composition, and various ranges may be selected from between any of these values, for example, from about 0 to about 100, about 0 to about 99, about 0 to about 90, about 0 to about 80, about 0 to about 70, about 0 to about 60, about 0 to about 50, about 0 to about 40, about 0 to about 30, about 0 to about 20, about 0 to about 10, about 1 to about 100, about 1 to about 99, about 1 to about 90, about 1 to about 80, about 1 to about 70, about 1 to about 60, about 1 to about 50, about 10 to about 100, about 10 to about 99, about 10 to about 90, about 10 to about 80, about 10 to about 70, about 10 to about 60, about 10 to about 50, about 20 to about 100, about 20 to about 99, about 20 to about 90, about 20 to about 80, about 20 to about 70, about 20 to about 60, about 20 to about 50, about 30 to about 100, about 30 to about 99, about 30 to about 90, about 30 to about 80, about 30 to about 70, about 30 to about 60, about 30 to about 50, about 40 to about 100, about 40 to about 99, about 40 to about 90, about 40 to about 80, about 40 to about 70, about 40 to about 60, about 40 to about 50, about 50 to about 100, about 50 to about 99, about 50 to about 90, about 50 to about 80, about 50 to about 70, about 50 to about 60, about 60 to about 100, about 60 to about 99, about 60 to about 90, about 60 to about 80, about 60 to about 70, about 70 to about 100, about 70 to about 99, about 70 to about 90, about 70 to about 80, about 80 to about 100, about 80 to about 99, or about 80 to about 90% of the total recombinant β-lactoglobulin proteins in the composition.

In various embodiments the amount of the one or more recombinant, elongated β-lactoglobulin proteins in the composition may be at least about 1, 2, 3, 4, 5, 10, 15, 20, 25, 30, 35, 40, 45, 50, 60, 65, 70, 75, 80, 85, 90, 95, 99 or about 100% of the total recombinant β-lactoglobulin proteins in the composition, and various ranges may be selected from between any of these values, for example, from about 0 to about 100, about 0 to about 99, about 0 to about 90, about 0 to about 80, about 0 to about 70, about 0 to about 60, about 0 to about 50, about 0 to about 40, about 0 to about 30, about 0 to about 20, about 0 to about 10, about 1 to about 100, about 1 to about 99, about 1 to about 90, about 1 to about 80, about 1 to about 70, about 1 to about 60, about 1 to about 50, about 10 to about 100, about 10 to about 99, about 10 to about 90, about 10 to about 80, about 10 to about 70, about 10 to about 60, about 10 to about 50, about 20 to about 100, about 20 to about 99, about 20 to about 90, about 20 to about 80, about 20 to about 70, about 20 to about 60, about 20 to about 50, about 30 to about 100, about 30 to about 99, about 30 to about 90, about 30 to about 80, about 30 to about 70, about 30 to about 60, about 30 to about 50, about 40 to about 100, about 40 to about 99, about 40 to about 90, about 40 to about 80, about 40 to about 70, about 40 to about 60, about 40 to about 50, about 50 to about 100, about 50 to about 99, about 50 to about 90, about 50 to about 80, about 50 to about 70, about 50 to about 60, about 60 to about 100, about 60 to about 99, about 60 to about 90, about 60 to about 80, about 60 to about 70, about 70 to about 100, about 70 to about 99, about 70 to about 90, about 70 to about 80, about 80 to about 100, about 80 to about 99, or about 80 to about 90% of the total recombinant β-lactoglobulin proteins in the composition.

In various embodiments the amount of modified β-lactoglobulin protein comprising or consisting of an amino acid sequence that comprises an N-terminal truncation of from about 1 to about 20 amino acids relative to the sequence of any one of a) a wild type, mature β-lactoglobulin; b) SEQ ID Nos:1 to 3, 34, 36, 38, 40, 42, 44, 45, or 47; c) SEQ ID Nos 1 to 3, or d) SEQ ID No 1 or 2, is at least about 1, 5, 10, 15, 20, 25, 30, 35, 40, 45, 50, 60, 65, 70, 75, 80, 85, 90, 95, 99 or about 100% of the total recombinant β-lactoglobulin proteins in the composition, and various ranges may be selected from between any of these values, for example, from about 1 to about 100, about 1 to about 99, about 1 to about 90, about 1 to about 80, about 1 to about 70, about 1 to about 60, about 1 to about 50, about 10 to about 100, about 10 to about 99, about 10 to about 90, about 10 to about 80, about 10 to about 70, about 10 to about 60, about 10 to about 50, about 20 to about 100, about 20 to about 99, about 20 to about 90, about 20 to about 80, about 20 to about 70, about 20 to about 60, about 20 to about 50, about 30 to about 100, about 30 to about 99, about 30 to about 90, about 30 to about 80, about 30 to about 70, about 30 to about 60, about 30 to about 50, about 40 to about 100, about 40 to about 99, about 40 to about 90, about 40 to about 80, about 40 to about 70, about 40 to about 60, about 40 to about 50, about 50 to about 100, about 50 to about 99, about 50 to about 90, about 50 to about 80, about 50 to about 70, about 50 to about 60, about 60 to about 100, about 60 to about 99, about 60 to about 90, about 60 to about 80, about 60 to about 70, about 70 to about 100, about 70 to about 99, about 70 to about 90, about 70 to about 80, about 80 to about 100, about 80 to about 99, or about 80 to about 90 of the total recombinant β-lactoglobulin proteins in the composition.

In various embodiments:
a) the amount of recombinant β-lactoglobulin protein comprising or consisting of an amino acid sequence of any one of SEQ ID Nos 1 to 3, is from about 1 to about 10% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of any one of SEQ ID Nos 1 to 3, is about 50 to about 99% of the total recombinant β-lactoglobulin proteins in the composition; and
c) the amount of modified β-lactoglobulin protein comprising or consisting of an amino acid sequence that comprises an N-terminal truncation of from about 1 to about 20 amino acids relative to the sequence of any one of SEQ ID Nos 1 to 3, is at least about 30% of the total recombinant β-lactoglobulin proteins in the composition.

In various embodiments:
a) the amount of recombinant β-lactoglobulin protein comprising or consisting of an amino acid sequence of any one of SEQ ID Nos 1 to 3, is from about 1 to about 50% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of any one of SEQ ID Nos 1 to 3, is about 0 to about 10% of the total recombinant β-lactoglobulin proteins in the composition; and
c) the amount of modified β-lactoglobulin protein comprising or consisting of an amino acid sequence that comprises an N-terminal truncation of from about 1 to about 20 amino acids relative to the sequence of any one of SEQ ID Nos 1 to 3, is from about 50 to about 99% of the total recombinant β-lactoglobulin proteins in the composition.

In various embodiments the modified β-lactoglobulin protein comprises one or more recombinant proteins each comprising or consisting of a sequence of any one of SEQ ID Nos: 4-7 and SEQ ID Nos: 19-22. In various embodiments the modified β-lactoglobulin protein comprises one or more recombinant proteins each comprising or consisting of a sequence of any one of SEQ ID Nos: 4-7, 19-22 and 71-74.

In various embodiments the composition or food product comprises one or more recombinant, elongated β-lactoglobulin proteins comprising or consisting of a sequence of any one of SEQ ID Nos: 4-7, 19-22 and 71-74.

In various embodiments the composition or food product comprises two or more, three or more, five or more, six or more or seven or more recombinant, elongated β-lactoglobulin proteins of differing amino acid sequence. In various embodiments the composition comprises at least about 1, 2, 3, 4, 5, 6, 7, 8, 9, 10, 12, 14, 15, 16, 18 or 20 recombinant, elongated β-lactoglobulin proteins of differing amino acid sequence, and various ranges may be selected from between these values, for example from about 1 to about 20, about 1 to about 15, about 1 to about 10, about 1 to about 8, about 1 to about 6, about 1 to about 5, or about 2 to about 20, about 2 to about 15, about 2 to about 10, about 2 to about 8, about 2 to about 6, about 2 to about 5, or about 3 to about 20, or about 3 to about 10 recombinant, elongated β-lactoglobulin proteins of differing amino acid sequence. In various embodiments the composition or food product may comprise one or more, two or more, three or more or four or more recombinant β-lactoglobulin proteins having an amino acid sequence selected from SEQ ID Nos: 4-7, 19-22 and 71-74.

In various embodiments the composition or food product may comprise one or more, two or more, three or more or four or more recombinant β-lactoglobulin proteins having an amino acid sequence selected from
a) SEQ ID Nos:4-7, 71 and 72; or
b) SEQ ID Nos:19-22, 73 and 74.

In various embodiments the modified β-lactoglobulin protein comprises one or more recombinant proteins each comprising or consisting of a sequence of any one of SEQ ID Nos: 8-18, SEQ ID Nos: 23-30 and SEQ ID NO: 61.

In various embodiments the plurality of recombinant β-lactoglobulin proteins has a secondary protein structure comprising or consisting of:
a) from about 0 to about 6% α-helix;
b) from about 40 to about 55% anti-parallel β-sheet;
c) from about 0 to about 3% parallel β-sheet;
d) from about 8 to about 20% turns; and
e) about 35 to about 45% unspecified or unordered structure.

In various embodiments, wherein the wild type, mature β-lactoglobulin has a sequence of SEQ ID No:1, the plurality of recombinant β-lactoglobulin proteins has a secondary protein structure comprising or consisting of
a) from about 0.5 to about 6%, about 1 to about 6% or about 2 to about 6% α-helix;
b) from about 40 to about 55%, about 40 to about 50%, or about 40 to about 45% anti-parallel β-sheet;
c) from about 0 to about 3%, or about 0 to about 2.5%, parallel β-sheet;
d) from about 8 to about 20% about 10 to about 20%, or about 10 to about 15% turns; and
e) about 35 to about 45% unspecified or unordered structure.

In various embodiments, wherein the wild type, mature β-lactoglobulin has a sequence of SEQ ID No:2, the plurality of recombinant β-lactoglobulin proteins has a secondary protein structure comprising or consisting of
a) from about 0 to about 5%, about 0 to about 4%, about 0 to about 3% or about 0 to about 2% or about 0 to about 1% α-helix;
b) from about 40 to about 55%, about 45 to about 55%, or about 45 to about 50% anti-parallel β-sheet;
c) from about 0 to about 3%, about 0 to about 2%, or about 0 to about 1%, parallel β-sheet;
d) from about 8 to about 20% about 10 to about 20% or about 10 to about 15% turns; and
e) about 35 to about 45% unspecified or unordered structure.

In various embodiments the plurality of recombinant β-lactoglobulin proteins has a mean DIAAS score of at least 0.8.

In various embodiments the plurality of recombinant β-lactoglobulin proteins denatures at a higher temperature than a protein consisting of the sequence of SEQ ID No:1 or 2 as measured by differential scanning calorimetry.

In various embodiments, the plurality of recombinant β-lactoglobulin proteins denatures at a lower temperature than a protein consisting of the sequence of SEQ ID No:1 or 2 as measured by differential scanning calorimetry.

In various embodiments the storage modulus of a heat set gel comprising the composition at a pH of about 7 is at least equivalent to that of NZMP SureProtein™ WPI895 when measured at a frequency of 1 Hz and at 20° C., wherein the heat set gel is formed by heating a solution comprising 10% by weight of the plurality of recombinant β-lactoglobulin proteins or WPI895 by heating from 20 to 80° C. at a rate of 1° C./min, holding the solution at 80° C. for 30 min, and cooling the solution from 80 to 20° C. at a rate of 1° C./min and holding the temperature at 20° C. for 20 min on a rheometer to form the gel.

In various embodiments the food product is a yoghurt and the yoghurt exhibits increased, similar or reduced firmness and/or viscosity compared with a control food product having the same ingredient composition and total protein content, except that the recombinant β-lactoglobulin protein is replaced with milk-derived β-lactoglobulin.

In various embodiments the stiffness (G′) at 20° C. of a heat set gel comprising the composition at a pH of about 7 is at least about 7,000 when measured at a frequency of 0.1 Hz and a strain of 0.25%, wherein the heat set gel is formed by heating a solution comprising 10% by weight of the protein(s) from 20° C. to 90° C. at a rate of 5° C./min, holding at 90° C. for 30 min, and cooling to 20° C. at a rate of 5° C./min. In various embodiments, the stiffness (G′) is at most about 30,000, or from 8,000 to 30,000.

In various embodiments the stiffness (G′) at 20° C. of a heat set gel comprising the composition at a pH of about 7 is at least about 80% of that of a heat set gel formed with wild type β-lactoglobulin when measured at a frequency of 0.1 Hz and a strain of 0.25%, wherein the heat set gel is formed by heating a solution comprising 10% by weight of the protein(s) from 20° C. to 90° C. at a rate of 5° C./min, holding at 90° C. for 30 min, and cooling to 20° C. at a rate of 5° C./min. In various embodiments, the stiffness (G′) is at most about 200%, or from 85% to 180% of that of a heat set gel formed with wildtype β-lactoglobulin.

In various embodiments the final stiffness of an acid-set gel formed by combining milk and 1.0% by weight of the protein(s), heating to 80° C. for 30 minutes, cooling to 30° C., and acidifying with 2% w/w glucono-δ-lactone (GDL) is at least 400 Pa, or at least 100% of that of an acid-set gel formed using wild type bovine β-lactoglobulin.

In various embodiments a shelf-stable protein beverage comprising 3.5% by weight of the plurality of recombinant β-lactoglobulin proteins exhibits no visible sedimentation after 4 weeks at ambient temperature.

In various embodiments a solution having a pH of about 7 and comprising 10% by weight of the plurality of recombinant β-lactoglobulin proteins has an overrun of at least 500% after 10 minutes whipping, and/or wherein a foam produced by whipping a solution of neutral pH comprising 10% by weight of the plurality of recombinant proteins exhibits no serum leakage for at least about 5 minutes after whipping.

In various embodiments a solution of neutral pH comprising 2.5% by weight of the plurality of recombinant proteins has a foam volume after 70 seconds whipping of at least 75 mL, and/or produces a foam after 70 seconds of whipping that retains at least 50% of its volume after 45 minutes.

In various embodiments an emulsion comprising the composition exhibits increased, the same or reduced stability compared with an emulsion comprising a protein having a sequence of any one of SEQ ID Nos:1 or 2, wherein the emulsion is formed by combining the composition with oil to in an amount of from 2.5 to 17.5% and water, pre-homogenizing and homogenizing by sonication at 100% amplitude for 10 min, and measuring particle size using laser light scattering.

In various embodiments the emulsion activity index (EAI) of an emulsion comprising the composition is increased, the same or decreased compared with an emulsion comprising a protein having a sequence of SEQ ID Nos:1 or 2, wherein the emulsion is formed by preparing a solution comprising 0.5% of the plurality of recombinant β-lactoglobulin proteins, 20% soya oil and water and homogenizing the solution at 200 bar, and wherein the EAI is determined by measuring the specific surface area of the emulsion droplets using laser light scattering.

In various embodiments, an emulsion comprising the composition exhibits increased, the same or decreased emulsifying capacity (EC) compared with an emulsion comprising a protein of SEQ ID No:1 or 2. In some embodiments the emulsion is formed by combining the composition, using different levels of oil to give a range of oil to protein ratios and homogenizing and immediately measuring the electrical conductivity to determine EC.

In various embodiments, an emulsion comprising a composition with 0.5% w/w protein has an EC of at least 900 mL oil/g protein and/or has an EC of at least 90% of that of wild type β-lactoglobulin.

In various embodiments the composition may be substantially free of aspartyl protease-like activity.

In various embodiments, when the composition is added to a skim milk composition comprising 10% by weight total solids to a concentration of the one or more recombinant proteins of about 1% by weight and the skim milk composition is heated at 80° C. for 30 minutes then held at 5° C. for 6 hours,
a) no degradation of κ-casein is observed, and/or
b) no production of para-κ-casein is observed.

In various embodiments, when the composition is added to a skim milk composition comprising 10% by weight total solids to a concentration of the one or more recombinant proteins of about 1% by weight and incubated at ambient temperature for 24 hours, less than about 45%, 40%, 35%, 30%, 25%, 20%, 15%, 10%, 5% or 1%, or 0% of κ-casein in the skim milk composition is degraded to form para-κ-casein.

In various embodiments, when the composition is added to a skim milk composition comprising 10% by weight total solids to a concentration of the one or more recombinant proteins of about 1% by weight and the skim milk composition is held at ambient temperature for 15 minutes then heated at 140° C., the composition has not coagulated after about 14 minutes.

In some embodiments, when the composition is added to a skim milk composition comprising 10% by weight total solids to a concentration of the one or more recombinant proteins of about 1% by weight, and the skim milk composition is heated at 40° C. for 12 hours, the skim milk composition does not reach gelation point.

In various embodiments the composition is substantially free of aspartyl protease
a) consisting of an amino acid sequence of SEQ ID no. 62 or 63, or
b) having at least 75%, 80%, 85%, 90%, 95% or 99% sequence identity to SEQ ID Nos: 62 or 63.

In various embodiments the food product is in bar or solid moulded form. “Solid moulded form” means that the food product has been moulded into a shape that holds its form.

In various embodiments the food product may be selected from the group consisting of a fermented food, a yoghurt, a soup, a sauce, a bar, a gel, a foam, a nutritional formulation, a beverage, a beverage whitener, a cheese, a dairy tofu, a food emulsion and a dessert.

In various embodiments the food product may be a yoghurt, drinking yoghurt, a bar, a gel, a foam, a nutritional formulation, medical food, dairy beverage, a product that requires the protein to form a heat-set gel, an acid protein fortified beverage, a jelly drink, a protein water, a heat-set foam extruded food product, or a food emulsion.

In various embodiments the nutritional formulation or food may be an infant formula, toddler milk, growing up formula, maternal formula, a sports beverage, food for active lifestyles, a medical food or supplement.

In various embodiments the nutritional formulation may be selected from the group comprising an infant formula, a follow-on formula, a toddler milk, a growing up formula, a maternal formula, a food for active lifestyles, a medical food and a supplement.

In various embodiments the cheese may be fresh cheese. In various embodiments the cheese may be processed cheese, Petit-Suisse, cottage cheese, or quark.

In various embodiments the beverage is selected from the group comprising a dairy beverage, a sports beverage, a smoothie, a protein fortified fruit or vegetable juice, a drinking yoghurt, an acid protein fortified beverage, a jelly drink, protein water, liquid coffee, liquid tea, and a liquid beverage whitener.

In various embodiments the food product may comprise an additional source of protein. In various embodiments the additional source of protein is a non-dairy source. In various embodiments the non-dairy source may comprise a plant source, algal protein, mycoprotein, or a combination thereof.

In various embodiments the plant source may comprise one or more legumes, grains, seeds, nuts, tubers, or any combination of any two or more thereof.

In various embodiments
a) the legumes may comprise soy, pea, lentil, chickpea, peanut, bean or any combination of any two or more thereof;
b) the grains may comprise wheat, rice, oat, corn or any combination of any two or more thereof;
c) the seeds may comprise canola, flaxseed (linseed), hemp, sunflower, quinoa, chia, or any combination of any two or more thereof;
d) the nuts may comprise almond, cashew, walnut or any combination of any two or more thereof; and/or
e) the tuber may comprise potato.

In various embodiments the food product is suitable for those on a vegan diet.

In various embodiments the food product may not comprise any milk proteins other than the recombinant, β-lactoglobulin proteins.

In various embodiments the food product comprises casein. In various embodiments the food product comprises κ-casein.

In various embodiments the method for preparing the food product comprises mixing the composition with one or more additional ingredients to produce the food product, preferably wherein at least one ingredient comprises κ-casein.

In various embodiments the food product comprises milk-derived casein, one or more recombinant casein proteins, or a combination thereof.

In various embodiments the food product comprises milk-derived κ-casein, recombinant κ-casein, or a combination thereof.

In various embodiments the food product or the one or more additional ingredients may comprise whole milk, skim milk, a milk protein concentrate (MPC), a milk protein isolate (MPI), micellar casein, or a combination of any two or more thereof.

The term “heterogeneous” as used herein with reference to a plurality of recombinant proteins means that the plurality of recombinant proteins comprises at least two or two or more, three or more, four or more, five or more, six or more, or seven or more proteins of differing amino acid sequence.

The term “mature” as used herein with reference to β-lactoglobulin proteins refers to the β-lactoglobulin protein, or amino acid sequence of the protein, after cleavage of the signal sequence. The term “full length” as used herein with reference to β-lactoglobulin proteins refers to the β-lactoglobulin protein, or amino acid sequence of the protein, comprising the signal sequence. Examples of mature and full length β-lactoglobulin sequences are provided in Table 1 herein.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The terms “variant” or “modified β-lactoglobulin protein” as used herein may include proteins comprising an amino acid sequence having at least about 70%, 75%, 80%, 85%, 90%, 95%, 96%, 97%, 98% or at least about 99% sequence identity to the sequence of a wild type (native) β-lactoglobulin (either full length or mature β-lactoglobulin lacking a signal sequence, but preferably the mature sequence), but particularly any wild type bovine, ovine, caprine, buffalo, equine, donkey or reindeer β-lactoglobulin sequence, including any sequence of SEQ ID Nos:1 to 3 or 31-48. In some embodiments the amino acid sequence of such variants may comprise truncations or elongations at the N-terminus and/or the C-terminus relative to the wild type sequence, for example, elongations or truncations of from about 1 to about 20 amino acids. In some embodiments, variants or modified β-lactoglobulin proteins may contain from 1 to 20 amino acid insertions, deletions, and/or substitutions (collectively) with respect to the wild type sequence. Such proteins may be referred to herein as “elongated β-lactoglobulin proteins”, “truncated β-lactoglobulin proteins” or “substituted β-lactoglobulin proteins”. In some embodiments the variants or modified β-lactoglobulin proteins may comprise one or more post-translational modifications that differ to a wild type β-lactoglobulin protein, including glycosylation and or phosphorylation at one or more residues.

The term “wild type” as used herein with reference to proteins or polynucleotides refers to a protein or polynucleotide having an amino acid or nucleotide sequences that is the same as that expressed naturally in any species. This term includes all naturally occurring variants of a particular protein, for example, all naturally occurring variants of β-lactoglobulin. Furthermore, this term includes both full length β-lactoglobulin proteins and mature β-lactoglobulin proteins and polynucleotides that encode wild type full length and mature β-lactoglobulin. The term is generally synonymous with the term “native”.

The term “comprising” as used in this specification and claims means “consisting at least in part of”. When interpreting statements in this specification and claims which include the term “comprising”, other features besides the features prefaced by this term in each statement can also be present. Related terms such as “comprise” and “comprised” are to be interpreted in similar manner.

As used herein the term “and/or” means “and” or “or”, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The invention will now be described by way of example only and with reference to the drawings in which:

FIG. 1 provides plasmid maps of the following expression constructs: (A) pLGAAOX-005 for expression of β-lactoglobulin variant A; and (B) pLGBAOX-005 for expression of the β-lactoglobulin variant B.

FIG. 2 provides a plasmid map of the expression construct pLGATOP-002 for expression of the β-lactoglobulin variant A.

FIG. 3 provides a plasmid map of the expression construct pLGBTOP-002 for expression of the β-lactoglobulin variant B.

FIG. 4 provides a map of the marker-gene containing vector pGBAAS-3.

FIG. 5 provides a plasmid map of the expression construct pCASPP-05.

### DETAILED DESCRIPTION OF THE INVENTION

The present disclosure generally relates to protein compositions comprising a plurality of recombinant β-lactoglobulin proteins heterogeneous in amino acid sequence, wherein the plurality comprises at least one modified β-lactoglobulin protein comprising an amino acid sequence comprising an N-terminal truncation, an N-terminal elongation and/or one or more substitutions relative to the sequence of a wild type, mature β-lactoglobulin. More specifically, the invention relates to elongated β-lactoglobulin proteins and compositions comprising two or more recombinant β-lactoglobulin proteins of differing amino acid sequence, including at least one recombinant, elongated β-lactoglobulin protein.

The invention also relates to methods of producing the compositions and the use of the compositions as functional ingredients in the preparation of food products including yoghurts, drinking yoghurts, bars, gels, foams, nutritional formulations, medical foods, dairy beverages, products that comprise protein in the form of a heat-set gel, acid protein fortified beverages, jelly drinks, protein water, heat-set foam extruded food products, and food emulsions.

The proteins and compositions confer certain advantages when used as functional ingredients in foods including solubility over a wide pH range, the ability to form desirable heat-set gels with water holding properties, surface active properties such as foaming and emulsification, heat stability at low protein addition rates, desirable heat-induced interactions with casein proteins to modify functional properties in acid gel applications, improved nutrition, structure and cohesiveness when used in bars.

The proteins and compositions may also provide for the preparation of food products having similar characteristics to dairy food products.

### 1. Proteins and Protein Compositions

β-lactoglobulin is the major whey protein in the milk of many mammals. In bovine milk it accounts for approximately 10-15% of total milk proteins and about 50-54% of whey protein.

Bovine β-lactoglobulin is expressed as a precursor protein comprising a 16 amino acid N-terminal signal peptide (referred to herein and elsewhere as the “full-length” β-lactoglobulin protein), which is cleaved to form a mature 162 amino acid protein.

There are two primary variants of bovine β-lactoglobulin—variants A and B and a lesser variant—variant C. Sequences for both the mature and full length forms of bovine β-lactoglobulin variants A, B and C, and wild type full length and mature forms of β-lactoglobulin from other species are presented in Table 1.

TABLE 1
Sequences of wild type β-lactoglobulin with signal sequence bolded.

| SEQ ID | | |
|---|---|---|
| No. | Name | Sequence |
| 1 | Bovine β- | LIVTQTMKGLDIQKVAGTWYSLAMAASDISLLDAQSAPLR |
| | lactoglobulin | VYVEELKPTPEGDLEILLQKWENDECAQKKIIAEKTKIPAV |
| | variant A | FKIDALNENKVLVLDTDYKKYLLFCMENSAEPEQSLVCQCL |
| | (mature) | VRTPEVDDEALEKFDKALKALPMHIRLSFNPTQLEEQCHI |
| 31 | Bovine β- | MKCLLLALALTCGAQALIVTQTMKGLDIQKVAGTWYSL |
| | lactoglobulin | AMAASDISLLDAQSAPLRVYVEELKPTPEGDLEILLQKWE |
| | variant A (full | NDECAQKKIIAEKTKIPAVFKIDALNENKVLVLDTDYKKYL |
| | length) | LFCMENSAEPEQSLVCQCLVRTPEVDDEALEKFDKALKAL |
| | | PMHIRLSFNPTQLEEQCHI |
| 2 | Bovine β- | LIVTQTMKGLDIQKVAGTWYSLAMAASDISLLDAQSAPLR |
| | lactoglobulin | VYVEELKPTPEGDLEILLQKWENGECAQKKIIAEKTKIPAV |
| | variant B | FKIDALNENKVLVLDTDYKKYLLFCMENSAEPEQSLACQCL |
| | (mature) | VRTPEVDDEALEKFDKALKALPMHIRLSFNPTQLEEQCHI |
| 32 | Bovine β- | MKCLLLALALTCGAQALIVTQTMKGLDIQKVAGTWYSL |
| | lactoglobulin | AMAASDISLLDAQSAPLRVYVEELKPTPEGDLEILLQKWE |
| | variant B (full | NGECAQKKIIAEKTKIPAVFKIDALNENKVLVLDTDYKKYL |
| | length) | LFCMENSAEPEQSLACQCLVRTPEVDDEALEKFDKALKAL |
| | | PMHIRLSFNPTQLEEQCHI |
| 3 | Bovine β- | LIVTQTMKGLDIQKVAGTWYSLAMAASDISLLDAQSAPLR |
| | lactoglobulin | VYVEELKPTPEGDLEILLHKWENGECAQKKIIAEKTKIPAV |
| | variant C | FKIDALNENKVLVLDTDYKKYLLFCMENSAEPEQSLACQCL |
| | (mature) | VRTPEVDDEALEKFDKALKALPMHIRLSFNPTQLEEQCHI |
| 33 | Bovine β- | MKCLLLALALTCGAQALIVTQTMKGLDIQKVAGTWYSL |
| | lactoglobulin | AMAASDISLLDAQSAPLRVYVEELKPTPEGDLEILLHKWEN |
| | variant C (full | GECAQKKIIAEKTKIPAVFKIDALNENKVLVLDTDYKKYLLF |
| | length) | CMENSAEPEQSLACQCLVRTPEVDDEALEKFDKALKALPM |
| | | HIRLSFNPTQLEEQCHI |
| 34 | Ovine β- | IIVTQTMKGLDIQKVAGTWHSLAMAASDISLLDAQSAPLR |
| | lactoglobulin | VYVEELKPTPEGNLEILLQKWENGECAQKKIIAEKTKIPAV |
| | (mature) | FKIDALNENKVLVLDTDYKKYLLFCMENSAEPEQSLACQCL |
| | | VRTPEVDNEALEKFDKALKALPMHIRLAFNPTQLEGQCHV |
| 35 | Ovine β- | MKCLLLALGLALACGVQAIIVTQTMKGLDIQKVAGTWH |
| | lactoglobulin (full | SLAMAASDISLLDAQSAPLRVYVEELKPTPEGNLEILLQKW |
| | length) | ENGECAQKKIIAEKTKIPAVFKIDALNENKVLVLDTDYKKY |
| | | LLFCMENSAEPEQSLACQCLVRTPEVDNEALEKFDKALKA |
| | | LPMHIRLAFNPTQLEGQCHV |
| 36 | Caprine β- | IIVTQTMKGLDIQKVAGTWYSLAMAASDISLLDAQSAPLR |
| | lactoglobulin | VYVEELKPTPEGNLEILLQKWENGECAQKKIIAEKTKIPAV |
| | (mature) | FKIDALNENKVLVLDTDYKKYLLFCMENSAEPEQSLACQCL |
| | | VRTPEVDKEALEKFDKALKALPMHIRLAFNPTQLEGQCHV |
| 37 | Caprine β- | MKCLLLALGLALACGIQAIIVTQTMKGLDIQKVAGTWY |
| | lactoglobulin (full | SLAMAASDISLLDAQSAPLRVYVEELKPTPEGNLEILLQKW |
| | length) | ENGECAQKKIIAEKTKIPAVFKIDALNENKVLVLDTDYKKY |
| | | LLFCMENSAEPEQSLACQCLVRTPEVDKEALEKFDKALKAL |
| | | PMHIRLAFNPTQLEGQCHV |
| 38 | Water buffalo β- | IIVTQTMKGLDIQKVAGTWYSLAMAASDISLLDAQSAPLR |
| | lactoglobulin | VYVEELKPTPEGDLEILLQKWENGECAQKKIIAEKTKIPAV |
| | (mature) | FKIDALNENKVLVLDTDYKKYLLFCMENSAEPEQSLACQCL |
| | | VRTPEVDDEALEKFDKALKALPMHIRLSFNPTQLEEQCHV |
| 39 | Water buffalo β- | MKCLLLALGLALACAAQAIIVTQTMKGLDIQKVAGTWY |
| | lactoglobulin (full | SLAMAASDISLLDAQSAPLRVYVEELKPTPEGDLEILLQKW |
| | length) | ENGECAQKKIIAEKTKIPAVFKIDALNENKVLVLDTDYKKY |
| | | LLFCMENSAEPEQSLACQCLVRTPEVDDEALEKFDKALKA |
| | | LPMHIRLSFNPTQLEEQCHV |
| 40 | Equine β- | TNIPQTMQDLDLQEVAGKWHSVAMAASDISLLDSESAPL |
| | lactoglobulin | RVYIEKLRPTPEDNLEIILREGENKGCAEKKIFAEKTESPAE |
| | variant I | FKINYLDEDTVFALDTDYKNYLFLCMKNAATPGQSLVCQY |
| | (mature) | LARTQMVDEEIMEKFRRALQPLPGRVQIVPDLTRMAERCR |
| | | I |
| 41 | Equine β- | MKCLLLALGLALMCGIQATNIPQTMQDLDLQEVAGKW |
| | lactoglobulin | HSVAMAASDISLLDSESAPLRVYIEKLRPTPEDNLEIILREG |
| | variant I (full | ENKGCAEKKIFAEKTESPAEFKINYLDEDTVFALDTDYKNY |
| | length) | LFLCMKNAATPGQSLVCQYLARTQMVDEEIMEKFRRALQP |
| | | LPGRVQIVPDLTRMAERCRI |
| 42 | Equine β- | TDIPQTMQDLDLQEVAGRWHSVAMVASDISLLDSESVPL |
| | lactoglobulin | RVYVEELRPTPEGNLEIILREGANHACVERNIVAQKTEDPA |
| | variant II | VFTVNYQGERKISVLDTDYAHYMFFCVGPPLPSAEHGMVC |
| | (mature) | QYLARTQKVDEEVMEKFSRALQPLPGRVQIVQDPSGGQE |
| | | RCGF |
| 43 | Equine β- | MKCLLLALGLSLMCGNQATDIPQTMQDLDLQEVAGRW |
| | lactoglobulin | HSVAMVASDISLLDSESVPLRVYVEELRPTPEGNLEIILRE |
| | variant II (full | GANHACVERNIVAQKTEDPAVFTVNYQGERKISVLDTDYA |
| | length) | HYMFFCVGPPLPSAEHGMVCQYLARTQKVDEEVMEKFSR |
| | | ALQPLPGRVQIVQDPSGGQERCGF |
| 44 | Donkey β- | TNIPQTMQDLDLQEVAGKWHSVAMAASDISLLDSEEAPL |
| | lactoglobulin | RVYIEKLRPTPEDNLEIILREGENKGCAEKKIFAEKTESPAE |
| | variant 1 | FKINYLDEDTVFALDSDYKNYLFLCMKNAATPGQSLVCQY |
| | (mature) | LARTQMVDEEIMEKFRRALQPLPGRVQIVPDLTRMAERCR |
| | | I |
| 45 | Donkey β- | TDIPQTMQDLDLQEVAGRWHSVAMVASDISLLDSESVPL |
| | lactoglobulin | RVYVEELRPTPEGNLEIILREGANHACVERNIVAQKTEDPA |
| | variant 2 | VFTVNYQGERKISVLDTDYAHYMFFCVGPPLPSAEHGMVC |
| | (mature) | QYLARTQKVDEEVMEKFSRALQPLPGRVQIVQDPSGGQE |
| | | RCGF |
| 46 | Donkey | MKCLLLALGLSLMCGNQATDIPQTMQDLDLQEVAGRW |
| | β-lactoglobulin II | HSVAMVASDISLLDSESAPLRVYVEELRPTPEGNLEIILRE |
| | variant B (full | GANHVCVERNIVAQKTEDPAVFTVNYQGERKISVLDTDYA |
| | length) | HYMFFCVGPPLPSAEHGTVCQYLARTQKVDEEVMEKFSRA |
| | | LQPLPGHVQIIQDPSGGQERCGF |
| 47 | Reindeer β- | IIVTQTMKDLDVQKVAGTWYSLAMAASDISLLDAQSAPLR |
| | lactoglobulin | VYVEELKPTPGGDLEILLQKWENGKCAQKKIIAEKTEIPAV |
| | (mature) | FKIDALNENKVLVLDTDYKKYLLFCMENSAEPEQSLACQCL |
| | | VRTPEVDDEAMEKFDKALKALPMHIRLSFNPTQLEEQCRV |
| 48 | Reindeer β- | MKCLLITLGLALACGAQAIIVTQTMKDLDVQKVAGTWY |
| | lactoglobulin (full | SLAMAASDISLLDAQSAPLRVYVEELKPTPGGDLEILLQK |
| | length) | WENGKCAQKKIIAEKTEIPAVFKIDALNENKVLVLDTDYKK |
| | | YLLFCMENSAEPEQSLACQCLVRTPEVDDEAMEKFDKALK |
| | | ALPMHIRLSFNPTQLEEQCRV |

In one aspect the invention relates to a recombinant, elongated β-lactoglobulin protein having an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of
   i) 2 or more contiguous amino acids from the sequence KREAEAM; or
   ii) R.

In another aspect the invention relates to a composition comprising one or more recombinant, elongated β-lactoglobulin proteins of the invention.

In another aspect the invention relates to a recombinant, elongated β-lactoglobulin protein having an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of
   i) 2 or more contiguous amino acids from the sequence KREAEAM; or
   ii) R.

In a further aspect the invention relates to a composition comprising a plurality of recombinant β-lactoglobulin proteins heterogeneous in amino acid sequence, the recombinant proteins having at least 70% sequence identity to a wild type, mature β-lactoglobulin, wherein the plurality comprises at least one modified β-lactoglobulin protein comprising or consisting of an amino acid sequence that comprises
a) an N-terminal truncation of from about 1 to about 20 amino acids relative to the sequence of a wild type, mature β-lactoglobulin;
b) an N-terminal elongation of from about 1 to about 20 amino acids relative to the sequence of a wild type, mature β-lactoglobulin; or
c) one or more substitutions of from about 1 to about 20 amino acids relative to the sequence of a wild type, mature β-lactoglobulin,
   and wherein
B) the amount of modified β-lactoglobulin protein is at least 40% of the total recombinant β-lactoglobulin proteins in the composition;
C) the amount of modified β-lactoglobulin protein comprising or consisting of an amino acid sequence that comprises an N-terminal elongation of from about 1 to about 20 amino acids relative to the sequence of a wild type, mature β-lactoglobulin, is at least about 10% of the total recombinant β-lactoglobulin proteins in the composition;
D) the amount of modified β-lactoglobulin protein comprising or consisting of an amino acid sequence that comprises an N-terminal truncation of from about 1 to about 20 amino acids relative to the sequence of a wild type, mature β-lactoglobulin, is at least about 30% of the total recombinant β-lactoglobulin proteins in the composition; or
E) any combination of any two or more of (A) to (C).

In various embodiments the recombinant proteins may have at least about 75%, 80, 85, 90, 95, 99 or 100% sequence identity to a wild type, mature β-lactoglobulin, for example, any of the wild type, mature β-lactoglobulin sequence provided in Table 1 above. In various embodiments the recombinant proteins may have at least about 75%, 80, 85, 90, 95, 99 or 100% sequence identity to a wild type, bovine mature β-lactoglobulin, such as the sequences of SEQ ID Nos:1 to 3.

The plurality comprises at least one modified β-lactoglobulin protein comprising or consisting of an amino acid sequence that comprises an N-terminal truncation or an N-terminal elongation relative to the sequence of a wild type, mature β-lactoglobulin. A modified β-lactoglobulin protein comprising or consisting of an amino acid sequence that comprises an N-terminal truncation refers to the modified protein lacking 1-20 contiguous amino acids from residue 1 of the sequence of the wild type, mature β-lactoglobulin. A modified β-lactoglobulin protein comprising or consisting of an amino acid sequence that comprises an N-terminal elongation refers to the sequence of the modified protein comprising an additional 1-20 contiguous amino acids at the N-terminus relative to the wild type, mature β-lactoglobulin, that is, 1-20 contiguous amino acids preceding residue 1 of the wild type, mature β-lactoglobulin.

In one embodiment
a) the amount of modified β-lactoglobulin protein is at least about 85% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of a wild type, mature β-lactoglobulin, preferably SEQ ID No:1, is at least about 70, 75 or 80% of the total recombinant β-lactoglobulin proteins in the composition, preferably from about 70 to about 90%; and
c) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:1, is at least about 1, 2, 3, 4, or 5% of the total recombinant β-lactoglobulin proteins in the composition, preferably from about 1 to about 15%.

In one embodiment the modified β-lactoglobulin protein or the composition comprises one or more, 2 or more, 3 or more or 4 or more proteins comprising or consisting of a sequence selected from the group comprising SEQ ID Nos: 4, 5, 6, 8, 9, 10 and 11.

In one embodiment
a) the amount of modified β-lactoglobulin protein is at least about 85% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of a wild type, mature β-lactoglobulin, preferably SEQ ID No:2, is at least about 70, 75 or 80% or 85% of the total recombinant β-lactoglobulin proteins in the composition, preferably from about 80 to about 95%; and
c) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:2, is at least about 1, 2, 3, 4, or 5% of the total recombinant β-lactoglobulin proteins in the composition, preferably from about 1 to about 10%.

In one embodiment the modified β-lactoglobulin protein or the composition comprises one or more, 2 or more, 3 or more or 4 or more proteins comprising or consisting of a sequence selected from the group comprising SEQ ID Nos: 19-20, 23 and 24.

In one embodiment
a) the amount of modified β-lactoglobulin protein is at least about 90 or 95% of the total recombinant β-lactoglobulin proteins in the composition; b) the amount of modified β-lactoglobulin protein comprising or consisting of an amino acid sequence that comprises an N-terminal elongation of from about 1 to about 20 amino acids relative to the sequence of a wild type, mature β-lactoglobulin, preferably SEQ ID No:1, is from about 0 to about 20% of the total recombinant β-lactoglobulin proteins in the composition, preferably from about 0 to about 10%; and
c) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:1, is at least about 80, 85 or 90% of the total recombinant β-lactoglobulin proteins in the composition, preferably from about 80 to about 100%.

In one embodiment the modified β-lactoglobulin protein or the composition comprises one or more, 2 or more, 3 or more or 4 or more proteins comprising or consisting of a sequence selected from the group comprising SEQ ID Nos: 8, 11-15, 17 and 18.

In one embodiment
a) the amount of modified β-lactoglobulin protein is at least about 90% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of a wild type, mature β-lactoglobulin, preferably SEQ ID No:2, is from about 0 to about 20% of the total recombinant β-lactoglobulin proteins in the composition, preferably from about 0 to about 10%; and
c) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:2, from about 0 to about 20% of the total recombinant β-lactoglobulin proteins in the composition, preferably from about 0 to about 10%, is at least about 80, 85 or 90% of the total recombinant β-lactoglobulin proteins in the composition, preferably from about 80 to about 100%.

In one embodiment the modified β-lactoglobulin protein or the composition comprises one or more, 2 or more, 3 or more or 4 or more proteins comprising or consisting of a sequence selected from the group comprising SEQ ID Nos: 23-29.

In one embodiment
a) the amount of modified β-lactoglobulin protein is at least about 70 or 80% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of a wild type, mature β-lactoglobulin, preferably SEQ ID No:1, is at least about 1, 2 or 3% of the total recombinant β-lactoglobulin proteins in the composition, preferably from about 1 to about 8%; and
c) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:1, is at least about 50, 55, 60, 70, or 75% of the total recombinant β-lactoglobulin proteins in the composition, preferably from about 50 to about 85%.

In one embodiment the modified β-lactoglobulin protein or the composition comprises one or more, 2 or more, 3 or more or 4 or more proteins comprising or consisting of a sequence selected from the group comprising SEQ ID Nos: 7-12, and 14-16.

In one embodiment
a) the amount of modified β-lactoglobulin protein is at least about 45, 50 or 60% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of a wild type, mature β-lactoglobulin, preferably SEQ ID No:2, is at least about 1, 2 or 3% or 5% of the total recombinant β-lactoglobulin proteins in the composition, preferably from about 1 to about 15%; and
c) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:2, is at least about 40, 45, 50, 55, or 60% of the total recombinant β-lactoglobulin proteins in the composition, preferably from about 40 to about 70%.

In one embodiment the modified β-lactoglobulin protein or the composition comprises one or more, 2 or more, 3 or more or 4 or more proteins comprising or consisting of a sequence selected from the group comprising SEQ ID Nos: 22-26, 28 and 29.

In one embodiment
a) the amount of modified β-lactoglobulin protein is at least about 70, 75 or 80% of the total recombinant β-lactoglobulin proteins in the composition; and
b) the amount of modified β-lactoglobulin protein comprising or consisting of an amino acid sequence that comprises an N-terminal truncation of from about 1 to about 20 amino acids relative to the sequence of a wild type, mature β-lactoglobulin, preferably SEQ ID No:2, is at least about 70, 75 or 80% of the total recombinant β-lactoglobulin proteins in the composition, preferably from about 70 to about 90%.

In one embodiment the modified β-lactoglobulin protein or the composition comprises one or more, 2 or more, 3 or more or 4 or more proteins comprising or consisting of a sequence selected from the group comprising SEQ ID Nos: 23-26, 28 and 61.

In various embodiments the composition comprises the recombinant β-lactoglobulin proteins of:
a) SEQ ID Nos: 1, 4, 5 and 6;
b) SEQ ID Nos: 1, 71 and 72; or
c) SEQ ID No: 7.

In various embodiments the composition comprises the recombinant β-lactoglobulin proteins of:
a) SEQ ID Nos: 2, 19, 20 and 21;
b) SEQ ID Nos: 2, 73 and 74; or
c) SEQ ID No:22.

In various embodiments the composition comprises first, second and third recombinant, elongated β-lactoglobulin proteins, wherein
a) the first recombinant, elongated β-lactoglobulin protein has an N-terminal elongation of sequence EAEAM;
b) the second recombinant, elongated β-lactoglobulin protein has an N-terminal elongation of sequence EAM; and
c) the third recombinant, elongated β-lactoglobulin protein has an N-terminal elongation of sequence M.

In various embodiments the composition comprises a recombinant, elongated β-lactoglobulin protein having an N-terminal elongation of sequence R.

In various embodiments the composition comprises a first and a second recombinant, elongated β-lactoglobulin proteins, wherein
a) the first recombinant, elongated β-lactoglobulin protein has an N-terminal elongation of sequence EAEA; and
b) the second recombinant, elongated β-lactoglobulin protein has an N-terminal elongation of sequence EA.

In the specification, where the amount of modified β-lactoglobulin protein or proteins is provided relative to the total recombinant β-lactoglobulin protein in the composition, this is an approximated amount of protein based upon the ionization signal intensity as determined by an LC-MS analysis of the total recombinant β-lactoglobulin proteins. The LC-MS analysis may be performed by any suitable method known in the art, for example, the method described herein in the examples.

### Properties of the Protein Compositions

In some embodiments, the compositions of the invention have acceptable or improved nutritional value and/or protein quality compared with wild type β-lactoglobulin proteins. In some embodiments a given nutritional value or protein digestibility is achieved by substituting one or more amino acids in the sequence of a wild type, mature β-lactoglobulin sequence with one or more amino acids, especially one or more essential amino acids.

In various embodiments the plurality of recombinant β-lactoglobulin proteins comprises at least one modified β-lactoglobulin protein comprising or consisting of an amino acid sequence that comprises one or more substitutions of from about 1 to about 20 amino acids relative to the amino acid sequence of any one of a) a wild type, mature β-lactoglobulin; b) SEQ ID Nos:1 to 3, 34, 36, 38, 40, 42, 44, 45, or 47; c) SEQ ID Nos 1 to 3, or d) SEQ ID No 1 or 2, wherein the one or more substitutions comprising one or more essential amino acids.

In various embodiments the essential amino acids are selected from the group consisting of histidine, isoleucine, leucine, lysine, methionine, phenylalanine, threonine, tryptophan and valine. In an exemplary embodiment the essential amino acid is histidine.

In various embodiments the one or more substitutions comprises at least 1, 2, 3, 4, 5, 6, 7, 8, 9, 10, 15 or 20 essential amino acids, or from about 1 to about 20, about 1 to about 10, or about 1 to about 5 essential amino acids

Methods of assessing protein quality are well known in the art. One such method is to calculate the Protein Digestibility-Corrected Amino Acid Score (PDCAAS). More recently, the PDCAAS has been replaced with the Digestible Indispensable Amino Acid Score (DIAAS). Both methods evaluate the quality of a protein based on both the amino acid requirements of humans and their ability to digest protein. DIAAS can be calculated as described by FAO (FAO. FOOD AND. NUTRITION. PAPER. 92. Dietary protein quality evaluation in human nutrition. Report of an. FAO Expert Consultation. 31 Mar.-2 Apr. 2011).

Wild type bovine β-lactoglobulin (all variants) have a mean DIAAS score of 0.91.

In various embodiments the plurality of recombinant β-lactoglobulin proteins in the compositions described herein have a mean DIAAS score of at least 0.8, 0.85, 0.88, 0.89, 0.9, 0.91, 0.92 or 1. In some embodiments the plurality of recombinant proteins has a mean DIAAS score equal to or greater than 0.91.

In some embodiments, it is desirable that the plurality of recombinant proteins exhibits improved resistance to heat denaturation. Accordingly, in various embodiments the plurality of recombinant proteins denatures at a higher temperature than wild type bovine β-lactoglobulin A or B as measured by differential scanning calorimetry. In other embodiments, it is desirable that the plurality of recombinant proteins exhibits improved resistance to heat denaturation. Accordingly, in various embodiments the plurality of recombinant proteins denatures at a lower temperature than wild type bovine β-lactoglobulin A or B as measured by differential scanning calorimetry.

Differential scanning calorimetry may be performed using any suitable method known in the art. Exemplary methods include those described in Ruegg et al., 1977, J Dairy Res, 44(3): p. 509-520 and Paulsson, et al., 1985, Thermochimica Acta, 95(2): p. 435-440.

In some embodiments, the composition forms an improved heat set gel compared with that formed with wild type bovine β-lactoglobulin A or B. Accordingly, in various embodiments the storage modulus of a heat set gel comprising the composition at a pH of about 7 is at least equivalent to that of NZMP SureProtein™ WPI895 when measured at a frequency of 1 Hz and at 20° C., wherein the heat set gel is formed by heating a solution comprising 10% by weight of the plurality of recombinant proteins by heating from 20 to 80° C. at a rate of 1° C./min, holding the solution at 80° C. for 30 min, and cooling the solution from 80 to 20° C. at a rate of 1° C./min and holding the temperature at 20° C. for 20 min on a rheometer to form the gel. The storage modulus of a heat set gel is determined during a temperature cycle, which involves holding a samples at 20° C. for 10 min, followed by a temperature ramp from 20° C. to 80° C. at a rate of 1° C./min, followed by a holding step at 80° C. for 30 min, followed by a temperature ramp from 80° C. to 20° C. at a rate of 1° C./min and finally a holding step at 20° C. for 20 min. The G′ is reported at the end of the temperature cycle.

In various embodiments the stiffness (G′) at 20° C. of a heat set gel comprising the composition at a pH of about 7 is at least 7,000 when measured at a frequency of 0.1 Hz and a strain of 0.25%, wherein the heat set gel is formed by heating a solution comprising 10% by weight of the protein(s) from 20° C. to 90° C. at a rate of 5° C./min, holding at 90° C. for 30 min, and cooling to 20° C. at a rate of 5° C./min. For example, the stiffness (G′) may be at least 8,000, at least 9,000, at least 10,000, at least 11,000, at least 12,000, at least 13,000, at least 14,000, at least 15,000, at least 16,000, at least 17,000, at least 18,000, at least 19,000, at least 20,000, at least 21,000, at least 22,000, at least 23,000, at least 24,000, at least 25,000, at least 26,000, at least 27,000, at least 28,000, at most 30,000, such as at most 29,000, at most 28,000, at most 27,000, at most 26,000, at most 25,000, at most 24,000, at most 23,000, at most 22,000, at most 21,000, at most 20,000, at most 19,000, at most 18,000, at most 17,000, at most 16,000, at most 15,000, at most 14,000, at most 13,000, at most 12,000, at most 11,000, at most 10,000, at most 9,000, or at most 8,000, and ranges may be chosen between any of these values such as from 8,000 to 16,000, from 8,000 to 15,000, from 8,000 to 14,000, from 8,000 to 13,000, from 8,000 to 12,000, from 8,000 to 11,000, from 8,000 to 10,000, from 9,000 to 16,000, from 9,000 to 15,000, from 9,000 to 14,000, from 9,000 to 13,000, from 9,000 to 12,000, from 9,000 to 11,000, from 9,000 to 10,000, from 20,000 to 30,000, from 21,000 to 30,000, from 22,000 to 30,000, from 23,000 to 30,000, from 24,000 to 30,000, from 25,000 to 30,000, from 26,000 to 30,000, from 27,000 to 30,000, from 28,000 to 30,000, or from 28,000 to 29,000.

In various embodiments the stiffness (G′) at 20° C. of a heat set gel comprising the composition at a pH of about 7 is at least about 80% of that of a heat set gel formed with wild type bovine β-lactoglobulin, when measured at a frequency of 0.1 Hz and a strain of 0.25%, wherein the heat set gel is formed by heating a solution comprising 10% by weight of the protein(s) from 20° C. to 90° C. at a rate of 5° C./min, holding at 90° C. for 30 min, and cooling to 20° C. at a rate of 5° C./min. For example, the stiffness (G′) may be at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at most about 200%, at most about 195%, at most about 190%, at most about 185%, at most about 180%, at most about 175%, at most about 170%, at most about 165%, at most about 160%, at most about 155%, at most about 150%, at most about 145%, at most about 140%, at most about 135%, at most about 130%, at most about 125%, at most about 120%, at most about 115%, at most about 110%, at most about 105%, at most about 100%, or at most about 95% of that of a heat set gel formed with wild type bovine β-lactoglobulin, and ranges may be chosen between any of these values such as from 85% to 180%, from 85% to 170%, from 85% to 160%, from 85% to 155%, from 85% to 150%, from 85% to 145%, 90% to 180%, from 90% to 170%, from 90% to 160%, from 90% to 155%, from 90% to 150%, from 90% to 145%, 100% to 180%, from 100% to 170%, from 100% to 160%, from 100% to 155%, from 100% to 150%, from 100% to 145%, from 150% to 200%, from 160% to 200%, from 170% to 200%, from 180% to 200%, from 190% to 200%, from 150% to 190%, from 160% to 190%, from 170% to 190%, or from 180% to 190%.

In some embodiments, the composition forms an improved acid set gel compared with that formed with wild type bovine β-lactoglobulin A or B. Accordingly, in various embodiments an acid-set gel formed by combining milk and 1.0% by weight of the protein(s), heating to 80° C. for 30 minutes, cooling to 30° C., and acidifying with 2% w/w glucono-δ-lactone (GDL) has an increased final stiffness compared with an acid-set gel formed using wild type bovine β-lactoglobulin.

In various embodiments, an acid-set gel formed by combining milk and 1.0% by weight of the protein(s), heating to 80° C. for 30 minutes, cooling to 30° C., and acidifying with 2% w/w glucono-δ-lactone (GDL) has a final stiffness of at least 400 Pa, such as at least 410 Pa, at least 420 Pa, at least 430 Pa, at least 440 Pa, at least 450 Pa, at least 460 Pa, at least 470 Pa, at least 480 Pa, at least 490 Pa, at least 500 Pa, at least 520 Pa, at least 540 Pa, at least 560 Pa, at least 580 Pa, at least 600 Pa, at least 620 Pa, at least 640 Pa, at least 660 Pa, at least 680 Pa, at least 700 Pa, at least 720 Pa, at least 740 Pa, at least 760 Pa, at least 780 Pa, or at least 800 Pa. In various embodiments, an acid-set gel formed by combining milk and 1.0% by weight of the protein(s), heating to 80° C. for 30 minutes, cooling to 30° C., and acidifying with 2% w/w glucono-δ-lactone (GDL) has a final stiffness of at least 100% of that of an acid-set gel formed using wild type bovine β-lactoglobulin, such as at least 110%, at least 120%, at least 130%, at least 140%, at least 150%, at least 160%, at least 170%, at least 180%, at least 190%, or at least 200% of that of an acid-set gel formed using wild type bovine β-lactoglobulin.

During recombinant protein production, aspartyl proteases derived from the host cells are typically not entirely removed by purification. Advantageously, in some embodiments described herein the compositions may be substantially free of aspartyl protease-like activity.

Such embodiments may confer certain advantages when used as functional ingredients in foods due to the avoidance of undesirable gelling caused by unwanted proteolysis of casein, particularly κ-casein, in foods.

Aspartyl proteases (also known as aspartic proteases) EC 3.4.23 are a catalytic type of protease enzyme that utilise an activated water molecule bound to one or more aspartate residues for catalysis of peptide substrates. Most aspartyl proteases have two highly conserved aspartates in the active site of the enzyme and are optimally active at acidic pH.

In various embodiments the compositions described herein may comprise an aspartyl protease having a sequence of SEQ ID No: 62 or 63.

The term “substantially free of aspartyl protease-like activity” as used herein refers to a composition that, when added to casein, in particular κ-casein, does not result in degradation of the casein and/or κ-casein. In some embodiments this term refers to a composition that, when added to casein, does not result in degradation of αs-casein, β-casein, κ-casein or any combination of any two or more thereof. In various embodiments, no degradation of κ-casein is observed and/or no production of para-κ-casein is observed when the compositions described herein are incubated with a substrate comprising κ-casein. In some embodiments, when the composition is added to κ-casein, less than about 45%, 40%, 35%, 30%, 25%, 20%, 15%, 10%, 5%, 1% or 0.5% of the κ-casein is degraded to para-κ-casein. In various embodiments, when the composition is added to a skim milk composition comprising 10% by weight total solids to a concentration of the one or more recombinant proteins of about 1% by weight and incubated at ambient temperature for 24 hours, less than about 45%, 40%, 35%, 30%, 25%, 20%, 15%, 10%, 5%, 1% or 0.5% of κ-casein in the skim milk composition is degraded to form para-κ-casein.

Methods for detecting aspartyl protease activity are known in the art and can be used with recombinant protein compositions. One such method which can be used to measure aspartyl protease activity via κ-casein degradation is ‘lab-on-a-chip” sodium dodecyl sulphate poly acrylamide gel electrophoresis (SDS-PAGE) as described herein in the examples.

Briefly a recombinant protein composition is added to a solution of skim milk solids, incubated for a period of time and then run on either an SDS gel or “lab on chip” to isolate and quantify the amount of κ-casein and its degradation product para-κ-casein. Detailed methods for this process can be found in “The use of “lab-on-a-chip” microfluidic SDS electrophoresis technology, S. G. Anema 2009, International Dairy Journal 19 (2009) 198-204”.

In some embodiments wherein the composition is substantially free of aspartyl protease-like activity, when the composition is added to a skim milk composition comprising 10% by weight total solids to a concentration of the one or more recombinant proteins of about 1% by weight and the skim milk composition is held at ambient temperature for 15 minutes then heated at 140° C., the composition has not coagulated after about 14, 15, 16, 17, 18, 19, 20, 25 or 30 minutes. The time to coagulation can be tested according to various methods known in the art, including those described herein. Time to coagulation can be measured by adding the recombinant milk protein composition to skim milk, incubating for a certain period of time and then heating to a selected temperature.

In some embodiments wherein the composition is substantially free of aspartyl protease-like activity, when the composition is added to a skim milk composition comprising 10% by weight total solids to a concentration of the one or more recombinant proteins of about 1% by weight, and the skim milk composition is heated at 40° C. for 12 hours, the skim milk composition does not reach gelation point. Gelation can be measured by various methods known in the art, including those as described herein. Briefly, a protein composition is added to skim milk and the sample is placed on a rheometer. Temperature is increased and the rheological properties of the sample are continuously monitored over a period of time. Gelation point can be detected by a change in the G′ from baseline.

### 2. Recombinant Expression

The recombinant β-lactoglobulin proteins in the compositions of the invention are produced by recombinant expression in a host cell. As used herein, a “host” or “host cell” denotes any protein production host selected or genetically modified to produce a desired product. Exemplary hosts include fungi, such as filamentous fungi, as well as bacteria, yeast, algae, plant, insect, and mammalian cells.

In various embodiments, the host cell is a yeast cell selected from the list consisting of Pichia pastoris (also known as Komagataella phaffii), Kluyveromyces lactis, Saccharomyces cerevisiae, Schizosaccharomyces pombe, Yarrowia lipolytica, Candida glabrata, Ashbya gossypii, Cyberlindnera jadinii, Pichia methanolica, Hansenula polymorpha, Candida boidinii, Arxula adeninivorans, Xanthophyllomyces dendrorhous, and Candida albicans species. In some embodiments, the yeast cell is a Saccharomycete.

In some embodiments, the host cell is a fungal cell selected from the list consisting of Aspergillus spp. and Trichoderma spp.

In various embodiments the host cell is selected from Pichia pastoris, Kluyveromyces lactis and Aspergillus niger. In various embodiments the host cell is selected from Pichia pastoris, Kluyveromyces lactis, Saccharomyces cerevisiae and Aspergillus niger. In other embodiments the host cell is selected from Pichia pastoris and Aspergillus niger. In other embodiments the host cell is selected from Pichia pastoris, Kluyveromyces lactis and Saccharomyces cerevisiae.

In some embodiments, the host cell may be a bacterial host cell such as Lactococcus lactis, Bacillus subtilis or Escherichia coli. Other host cells include bacterial host such as, but not limited to, Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum and Synechocystis sp. 6803.

In various embodiments the composition may comprise a plurality of recombinant β-lactoglobulin proteins produced separately by one or more different host cells of differing microbial species, genera or strains of the same species. Combining modified recombinant β-lactoglobulin proteins produced by different species may provide certain advantages, for example, to achieve a complement of modified recombinant β-lactoglobulin proteins to achieve a particular desired functionality of the composition or to achieve desired production volumes.

In some embodiments the composition may comprise one or more recombinant β-lactoglobulin proteins expressed by a first host cell and one or more recombinant β-lactoglobulin proteins expressed by a second host cell to form the plurality of recombinant β-lactoglobulin proteins. In various embodiments, the second host cell may be a different species to the first host cell or the second host cell may be a different strain of the same species as the first host cell. In various embodiments the composition may further comprise one or more recombinant β-lactoglobulin proteins expressed by a third and/or fourth host cell, and so on.

Host cells comprising genetic constructs, such as expression constructs, as disclosed herein may be used in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of proteins disclosed herein. Such methods may involve the culture of host cells in an appropriate medium in conditions suitable for or conducive to expression of a polynucleotide or protein disclosed herein. The expressed recombinant proteins, which may optionally be secreted into the culture, may then be separated from the medium, host cells or culture medium by methods well known in the art (e.g. Deutscher, Ed, 1990, Methods in Enzymology, Vol 182, Guide to Protein Purification).

Expression of a target protein can be provided by an expression vector, a plasmid, a nucleic acid integrated into the host genome or other means. For example, in some embodiments the host cell comprises a polynucleotide, e.g. an expression vector that comprises a sequence encoding a β-lactoglobulin protein (x) and may additionally comprise one or more of (a) a promoter element, (b) a signal peptide sequence, (c) a leader sequence, (d) a processing site and (e) a terminator element.

The promoter (a) and leader sequence (c) may be, or may be derived from, any suitable yeast, fungal, bacterial or mammalian promoter or leader sequence.

In various embodiments the host cell comprises a polynucleotide, such as an expression vector, comprising a processing site located between a leader sequence and the sequence encoding a mature dairy protein. In various embodiments the processing site may be a KEX processing site. In various embodiments the processing site may have the sequence KREA, KREAEA, KR or KREAEAM.

Expression vectors that can be used for expression of the dairy protein include those containing an expression cassette with elements (a), (b), (c), (d) and/or (e). In some embodiments, the signal peptide sequence (b) need not be included in the vector. In some cases, a signal peptide may be part of the native signal sequence of the protein, for instance, the protein may comprise a native signal sequence as bolded in SEQ ID NOs: 31 to 33, 35, 37, 39, 41, 43, 46, and 48. In some cases, the vector comprises a polynucleotide encoding a protein sequence as exemplified in SEQ ID NOs: 1 to 3 and 31 to 48. In some cases, the vector may comprise a polynucleotide encoding a mature protein sequence, as exemplified in SEQ ID NOs: 1 to 3, 34, 36, 38, 40, 42, 44, 45, and 47 with a heterologous signal sequence. In general, the expression cassette is designed to mediate the transcription of the transgene when integrated into the genome of a cognate host microorganism or when present on a plasmid or other replicating vector maintained in a host cell.

To aid in the amplification of the vector prior to transformation into the host microorganism, a replication origin (f) may be contained in the vector. To aid in the selection of microorganism stably transformed with the expression vector, the vector may also include a selection marker (g). The expression vector may also contain a restriction enzyme site (h) that allows for linearization of the expression vector prior to transformation into the host microorganism to facilitate the expression vectors stable integration into the host genome. The expression vector may contain integration sequences (i) homologous to genomic sequences of the host cell that enable or aid integration of a fragment of the expression vector or the entire expression vector into the genome of the host cell. In some embodiments the expression vector may contain any subset of the elements (a) to (i). Other expression elements and vector element known to one of skill in the art can be used in combination or substituted for the elements described herein. For example, because many microorganisms are capable of expressing multiple gene products from a polycistronic mRNA, multiple polypeptides can be expressed under the control of a single regulatory region for those microorganisms, if desired. These might include one or more β-lactoglobulin proteins, one or more different dairy proteins (for example, one or more caseins, α-lactalbumin or lactoferrin proteins), and/or one or more non-dairy proteins.

Gram positive bacteria (such as Lactococcus lactis and Bacillus subtilis) may be used to secrete target proteins into the media, and gram-negative bacteria (such as Escherichia coli) may be used to secrete target proteins into periplasm or into the media. In some embodiments, the bacterially-expressed proteins expressed may not have any post-translational modifications (PTMs), which means they are not glycosylated and/or may not be phosphorylated.

Target β-lactoglobulin proteins may be expressed and produced in L. lactis both in a nisin-inducible expression system (regulated by PnisA promoter), lactate-inducible expression system (regulated by P170 promoter) or other similar inducible systems, as well as a constitutively expressed system (regulated by P secA promoter), wherein both are in a food-grade selection strain, such as NZ3900 using vector pNZ8149 (lacF gene supplementation/rescue principle). The secretion of functional proteins may be enabled by the signal peptide of Usp45 (SP(usp45)), the major Sec-dependent protein secreted by L. lactis.

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3rd edition), Cold Spring Harbor Laboratory, Cold Spring Harbor Laboratory Press, or F. Ausubel et al, eds., “Current protocols in molecular biology”, Green Publishing and Wiley Interscience, New York (1987).

In some embodiments where it is desirable that the recombinant protein composition is substantially free of aspartyl protease-like activity, host cells having functional knockout or replacement of the endogenous pep4 gene in the host cell may be used. Suitable methods for achieving functional knockout or gene replacement in a host cell are well known in the art, and include CRISPR/CAS9 technology, mutation of the coding sequence to modify the amino acid sequence of the protein encoded by the gene to render it non functional, or by deletion and/or insertion of nucleotides into the coding sequence. Alternatively, the promoter expressing the pep4 gene can be replaced by another promoter with low regulated expression.

In one embodiment, the functional knockout/gene replacement of pep4 and insertion of an expression cassette for the one or more recombinant milk proteins may be accomplished simultaneously using CRISPR-CAS9 technology. An example is described herein in the examples. Briefly, CRISPR-CAS9 may be used to integrate a fragment containing the one or more recombinant milk proteins and deleting the pep4 gene in the host cell. An “all in one” expression vector may be used, which expresses both CAS9 and guide RNA (gRNA) targeting the pep4 gene sequence. In various embodiments the CAS9 gene and the pep4 gRNA are under the control of separate promoters. Preferably, the plasmid contains a selectable marker, such as an antibiotic selectable marker and an autonomously replicating sequence to select and maintain the plasmid in the cell after transformation to Pichia.

Compositions comprising the plurality of recombinant β-lactoglobulin proteins may be produced by culturing a host cell expressing the protein using standard methods well known in the art. The cell culture biomass may then be centrifuged to remove solid matter and the light phase subjected to filtration.

### 3. Food Products

The invention also relates to food products comprising a recombinant, elongated β-lactoglobulin protein having at least 70% sequence identity to a wild type, mature β-lactoglobulin, and methods of producing such food products.

In various embodiments, the food product may comprise at least about 1%, 1.5%, 2%, or 2.5% of the recombinant, elongated β-lactoglobulin protein by weight. In various embodiments, the food product may comprise from about 1 to about 50%, about 1 to about 45%, about 1 to about 40%, 1 to about 35%, about 1 to about 30%, or about 1% to about 25% of the recombinant, elongated β-lactoglobulin protein by weight, and useful ranges may be selected from between any of these values (for example, from about 1% to about 20%, or about 1% to about 16%, 1% to about 15%, 1% to about 14%, or about 1% to about 12%, or about 1% to about 10%, or about 2% to about 20%, or about 2% to about 16%, 2% to about 15%, 2% to about 14%, or about 2% to about 12%, or about 2% to about 10%, about 4% to about 20%, or about 4% to about 16%, 4% to about 15%, 4% to about 14%, or about 4% to about 12%, or about 4% to about 10%, about 5% to about 20%, or about 5% to about 16%, 5% to about 15%, 5% to about 14%, or about 5% to about 12%, or about 5% to about 10%).

In some embodiments, the food product comprises a composition described herein. The composition or the recombinant, elongated β-lactoglobulin protein may be mixed with one or more additional ingredients to produce a food product. In various embodiments, the method may comprise mixing the composition or the recombinant, elongated β-lactoglobulin protein with one or more additional ingredients to produce a food product. The food product may be any edible consumer product which is able to carry protein.

In various embodiments, the food product may comprise at least about 1%, 1.5%, 2%, or 2.5% total protein by weight. In various embodiments, the food product may comprise from about 1 to about 50%, about 1 to about 45%, about 1 to about 40%, 1 to about 35%, about 1 to about 30%, or about 1% to about 25% total protein by weight, and useful ranges may be selected from between any of these values (for example, from about 1% to about 20%, or about 1% to about 16%, 1% to about 15%, 1% to about 14%, or about 1% to about 12%, or about 1% to about 10%, or about 2% to about 20%, or about 2% to about 16%, 2% to about 15%, 2% to about 14%, or about 2% to about 12%, or about 2% to about 10%, about 4% to about 20%, or about 4% to about 16%, 4% to about 15%, 4% to about 14%, or about 4% to about 12%, or about 4% to about 10%, about 5% to about 20%, or about 5% to about 16%, 5% to about 15%, 5% to about 14%, or about 5% to about 12%, or about 5% to about 10%).

In various embodiments the food product may be a fermented food, a yoghurt, a soup, a sauce, a bar, a gel, a foam, a nutritional formulation, a beverage, a beverage whitener, a cheese, a dairy tofu, a food emulsion or a dessert.

In various embodiments the food product may be a yoghurt, drinking yoghurt, a bar, a gel, a foam, a nutritional formulation, medical food, dairy beverage, a product that requires the protein to form a heat-set gel, an acid protein fortified beverage, a jelly drink, a protein water, a foam, a heat-set foam extruded food product, or a food emulsion.

In various embodiments the food product is free of animal-derived ingredients. In various embodiments the food product is considered suitable for those on a vegan diet. The food product may comprise one or more additional sources of protein, including the examples described herein. In various embodiments the additional source of protein is a non-dairy source of protein, such as a plant protein described herein or algal protein or mycoprotein.

Liquid nutritional compositions may include a medical beverage. A beverage may include a sports beverage, dairy beverage, or a yoghurt beverage.

In various embodiments the food product has one or more characteristics of a dairy food product. In various embodiments the food product has one or more characteristics of a dairy food product selected from the group comprising: appearance, consistency, firmness, organoleptic properties, density, stiffness, structure, viscosity, texture, elasticity, storage stability, heat stability, acid-heat stability, coagulation, binding, leavening, aeration, foaming capacity, foam stability, foam overrun, behaviour when whipped, creaminess, gelling structure and emulsification.

In various embodiments the organoleptic properties are taste, aroma, mouthfeel, in-mouth creaminess, appearance, colour, grittiness, sandiness, and smoothness.

In various embodiments the food product may contain nutrients that include vitamins and minerals. The recommended daily requirements of vitamins and minerals can be specified for various population subgroups. See for instance, Dietary Reference Intakes: RDA and AI for vitamins and elements, United States National Academy of Sciences, Institute of Medicine, Food and Nutrition Board (2010) tables recommended intakes for infants 0-6, 6-12 months, children 1-3, and 4-8 years, adults males (6 age classes), females (6 age classes), pregnant (3 age classes) and lactating (3 age classes). Concentrations of essential nutrients in the liquid nutritional composition can be tailored in the exemplary serve size for a particular subgroup or medical condition or application so that the nutrition and ease of delivery requirements can be met simultaneously.

In various embodiments, the pH of the food product may be adjusted using food-safe acidic or basic additives. In various embodiments, the pH of the protein containing food product may be adjusted to about pH 3 to about pH 8, for example about pH 3.3 to about pH 8, about pH 4 to about pH 8, about pH 4 to about pH 7, or about pH 4 to about pH 6.8, or about pH 5 to about pH 7, or about pH 5 to about pH 6.8. In various embodiments, the pH of the protein containing food product may be adjusted to about pH 6.8.

pH may be measured by equilibrating samples to 25° C. and measuring using a pH probe (EC620132, Thermo Scientific) after calibrating using standards at pH 4, 7, and 10 (Pronalys, LabServ). Other methods of measuring pH will be apparent to a skilled worker.

In various embodiments the food product may be administered to a subject to maintain or increase muscle protein synthesis, maintain or increase muscle mass, prevent or increase loss of muscle mass, maintain or increase growth, prevent or decrease muscle catabolism, prevent or treat cachexia, prevent or treat sarcopenia, increase rate of glycogen resynthesis, modulate blood sugar levels, increase insulin response to raised blood glucose concentration, increase satiety, increase satiation, increase food intake, increase calorie intake, improve glucose metabolism, increase rate of recovery following surgery, increase rate of recovery following injury, increase rate of recovery following exercise, increase sports performance, and/or provide nutrition.

In various embodiments, the food product may comprise at least about 0.1% fat by weight, such as about 0.1%, or about 0.5%, or about 1%, or about 3%, or about 5%, or about 10% fat by weight. In various embodiments, the protein containing food product may comprise from about 0.1% to 40% fat by weight, and useful ranges may be selected from between any of these values (for example, from about 0.1% to about 40%, or about 0.5% to about 40%, or about 1% to about 40%, or about 3% to about 40%, or about 5% to about 40%, or about 10% to about 40%, or about 15% to about 40%, or about 20% to about 40%, or about 0.1% to about 35%, or about 0.5% to about 35%, or about 1% to about 35%, or about 3% to about 35%, or about 5% to about 35%, or about 10% to about 35%, or about 15% to about 35%, or about 20% to about 35%, or about 0.1% to about 30%, or about 0.5% to about 30%, or about 1% to about 30%, or about 3% to about 30%, or about 5% to about 30%, or about 10% to about 30%, or about 15% to about 30%, or about 20% to about 30%, or about 0.1% to about 20%, or about 0.5% to about 20%, or about 1% to about 20%, or about 3% to about 20%, or about 5% to about 20%, or about 10% to about 20%, or about 15% to about 20%).

In various embodiments, the food product may comprise at least about 0.1% carbohydrate by weight, such as about 0.1%, or about 0.5%, or about 1%, or about 3%, or about 5%, or about 10% fat by weight. In various embodiments, the protein containing food product may comprise from about 0.1% to 40% carbohydrate by weight, and useful ranges may be selected from between any of these values (for example, from about 0.1% to about 40%, or about 0.5% to about 40%, or about 1% to about 40%, or about 3% to about 40%, or about 5% to about 40%, or about 10% to about 40%, or about 15% to about 40%, or about 20% to about 40%, or about 0.1% to about 35%, or about 0.5% to about 35%, or about 1% to about 35%, or about 3% to about 35%, or about 5% to about 35%, or about 10% to about 35%, or about 15% to about 35%, or about 20% to about 35%, or about 0.1% to about 30%, or about 0.5% to about 30%, or about 1% to about 30%, or about 3% to about 30%, or about 5% to about 30%, or about 10% to about 30%, or about 15% to about 30%, or about 20% to about 30%, or about 0.1% to about 20%, or about 0.5% to about 20%, or about 1% to about 20%, or about 3% to about 20%, or about 5% to about 20%, or about 10% to about 20%, or about 15% to about 20%).

In various embodiments, the food product, preferably a medical beverage, may comprise at least about 10 kcal per 100 mL of the food product. In various embodiments, the protein containing food product may comprise from about 10 to about 400 kcal per 100 mL of the food product, and useful ranges may be selected from between any of these values (for example, from about 10 to about 400, 10 to about 350, or about 10 to about 300, or about 10 to about 300, or about 10 to about 250, or about 10 to about 200, or about 10 to about 150, or about 10 to about 100, or about 50 to about 400, or about 50 to about 350, or about 50 to about 300, or about 50 to about 300, or about 50 to about 250, or about 50 to about 200, or about 50 to about 150, or about 50 to about 100, or about 100 to about 400, or about 100 to about 350, or about 100 to about 300, or about 100 to about 300, or about 100 to about 250, or about 100 to about 200, or about 100 to about 150, or about 150 to about 400, or about 150 to about 350, or about 150 to about 300, or about 150 to about 300, or about 150 to about 250, or about 200 to about 400, or about 200 to about 350, or about 200 to about 300, or about 200 to about 350).

In one embodiment the food product is in bar or solid moulded form. In various embodiments the bar further comprises one or more additional ingredients selected from one or more sweeteners, one or more additional protein sources, one or more stability enhancers (such as glucose syrup, glycerine, plasticisers (such as glycerine), one or more lipids and one or more lecithins.

In various embodiments a shelf-stable protein beverage comprising 3.5% by weight of the plurality of recombinant proteins exhibits no visible sedimentation after 4 weeks at ambient temperature. “No visible sedimentation” means that no sedimentation is observed when the beverage is viewed unaided by the human eye in natural light.

In various embodiments, a solution comprising the plurality of recombinant proteins has acceptable or improved whipping and/or foaming properties. In various embodiments a solution of neutral pH comprising 10% by weight of the plurality of recombinant proteins has an overrun of at least 500% after 10 minutes whipping, and/or wherein a foam produced by whipping a solution of neutral pH comprising 10% by weight of the plurality of recombinant proteins exhibits no serum leakage for at least about 5 minutes after whipping. Overrun may be measured according to the method described herein in the examples.

In various embodiments a solution of neutral pH comprising 2.5% by weight of the one or more recombinant proteins has a foam volume after 70 seconds whipping of at least 75 mL.

In various embodiments, a solution of neutral pH comprising 2.5% by weight of the one or more of recombinant proteins, when whipped for 70 seconds, produces a foam that retains at least 50% of its volume after 45 minutes, such as at least 55%, at least 60%, at least 65%, at least 70%, or at least 75% of its volume after 45 minutes.

In various embodiments the recombinant, elongated β-lactoglobulin protein or the compositions form an emulsion of similar stability to that achieved with wild type bovine β-lactoglobulin A or B, or an emulsion exhibiting increased or decreased stability compared with an emulsion formed with wild type bovine β-lactoglobulin A or B.

In various embodiments the recombinant, elongated β-lactoglobulin protein or the compositions form an emulsion of similar or increased emulsifying capacity to that achieved with wild type bovine β-lactoglobulin A or B, or a combination thereof.

To determine the performance of recombinant, elongated β-lactoglobulin protein or compositions described herein, emulsions may be formed using a high-shear mixer or homogeniser.

In various embodiments, an emulsion comprising the recombinant, elongated β-lactoglobulin protein or the composition exhibits increased, the same or reduced stability compared with an emulsion comprising a protein of SEQ ID No:1 or 2, wherein the emulsion is formed by combining the composition, using different levels of oil ranging from 2.5 to 17.5% and water, pre-homogenizing and homogenizing by sonication at 100% amplitude for 10 min, and measuring particle size using laser light scattering.

In various embodiments, an emulsion comprising the recombinant, elongated β-lactoglobulin protein or the composition exhibits higher, the same or lower emulsifying capacity (EC) compared with an emulsion comprising a protein of SEQ ID No:1 or 2, wherein the emulsion is formed by combining the recombinant, elongated β-lactoglobulin protein or the composition, using different levels of oil to give a range of oil to protein ratios and homogenizing and immediately measuring the electrical conductivity to determine EC.

In various embodiments, an emulsion comprising a composition with 0.5% w/w protein has an EC of at least 900 mL oil/g protein, such as at least 1,000, at least 1,100, at least 1,200, at least 1,300, or at least 1,400 mL oil/g protein. In various embodiments, an emulsion comprising a composition with 0.5% w/w protein has an EC of at least 90% of that of wild type β-lactoglobulin, such as at least 95%, at least 100%, at least 105%, at least 110%, at least 115%, at least 120%, or at least 125% of that of wild type β-lactoglobulin.

In various embodiments the emulsion activity index (EAI) of an emulsion comprising the recombinant, elongated β-lactoglobulin protein or the composition is increased, the same or decreased compared with an emulsion comprising wild type bovine β-lactoglobulin A or B. In various embodiments the emulsion activity index (EAI) of an emulsion comprising the recombinant, elongated β-lactoglobulin protein or the composition is increased, the same or decreased compared with an emulsion comprising a protein of SEQ ID No:1 or 2, wherein the emulsion is formed by preparing a solution comprising 0.5% protein, 20% soya oil and water and homogenizing the solution at 200 bar, and wherein the EAI is determined by measuring the specific surface area of the emulsion droplets using laser light scattering.

In some embodiments wherein the food product comprises a composition that is substantially free of aspartyl protease-like activity, the food product may not readily coagulate when a composition described herein is added to one or more additional ingredients to produce the food product. In a preferred embodiment, when the composition is added to one or more additional ingredients, the resulting mixture does not rapidly coagulate.

In some embodiments wherein the food product comprises a composition that is substantially free of aspartyl protease-like activity, the food product does not readily gel when a composition described herein is added to one or more additional ingredients to produce the food product. In a preferred embodiment, when the composition is added to one or more additional ingredients, the resulting mixture does not rapidly gel.

The above methods should be considered in no way limiting and suitable variations or alternatives will be apparent to those skilled in the art.

### EXAMPLES

### Example 1

This example describes the production of Pichia pastoris strains producing a plurality of recombinant β-lactoglobulin proteins, and purification of the proteins to produce compositions of the invention.

### 1. Preparation of β-Lactoglobulin A and β-Lactoglobulin B DNA Constructs

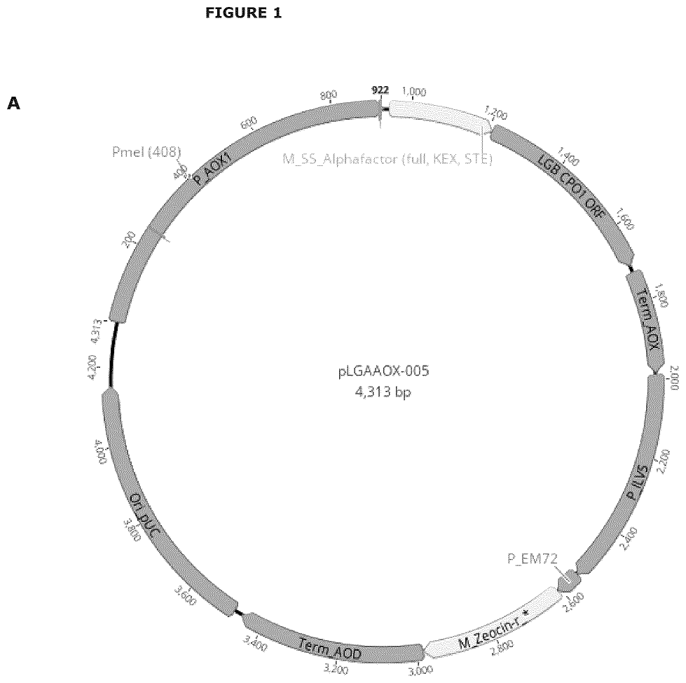

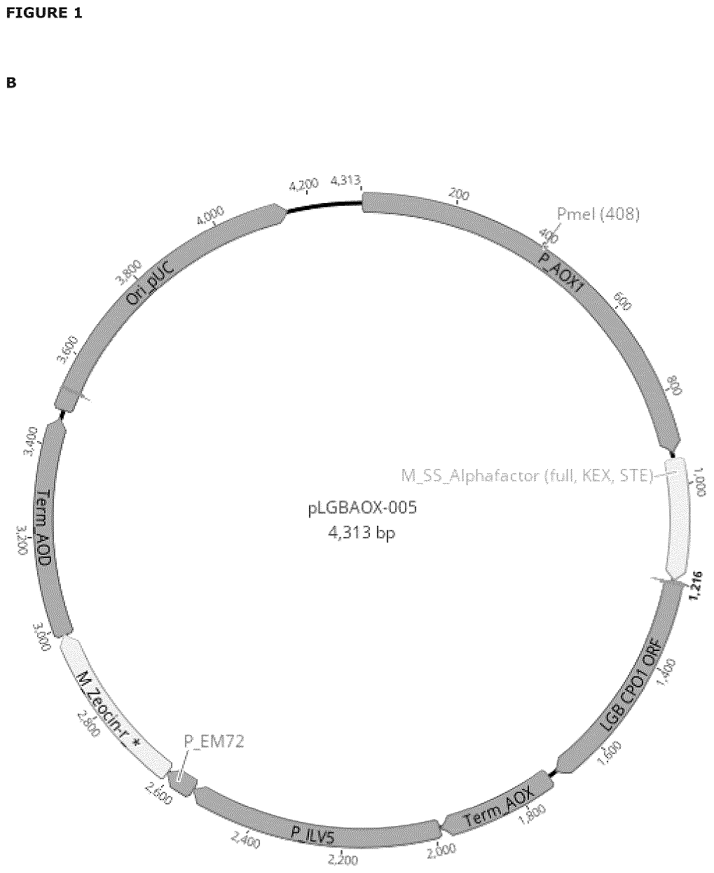

The proteins were expressed and produced using Pichia pastoris (currently renamed as Komagataella phaffii) host cells. Before transformation to the host cell, DNA constructs were designed and prepared as follows. The genes coding for wild type mature bovine β-lactoglobulin A and β-lactoglobulin B were provided to order by synthetic DNA provider ATUM (CA, USA). Codon usage was optimized for expression in Pichia pastoris. The β-lactoglobulin A and β-lactoglobulin B encoding sequences were fused behind the α-mating factor from S. cerevisiae followed by a Kex2 processing site (KREAEAM) composed of lysine, arginine (KR), a glutamine-alanine repeat (EAEA) and a methionine at the start of the β-lactoglobulin A and B amino acid sequence. This led to the synthesis of the two DNA sequences described as the gene coding for beta-lactoglobulin A (SEQ ID No: 49) and the gene for beta-lactoglobulin B (SEQ ID No:50). The genes were delivered cloned in the standard ATUM vector pD912 and by that placed under control of the methanol inducible AOX1 promoter. The delivered plasmid containing the beta-lactoglobulin A was named pLGAAOX-005 (SEQ ID No: 51) and the plasmid containing the beta-lactoglobulin B named pLGBAOX-005 (SEQ ID No: 52). Both plasmids are shown in FIG. 1.

2. Transformation of pLGAAOX-005 and pLGBAOX-005 to Pichia pastoris

The vectors pLGAAOX-005 and pLGBAOX-005 were digested with PmeI and Pichia pastoris strain NRRL-Y11430 (a wild type strain received from the ARS Culture Collection) was transformed with the digested DNA. Transformation procedure was performed according to condensed electroporation protocol using freshly prepared solutions (Lin-Cereghino J1, Wong W W, Xiong S, Giang W, Luong L T, Vu J, Johnson S D, Lin-Cereghino G P. Biotechniques. (2005) 38, (1):44-48). Transformants were plated on YPDS agar plates with 1000 μg/mL Zeocin (YPDS: 1% yeast extract, 2% peptone, 2% glucose, 1M sorbitol, 2% agar) and incubated at 30° C. for 72 h. Single colonies were picked from the plates and transferred to 96-well MTP agar plates containing YEPhD agar with 500 μg/ml. (YEPhD agar 10 g/l Yeast extract, 20 g/l Phytone peptone, 20 g/l glucose, 15 g/l Oxoid agar, Zeocin added after autoclaving). MTP agar plates were incubated for 72 hours at 30° C. From the MTP agar plate transformants were inoculated into 200 μl YephD medium in HDWP using a pin and incubated overnight in an INFORS MTP incubator at 30° C., 750 rpm and 80% humidity. This was used as pre-culture for the protein production experiment.

3. Production of recombinant β-lactoglobulin A and B in P. pastoris

The pre-culture was used to inoculate the production medium. From the 200 μl pre-culture, 20 μl was inoculated into 2 ml BMM medium (0.2 M Potassium Phosphate buffer pH 6.8, 13.4 g/l Yeast Nitrogen Base, 0.4 mg/l Biotin) in a 24-deepwell plate covered with a breathable seal. Incubation of the plate was done in an INFORS MTP incubator at 28° C., 550 rpm and 80% humidity for 3 days. At the start and after 24 hours and 48 hours 1% of methanol was added for growth of the methanol utilizing Pichia pastoris strains and induction of the AOX1 promoter expressing the beta-lactoglobulin A and B genes. After 72 hours the 24-deepwell plates were centrifuged and 200 μl supernatant of the sample for each strain was transferred to a microtiter plate. The supernatant was analysed by LC-MS.

Food grade protein compositions were produced using a 10 litre scale fermenter, followed by downstream processing including removal of biomass by centrifugation, ultrafiltration for concentration and dialysis, ion exchange for purification, ultrafiltration for concentration and dialysis for removal of salts and drying by freeze drying.

### 4. LC-MS Analysis Intact Protein

Beta-lactoglobulin A and B standards (Sigma L7880, L8005, respectively) and supernatants were analyzed with a Synapt G2-s (Waters) equipped with a Acquity I-Class LC (Waters). The chromatographic system consisted of a BEH C4 300A 1.7 um 2.1*50 column (Waters), which was kept at 75° C. and gradient elution using A: 0.1% Formic Acid in water, and B: Formic Acid 0.1% in Acetonitrile:Water 90:10 (v/v), and a flow-rate of 400 ul/min. The gradient started at 3% B for 2 minutes, then linear increasing to 17% B in 0.2 minutes, following by linear increasing to 66% B in 5.8 minutes, directly increasing to 95% and kept here for 1 minutes, then directly decreasing to 3% B and kept here for 5 minutes, for re-equilibrating the LC-MS system.

Standard, native (wild type) mature β-lactoglobulin A and B were used to test the intact protein LC-MS system. In Electrospray ionization mass spectrometry (ESI-MS), proteins are characterized by multiply-charged species.

With deconvolution software (Waters Maxent1) the average mass could be calculated. For both the beta-lactoglobulin A (Mavg=18363) and beta-lactoglobulin B (Mavg=18277) standards the correct average mass of the amino acid sequence including the 2 S-S bridges was obtained.

### Example 2

This example describes the production of Aspergillus niger strains expressing a plurality of recombinant β-lactoglobulin proteins, and purification of the proteins to produce recombinant β-lactoglobulin protein compositions.

1. Preparation of β-Lactoglobulin A and β-Lactoglobulin B DNA Constructs for A. niger

Methods for transformation, genetic modification etc of fungal host cells are known from e.g. EP-A-0 635 574, WO 98/46772, WO 99/60102 and WO 00/37671, WO90/14423, EP-A-0481008, EP-A-0635 574 and U.S. Pat. No. 6,265,186.

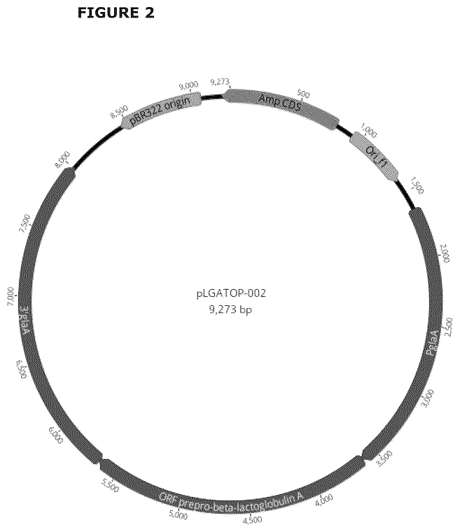

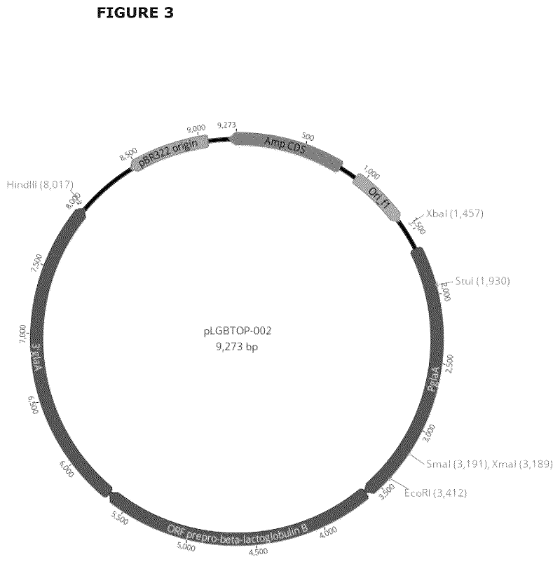

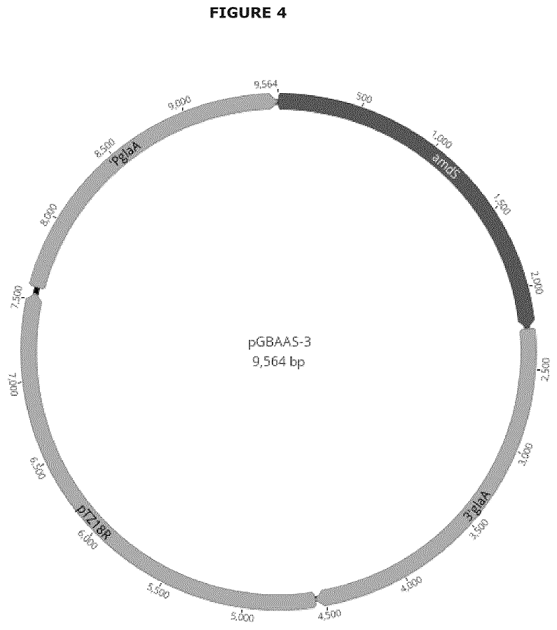

Before transformation to A. niger, DNA constructs were designed and prepared as follows. The genes coding for bovine β-lactoglobulin A and β-lactoglobulin B were ordered at synthetic DNA provider ATUM (CA, USA). Codon usage was optimized for expression in A. niger. The β-lactoglobulin A was cloned creating the plasmid pLGATOP-002 (SEQ ID No: 53) with the open reading frame comprising the glucoamylase signal sequence, inactive glucoamylase, Kex-02 processing site with amino acid sequence KREA and bovine β-lactoglobulin variant A. The DNA sequence for the open reading frame is provided as SEQ ID No: 54. The β-lactoglobulin B was cloned creating the plasmid pLGBTOP-002 (SEQ ID No: 55) with the open reading frame comprising glucoamylase signal sequence, inactive glucoamylase, Kex-02 processing site with amino acid sequence KREA and bovine beta-lactoglobulin variant B. The described DNA sequence for the open reading frame was listed as SEQ ID No: 56. The genes are expressed using the glaA promoter and glaA terminator. The plasmids pLGATOP-002 and pLGBTOP-002 containing the beta-lactoglobulin A and B expression constructs are shown in FIG. 2. Selection of transformants in A. niger was based on co-transformation with the pGBAAS3 marker plasmid (SEQ ID No:57), containing the amdS marker. The plasmid map of pGBAAS3 is shown in FIG. 3.

2. Transformation of pLGATOP-002 and pLGBTOP-003 to A. niger

Transformation experiments were performed with methods as described in WO199846772, WO199932617, WO2011009700, WO2012001169, WO2013135729, WO2014013073 and WO2014013074 and references therein.

The A. niger strain GBA309 used during transformation was derived from the Aspergillus niger wild-type strain deposited at the CBS Institute under the deposit number CBS 513.88. The construction of GBA309 as starting strain has been described in detail in WO2011/009700. The GBA306 strain has the following genotype: Ag/aA, ApepA, hdfA, an adapted BamYW amplicon, AamyBII, AamyBI, and amyA. The β-lactoglobulin A and B producing transformants were made by co-transformation with the amdS selectable marker-gene containing vector pGBAAS-3 using the method as described in WO2011/009700 and WO2012/001169. After transformation and counter-selection (as also described in WO98/46772 and WO99/32617), followed by selection of strains with multiple copies, a multi-copy beta-lactoglobulin A (named AN-02) was selected and a beta-lactoglobulin B producing strain (named AN-01) was selected.

3. Production of β-Lactoglobulin A and B Production in A. niger

Fresh A. niger spores containing the vectors described above were prepared and used for generating sample material by cultivation of the strains in 24-well plates. The spore solution was used to inoculate 4 ml CSM-MES in 24-well plates. CSM/MES medium consisted of: 150 g/l maltose*H₂O, 60 g/l Soytone (pepton), 1 g/l NaH₂PO₄*H₂O, 15 g/l NH₄SO₄, 1 g MgSO₄·7H₂O, 0.08 g/l Tween 80, 0.02 g/l Basildon (antifoam), 20 g/l MES, 1 g/l L-arginine. The ingredients were dissolved in demineralized water and the pH was adjusted to pH=6.2 with NaOH or H₂SO₄; sterilized for 15 minutes at 110° C., after which 10 ml of a solution containing 5000 IU/ml penicillin and 5 mg/ml streptomycin was added per liter media after cooling to room temperature.

Food grade protein compositions were produced as described in Example 1.

Cultures were incubated at 34° C. for 5 days. Supernatants were analysed and quantified by LC-MS using calibration curves of standards purchased from Sigma.

The strain AN-02 produced 908 mg/L beta-lactoglobulin A in MTP as quantified using LC-MS/MS according to the method described in Example 1. The strain AN-01 produced 1156 mg/L beta-lactoglobulin B in MTP as quantified using LC-MS/MS.

### Example 3

This example describes the production of Kluyveromyces lactis strains expressing a plurality of recombinant β-lactoglobulin proteins, and purification of the proteins to produce compositions of the invention.

1. Preparation of β-Lactoglobulin B DNA Constructs for K. lactis

The Kluyveromyces lactis strain GG799 was used as a wild-type starting strain. This strain was obtained from New England Biolabs, Ipswich, Massachusetts, USA. A K. lactis host cell strain was constructed with three copies of β-lactoglobulin B expression constructs. The 3 expression constructs differ in promoter and terminator sequence but have the same DNA sequence for the open reading frame (ORF). The ORF consists of the β-lactoglobulin B coding region fused to the full length 89 amino acids alpha-mating factor prepro sequence of K. lactis including the Kex2 processing site having the amino acid sequence KR. Expression cassette 1 consists of the ADH2 promoter, ORF and LAC4 terminator, expression cassette 2 consists of the PGK1 promoter ORF and BAR1 terminator and expression cassette 3 consists of the ENO1 promoter, ORF and PCR1 terminator. The 3 expression cassettes were integrated in the invertase locus, the complete sequence of this three expression cassette sequence is provided as SEQ ID No:28.

2. Transformation of the 3-Copy Construct into K. Actis

Transformation and strain selection were performed by electroporation and selection on acetamide containing plates essentially as described in WO2007060247. The 3-copy construct was amplified using PCR including 1000 bp 5′ and 3′ flanking regions (SEQ ID Nos: 59 and 60, respectively) that induce the integration in the invertase locus and a selectable marker. The amplified DNA fragment was purified and transformed to Kluyveromyces lactis strain GG799 by electroporation. Transformants were tested for beta-lactoglobulin production using MTP fermentations.

3. Production of β-Lactoglobulin A and B in K. lactis

Single transformants were inoculated on MTP agar plates with YEPhD and incubated for 2-5 days at 300° C. From the MTP agar plates cells were picked and transferred to HDWP with 200 μl YEPhD and incubated at 300° C., 750 rpm and 80% humidity for 24 hours. 24-well plates were inoculated by transferring²⁰ μl of the pre-culture to 2 ml slow release glucose medium (slow glucose release medium): Yeast Extract 10 g/l, MES hydrate 30 g/l, K2HPO4 4.5 g/l, NaH2PO4.1aq 1.5 g/l, Ammonium sulfate 7.5 g/l, Starch (Zulkowsky) 30 g/l. Addition of gluco-amylase slowly releases glucose from the starch during fermentation). Plates were incubated at 300° C., 550 rpm and 80% humidity. After 3 days, cells were centrifuged for 10 min at 2750 rpm, and 200 μl supernatant was transferred to LowBind MTP plates and analysed using LC-MS.

Food grade protein compositions were produced as described in Example 1.

### Medium Compositions

YEP2D medium: 10 g/l yeast extract, 20 g/l bacto-peptone, 40 g/l glucose. pH was set to pH 6.7 with 4N NaOH. Medium was autoclaved for 30 minutes at 110° C.

YEP2D/MES medium: 10 g/l yeast extract, 20 g/l bacto-peptone, 40 g/l glucose, 20 g/l MES. pH was set to pH 6.7 with 4N NaOH. Medium was autoclaved for 30 minutes at 110° C.

YEP2D plates contained YEP2D medium with 1.8-2% agar. Medium was autoclaved for 30 minutes at 110° C. and poured in petri dishes.

### Buffer Composition

NaOH-MES buffer: prepare MES-buffer at pH 6.05 containing 50 g/kg MES and dilute 1 volume of 4 N NaOH with 7 volumes of the MES buffer. 4. Results

Best performing strains produced about 30 mg/l of β-lactoglobulin product. Whole mass analysis by LC-MS according to the method described in Example 1 revealed that about 65% of the protein is intact and comprises the mature sequence of the protein. In the remaining fraction variation was observed at the N-terminus.

### Example 4

This example describes the analysis of the primary and secondary structure of the recombinant β-lactoglobulin protein compositions produced according to Examples 1 to 3.

### 1. Primary Structure

The identity and relative amounts the β-lactoglobulin proteins present in the compositions produced as described in Examples 1-3 was determined by LC-MS/MS follows.

Calibration lines were made with β-lactoglobulin A and B standards (Sigma L7880 and L8005, respectively) in a concentration range of 0, 5, 10, 20, 40, 80, 100 μg/mL in empty producing strains.

For sample preparation of the calibration lines and the supernatant samples the following procedure was performed: 10 μl BSA (10 g/l) was added to all samples. For protein precipitation, 210 μL of 20% TCA was added to the samples, and the solution was put 4° C. for 1 hour. After precipitation of the proteins, the samples were centrifuged for 10 min at 2250 rcf and 4° C. The supernatant was removed and the pellet is washed once with 200 μl−20° C. acetone. The washed pellet was centrifuged for 10 min at 20817 rcf and 4° C. The supernatant was removed by drying with the N2 air for 10 min.

Protein digestion was performed as follows: after drying the pellets were resolved in 50 μl 50 mM NaOH. 250 μL 100 mM NH4HCO3 was added. For reducing 10 μl 250 mM DTT was added and incubated for 30 min at 55° C. in a thermomixer at 1000 rpm. For alkylation 10 μl of 275 mM IAA was added and incubated for 30 min at room temperature in the thermomixer at 1000 rpm in the dark. Remaining IAA was quenched by placing all samples in the light for 10 min. 15 μl 0.25 μg/μl Trypsin was added and incubated over night at 37° C. in a thermomixer at 1000 rpm. The remaining digest was stored at 4° C.

The digested calibration lines and supernatant samples were analyzed on the Q Exactive plus (2) (Thermo Fisher), equipped with an Ultimate 3000 (Thermo Fisher). The chromatographic system consists of a UPLC CSH C18, 130 Å, 1.7 μm, 2.1 mm×50 mm+ACQUITY UPLC Col. In-Line Filter 0.2 μm, 2.1 mm, which is kept at 50° C. and gradient elution using A: 0.1% Formic Acid in water, and B: Formic Acid 0.1% in Acetonitrile, and a flow-rate of 400 ul/min. The gradient started at 5% B, then linear increasing to 35% B in 10 minutes, directly increasing to 80% B and kept here for 2 minutes, then directly decreasing to 5% B and kept here for 2 minutes, for re-equilibrating the LC-MS system.

The data was collected in so-called Data Independent Acquisition (DIA) LC-MS/MS. Raw data was processed using Spectronaut 1.4 matching the Uniprot extraction from Komagataella pastoris (=P. pastoris), for identification as well as quantification.

The identity and relative amounts of the proteins present in the recombinant β-lactoglobulin A and B compositions produced according to Examples 1-3 are provided in Tables 2-4, respectively.

TABLE 2
Identity and relative amounts of proteins present in the
recombinant bovine β-lactoglobulin A and B compositions
produced by Pichia pastoris according to Example 1.

| | | | |
|---|---|---|---|
| | Relative | | |
| Variant | amount (%) | Seq ID | Variant type |

B-Lac A 01

| | | | |
|---|---|---|---|
| Mature | 6 | 1 | Wild type bovine sequence |
| Mature + EAEAM | 46 | 4 | Elongation |
| Mature + EAM | 35 | 5 | Elongation |
| Mature + M | 9 | 6 | Elongation |
| Mature − L | 3 | 8 | Truncation |
| Mature − LI | 1 | 9 | Truncation |
| Mature − LIV | 1 | 10 | Truncation |

B-Lac B 01

| | | | |
|---|---|---|---|
| Mature | 4 | 2 | Wild type bovine sequence |
| Mature + EAEAM | 57 | 19 | Elongation |
| Mature + EAM | 34 | 20 | Elongation |
| Mature + M | 1 | 21 | Elongation |
| Mature − L | 3 | 23 | Truncation |
| Mature − LI | 2 | 24 | Truncation |

TABLE 3
Identity and relative amounts of proteins present in the
recombinant bovine β-lactoglobulin A and B compositions
produced by Aspergillus niger according to Example 2.

| | | | |
|---|---|---|---|
| | Relative | | |
| Variant | amount (%) | Seq ID | Variant type |

B-Lac A 02

| | | | |
|---|---|---|---|
| Mature | 2 | 1 | Wild type bovine |
| | | | sequence |
| Mature − L | 6 | 8 | Truncation |
| Mature − LIVT | 9 | 11 | Truncation |
| Mature (*pyro | 9 | 12 | Truncation |
| Q) − LIVT | | | |
| Mature − LIVTQ | 41 | 13 | Truncation |
| Mature − | 2 | 14 | Truncation |
| LIVTQT | | | |
| Mature − | 39 | 15 | Truncation |
| LIVTQTM | | | |
| Mature − | 1 | 17 | Truncation |
| LIVTQTMKG | | | |
| Mature − | 1 | 18 | Truncation |
| LIVTQTMKGL | | | |

B-Lac B 02

| | | | |
|---|---|---|---|
| Mature | 2 | 2 | Wild type bovine |
| | | | sequence |
| Mature − L | 6 | 23 | Truncation |
| Mature − LI | 9 | 24 | Truncation |
| Mature − LIV | 41 | 25 | Truncation |
| Mature − LIVT | 2 | 26 | Truncation |
| Mature − LIVTQ | 39 | 27 | Truncation |
| Mature − | 1 | 28 | Truncation |
| LIVTQT | | | |
| Mature − | 1 | 29 | Truncation |
| LIVTQTM | | | |

TABLE 4
Identity and relative amounts of proteins present in
the recombinant bovine β-lactoglobulin A and B compositions
produced by K. lactis according to Example 3.

| | | | |
|---|---|---|---|
| | Relative | | |
| Variant | amount (%) | Seq ID | Variant type |

B-Lac A 03

| | | | |
|---|---|---|---|
| Mature | 18 | 1 | Wild type bovine |
| | | | sequence |
| Mature + R | 5 | 7 | elongation |
| Mature − L | 9 | 8 | Truncation |
| Mature − LI | 5 | 9 | Truncation |
| Mature − LIV | 8 | 10 | Truncation |
| Mature − LIVT | 38 | 11 | Truncation |
| Mature (*pyro | 5 | 12 | Truncation |
| Q) − LIVT | | | |
| Mature − | 8 | 14 | Truncation |
| LIVTQT | | | |
| Mature − | 3 | 15 | Truncation |
| LIVTQTM | | | |
| Mature − | 1 | 16 | Truncation |
| LIVTQTMK | | | |

B-Lac B 03

| | | | |
|---|---|---|---|
| Mature | 34 | 2 | Wild type bovine |
| | | | sequence |
| Mature + R | 7 | 22 | Elongation |
| Mature − L | 19 | 23 | Truncation |
| Mature − LI | 7 | 24 | Truncation |
| Mature − LIV | 7 | 25 | Truncation |
| Mature − LIVT | 18 | 26 | Truncation |
| Mature − | 6 | 28 | Truncation |
| LIVTQT | | | |
| Mature − | 1 | 29 | Truncation |
| LIVTQTM | | | |

### 2. Secondary Structure

To determine secondary structure of the microbial recombinant β-lactoglobulin variant A and B compositions produced as described in Example 1, in comparison to the mature wild type variants of bovine β-lactoglobulin, circular dichroism (CD) spectroscopy was performed on samples in the spectral region (190-250 nm).

CD data was fitted using the BeStSel algorithm (Micsonai, András et al. “BeStSel: a web server for accurate protein secondary structure prediction and fold recognition from the circular dichroism spectra.” Nucleic acids research vol. 46, W1 (2018): W315-W322. doi:10.1093/nar/gky497) as a means to provide an estimate of the secondary structure composition of α-helix, β-sheet, turns, as well as other structural elements. The secondary structure composition and NRMSD (normalized root-mean-square deviation) from the algorithm fitting are presented in Table 5.

The CD spectra demonstrated that all protein samples are folded and possess secondary structure. The calculated secondary structure for wild type bovine β-lactoglobulin was similar to that reported in the literature (Brownlow et al., 1997. Structure. 5481-495. doi:10.2210/PDB1BEB/PDB; and Gutierrez-Magdaleno et al., 2013., J. Mol. Recognit. 26 67-75. doi:10.2210/PDB4GNY/PDB). The protein secondary structure estimates as determined by circular dichroism (CD) for the recombinant proteins (see Table 5) allow for comparison in respect of differences to that of wild type bovine β-lactoglobulin e.g. in the content of α-Helices, where the % ranged from 0-5.4% or between 0 and 2.5 fold the α-Helix content of wild type bovine β-lactoglobulin.

TABLE 5
Estimated secondary structure composition of bovine recombinant
β-lactoglobulin A and B samples (% of each structural element)
compared with wild type bovine β-lactoglobulin A and B.

| | Bovine | | | Bovine | | |
|---|---|---|---|---|---|---|
| | B-Lac A | | | B-Lac B | | |
| | (wild | B-Lac A | B-Lac A | (wild | B-Lac B | B-Lac B |
| | type) | 01 | 02 | type) | 01 | 02 |
| α-Helix | 2.1 | 5.4 | 3.6 | 1.5 | 0.7 | 0 |
| β-Antiparallel | 43.6 | 41.5 | 41.9 | 48.7 | 47.3 | 47.9 |
| sheet | | | | | | |
| β-Parallel | 2.5 | 0 | 2.2 | 0 | 0 | 0 |
| sheet | | | | | | |
| Turn | 11.4 | 12.4 | 12.8 | 13 | 13 | 12.7 |
| Other non- | 40.5 | 40.8 | 39.6 | 36.7 | 38.9 | 39.4 |
| defined | | | | | | |
| NRMSD: | 0.0323 | 0.0245 | 0.0269 | 0.0318 | 0.0295 | 0.0301 |

### 3. Protein Quality

Based on the amino acid sequences of the variants present and their relative proportions as described in Tables 2 and 3 above, respective protein qualities of the recombinant proteins in the compositions were calculated using a DIAAS calculator (as described by FAO (FAO. FOOD AND. NUTRITION. PAPER. 92. Dietary protein quality evaluation in human nutrition. Report of an. FAO Expert Consultation. 31 Mar.-2 Apr. 2011).

Wild type bovine β-lactoglobulin has a DIAAS score of 0.91.

B-Lac A 01 had a calculated DIAAS score of 0.89 (98% of the score for wild type bovine β-lactoglobulin). B-Lac B 02 had a calculated DIAAS score of 0.93 (102% of the score for wild type bovine β-lactoglobulin).

### Example 5

This example investigates the performance of protein compositions of the invention in an acid gel yoghurt model.

### 1. Preparation of Yoghurt Models

Samples of freeze-dried, native (wild type) β-lactoglobulin (isolated from bovine milk according to the method of Manderson et al., 1998 J Agri Food Chem 46: 5052-5061, which modified the method of Mailliart and Ribadeau-Dumas, 1988 J Food Sci 53: 743-752) or recombinant β-lactoglobulin protein compositions were dissolved in water to a minimum concentration of 25 mg/mL. The concentration of each sample was determined by UV absorbance using an extinction coefficient of 9.5 as described in Pace et al., 1995 (Protein Science, 4(11), 2411-2423). Reconstituted skim milk of 20% total solids (w/w) was prepared from low heat skim milk powder and water. Experimental milk samples with 0 to 1% β-lactoglobulin added, were prepared by adding the β-lactoglobulin and water to the 20% total solids skim milk sample to get the required level of β-lactoglobulin in a 10% total solids skim milk.

Sub-samples (6 ml) of milk with the required level of β-lactoglobulin were transferred to glass tubes (8 mL total volume) and heated, with continuous rocking at 80° C. for 30 min in an oil bath preset to the required temperature. After heating, the heated milk samples comprising 0 to 1% added β-lactoglobulin were acidified using 2% (w/w) glucono-δ-lactone (GDL) at 30° C. to form acid gels.

### 2. Gelation Time, pH and Final Stiffness

For each acid gel sample, the required level of GDL was added to the milk and the milk quickly mixed. One sub-sample was placed in a water bath at 30° C. and the pH measured at various time intervals up to 3 hours. The rheological properties of a second sub-sample during acidification was determined using a TA AR2000 rheometer (TA Instruments UK, Cirencester, Gloucestershire, England) using a cone (4 cm, 4°) and plate geometry. The milk with GDL (˜1.2 mL) was placed on the rheometer plate and the cone lowered into position. Evaporation during measurements was minimised by using a water trap/cover arrangement over the sample. An oscillation test was used with the frequency set to 0.1 Hz, the strain was less than 0.5% and the temperature was maintained at 30° C. A measurement of the stiffness was made every five minutes for a total of 3 hours. The gelation time, gelation pH and final stiffness after 3 hours were recorded and the results are presented in Table 6.

TABLE 6
Acid gelation properties of milk with different levels of added wild type
β-lactoglobulin or fermentation derived β-lactoglobulin.

| | | | |
|---|---|---|---|
| | gelation | gelation | final |
| Sample | time (min) | pH | stiffness (Pa) |

Wild type β-lactoglobulin A

| | | | |
|---|---|---|---|
| milk | 26.5 | 5.35 | 268.3 |
| milk + 0.25% wild | 21.1 | 5.46 | 334.1 |
| type β-lactoglobulin A | | | |
| milk + 0.5% wild | 19.6 | 5.49 | 378.7 |
| type β-lactoglobulin A | | | |
| milk + 0.75% wild | 18.4 | 5.51 | 383.3 |
| type β-lactoglobulin A | | | |
| milk + 1.0% wild | 16.5 | 5.55 | 413.7 |
| type β-lactoglobulin A | | | |

B-Lac B 02 (A. niger)

| | | | |
|---|---|---|---|
| milk | 26.1 | 5.36 | 252 |
| milk + 0.25% B-Lac B 02 | 20.7 | 5.47 | 251.5 |
| milk + 0.5% B-Lac B 02 | 18.8 | 5.50 | 250.5 |
| milk + 0.75% B-Lac B 02 | 18.4 | 5.51 | 264.5 |
| milk + 1.0% B-Lac B 02 | 16.9 | 5.54 | 298.3 |

B-Lac B 03 (K. lactis)

| | | | |
|---|---|---|---|
| milk | 26.0 | | |
| milk + 0.25% B-Lac B 03 | 21.1 | 5.46 | 287.8 |
| milk + 0.5% B-Lac B 03 | 19.5 | 5.49 | 329.6 |
| milk + 0.75% B-Lac B 03 | 17.2 | 5.53 | 382.5 |
| milk + 1.0% B-Lac B 03 | 16.0 | 5.56 | 444.6 |

B-Lac A 01 (P. pastoris)

| | | | |
|---|---|---|---|
| milk | 26.5 | 5.35 | 268.3 |
| milk + 0.25% B-Lac A 01 | 21.1 | 5.46 | 279.5 |
| milk + 0.5% B-Lac A 01 | 21.9 | 5.44 | 268.9 |
| milk + 0.75% B-Lac A 01 | 21.5 | 5.45 | 239.1 |
| milk + 1.0% B-Lac A 01 | 19.6 | 5.49 | 282.5 |

### Example 6

This example investigates the properties of protein bars prepared using a protein composition of the invention.

### 1. Preparation of Protein Bars

Model protein dough bars (cold pressed) were prepared according to the formulation and method of Imtiaz et al., 2012 (Journal of Texture Studies, 43, 275-286). The nutritional composition was standardised to 30% protein, ˜50% carbohydrate and 9% fat.

The composition of the formulations is shown in Table 7.

Protein powders used were:
Whey protein isolate—NZMP SureProtein™ WPI895 (˜90% protein, ˜69 g βlg/100 g powder),
B-Lac A 01 (˜65% protein),
Pea protein isolate Empro E86 from Emsland Stärke, Germany (˜87% protein dry basis; 82.6% protein as is).

Blends of protein consisted of 90% pea protein isolate and 10% of either WPI895 or B-Lac A 01.

The amounts of glucose syrup, glycerine and lecithin were fixed in all formulations. Glucose syrup and glycerine function to lower water activity and enable an ambient—stable product. Glycerine also acts as a plasticiser.

TABLE 7
Protein bar formulations.

| | | % (w/w) | % (w/w) | % (w/w) |
|---|---|---|---|---|
| | % (w/w) | 100% pea | 90 | 90 pea: 10 |
| Ingredient | 100% WPI | protein | pea: 10WP | B-Lac A 01 |
| WPI895 | 33.33 | — | 3.33 | — |
| Pea protein | — | 36.32 | 32.69 | 32.69 |
| B-Lac A 01 | — | — | | 3.33 |
| Glucose syrup | 34.9 | 34.9 | 34.9 | 34.9 |
| Glycerine | 17.4 | 17.4 | 17.4 | 17.4 |
| Maltodextrin | 5.37 | 3.38 | 3.35 | 2.06 |
| DE20 | | | | |
| Hydrogenated | 8.5 | 7.5 | 7.83 | 7.83 |
| coconut fat | | | | |
| Lecithin | 0.5 | 0.5 | 0.5 | 0.5 |
| TOTAL | 100 | 100 | 100 | 100 |

The maltodextrin and protein ingredient(s) were weighed into a Hobart mixing bowl (Model N-50). The glucose syrup and glycerine were heated to 50° C. and in another container the fat and lecithin heated until melted. The heated syrups and melted fats were added to the dry ingredients in the mixing bowl and then mixed at Speed 1 for 90 s and then the bowl was scraped down. Mixing (Speed 2) was continued until a homogeneous mass was obtained. The mass was “poured” into a frame (˜16 mm deep) and spread evenly and pressed down. The protein doughs were left overnight and then cut into bars and packaged into foil laminate sachets and heat sealed. They were stored at ambient (˜20° C.) and at −18° C. (reference samples).

### Response Variables

The ease of forming the doughs and any cold flow were noted. Over time any colour changes were monitored and the peak firmness of the bars measured (TA-XT2 Texture Analyser, 5 mm cylindrical probe at 1 mm/s to a depth of 8 mm and withdrawn at 10 mm/s as per Imtiaz et al. (2012)).

### 2. Results

All bar formulations were able to be processed in a similar manner and there was no evidence of cold flow, where the bar sags under its own weight after cutting.

The firmness of the bars over storage time is shown in Table 8.

TABLE 8
Protein Bar Peak Firmness Results as a Function of Storage Time

| | Peak | Peak Force | Peak Force | Peak Force |
|---|---|---|---|---|
| 30% protein | Force (g) | (g) at 1 | (g) at 4 | (g) at 7 |
| model dough | at time | month | months | months |
| bars containing | zero | ambient | ambient | ambient |
| WPI895 | 2533 ± 273 | 2979 ± 460 | 4393 ± 371 | 3385 ± 285 |
| Pea Protein | 1931 ± 243 | 2409 ± 543 | 2548 ± 513 | 3611 ± 299 |
| 90 Pea: 10 WPI895 | 1575 ± 79 | 1657 ± 155 | 2112 ± 167 | 3117 ± 256 |
| 90 Pea: 10 B-Lac A 01 | 1091 ± 230 | 1696 ± 277 | 2311 ± 345 | 2887 ± 107 |

### Example 7

This example investigates the foaming properties of compositions of the invention comprising recombinant β-lactoglobulin proteins.

WPI895 is reconstituted with reverse osmosis water as a 10% protein solution and left to hydrate fully for >2 hours. The resulting solution has a pH of 6.7-7. A 110 mL sample is whipped using a Hobart mixer (Model N-50) and a balloon whisk, speed 3. At 5, 10- and 15-minutes, whipping is stopped and overrun measured.

$$Overrun ( \% ) = ( {V}_{f} - {V}_{i} ) / {V}_{i} \times 100$$

Where Vᵢ=initial volume (110 mL) and V_{f}=final volume

Protein compositions comprising β-lactoglobulin are prepared according to Example 1. Samples are prepared and whipped as described above.

After 15 minutes whipping, a subsample of the foam is transferred to a 250 mL powder funnel (with fabric gauze covering the neck of the funnel) and left to stand at ambient for 60 minutes. The time for the first drop of liquid to leave the funnel is recorded. The volume of liquid passing through the funnel (serum volume) is recorded over this time. The results are shown in Table 9.

TABLE 9
Foam overrun and foam stability of WPI895 and that of recombinant
B-Lactoglobulin A and B variants produced by Pichia pastoris.

| | Overrun (%) | | | Time to first | Serum volume (mL) | | | | |
|---|---|---|---|---|---|---|---|---|---|
| | 5 | 10 | 15 | serum leakage | 10 | 15 | 30 | 45 | 60 |
| Sample | min | min | min | (m · s) | min | min | min | min | min |
| WPI895 | 400 | 630 | 1040 | 13.17 | 0 | <1 | 8 | 10 | 11 |
| B-Lac A 01 | | | | | | | | | |
| B-Lac B 01 | | | | | | | | | |

A high overrun indicates a high volume foam and a longer time for initial foam breakdown and low values for serum volume after 60 minutes indicates a stable foam.

The results show that β-lactoglobulin compositions of the invention exhibit similar or enhanced foaming properties to the WPI895 sample.

### Example 8

This example describes the production of Pichia pastoris strains having a pep4 knockout (Δpep4) expressing a plurality of recombinant β-lactoglobulin proteins, and purification of the proteins to produce compositions of the invention.

1. Plasmid pCASPP-05 Containing CAS9 and Guide RNA Targeting PEP4

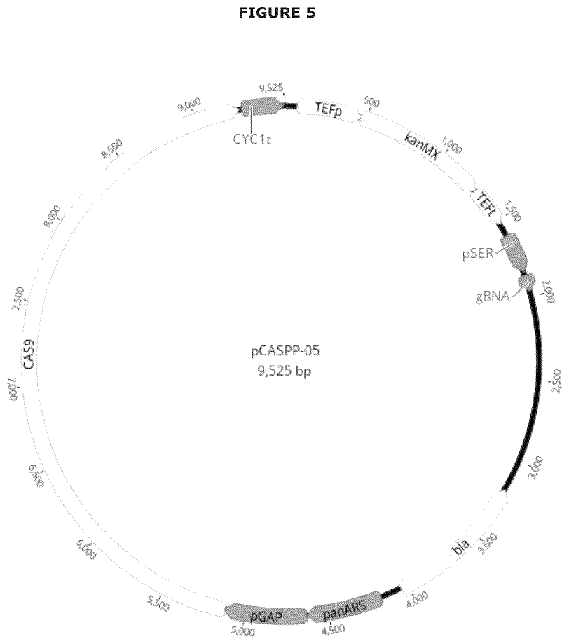

The functional knockout of the pep4 gene was accomplished using CRISPR-CAS9 to support integration of a fragment encoding bovine β-lactoglobulin B and deletion of the PEP4 gene sequence. An “all in one” plasmid named pCASPP-05 (shown in FIG. 5) was used, which expressed both CAS9 and the guide RNA (gRNA) targeting the pep4 sequence. The construction of pCASPP-05 (SEQ ID 64) was done using standard Gibson cloning methods. The plasmid contains the CAS9 gene expressed with the pGAP promoter and CYC1t terminator (SEQ ID 65) and the pep4 gRNA expressed with the pSER promoter (SEQ ID 66). The plasmid contains the kanMX selectable marker and an autonomously replicating sequence to select and maintain the plasmid in the cell after transformation to Pichia.

### 2. Preparing the β-Lactoglobulin B DNA Pep4 Integration Fragments

The gene coding for wild type mature bovine β-lactoglobulin B was ordered at synthetic DNA provider ATUM (CA, USA). The β-lactoglobulin B encoding sequences were fused behind the α-mating factor from S. cerevisiae followed by a Kex2 processing site composed of lysine, arginine (KR) at the start of the β-lactoglobulin B amino acid sequence. The β-lactoglobulin B open reading frame was placed under control of the methanol inducible AOX1 promoter and the AOX1 terminator was placed at the end of the ORF forming the β-lactoglobulin B expression cassette (SEQ ID No: 67). The integration fragment transformed to Pichia was obtained by PCR amplification. The β-lactoglobulin B expression cassette (SEQ ID No: 67) was used as DNA template in the PCR. Forward primer DBC-26963 (SEQ ID No: 68) and reverse primer DBC-26964 (SEQ ID No: 69) were used to amplify the β-lactoglobulin B expression cassette attaching 60 bp flanking regions to facilitate homologous recombination in the genome and deleting the pep4 DNA sequence. The pep4 deletion fragment is listed as SEQ ID No: 70. The PCR used Q5® High-Fidelity DNA Polymerase from NEW ENGLAND Biolabs and the protocol used is described in Tables 10 and 11. After PCR amplification, DNA was purified using the commercial PCR purification kit “DNA Clean & Concentrator Kits” from Zymo research. 3. Preparing the β-lactoglobulin A DNA pep4 integration fragments

The β-lactoglobulin A DNA pep4 integration fragments were prepared as described for β-lactoglobulin B, except that a KREAEAM processing site was used instead.

TABLE 10
PCR protocol to amplify integration fragment

| | Volume | | |
|---|---|---|---|
| | (μl) | Final concentration | |
| 5X Q5 Reaction Buffer | 10 | 1X | |
| 10 mM dNTPs | 1 | 200 | μM |
| 10 μM Forward Primer | 2.5 | 0.5 | μM |
| 10 μM Reverse Primer | 2.5 | 0.5 | μM |
| Template DNA | 1 | 1 | ng |
| Q5 High-Fidelity DNA Polymerase | 0.5 | 0.02 | U/μl |

| | |
|---|---|
| Nuclease-Free Water | 32.5 |

TABLE 11
PCR protocol used to amplify integration fragment

| Temp (° C.) | Duration | |
|---|---|---|
| 98 | 30 | s |
| 98 | 10 | s |
| 55 | 30 | s |
| 72 | 2 | min |
| Repeat previous 3 steps 5 times | | |
| 98 | 10 | s |
| 65 | 30 | s |
| 72 | 2 | min |
| Repeat previous 3 steps 35 times | | |
| 72C | 2 | min |

### 4. Transformation of the β-Lactoglobulin A and B DNA Pep4 Integration Fragments

The vector pCASPP-05 and the purified PCR fragment containing the β-lactoglobulin A or B expression cassette flanked with 50 bp integration flanks were transformed to the Pichia pastoris strain NRRL-Y11430 (a wild type strain received from the ARS Culture Collection). The amount of DNA used in the transformation for both plasmid and the integration fragment was 1 μg. Transformation procedure was performed according to condensed electroporation protocol using freshly prepared solutions (Lin-Cereghino et al. Biotechniques (2005) 38, (1):44-48). Transformants were plated on G418 Selective YEPDS agar (YPDS: 1% yeast extract, 2% peptone, 2% glucose, 1M sorbitol, 2% agar) with G418 added to a final concentration of 750 μg/ml. Plates were incubated at 30° C. for 72 h. Single colonies were picked from the plates and transferred to 96-well MTP agar plates containing YEPHD agar YEPhD-agar (BBL Phytone peptone 20.0 g/l, Yeast Extract 10.0 g/l, Sodium Chloride 5.0 g/l, Agar 15.0 g/l and 2% glucose) and 500 μg/ml G418. MTP agar plates were incubated for 72 hours at 30° C. From the MTP agar plate transformants were inoculated into 200 μl YEPHD medium (BBL Phytone peptone 20.0 g/l, Yeast Extract 10.0 g/l, Sodium Chloride 5.0 g/l and 2% glucose) in half deepwell plates (HDWP) using a pin and incubated overnight in an INFORS MTP incubator at 300° C., 750 rpm and 80% humidity. This was used as pre-culture for the protein production experiment.

5. Production of Recombinant β-Lactoglobulin A and B in P. pastoris Δ Pep4 Host Cells

Recombinant β-lactoglobulin was produced in P. pastoris Δpep4 according to the method described above in Example 1.

Food grade protein compositions were produced using a 10 litre scale fermenter, followed by downstream processing including removal of biomass by centrifugation, ultrafiltration for concentration and dialysis, anionic exchange for purification, ultrafiltration for concentration and dialysis for removal of salts and drying by freeze drying.

### 6. Primary Structure

The identity and relative amounts of the β-lactoglobulin proteins present in the compositions was determined by LC-MS/MS follows as described above in Example 4. The results are presented in Table 12.

TABLE 12
Identity and relative amounts of proteins present in the
recombinant bovine β-lactoglobulin A and B compositions
produced in a Δpep4 Pichia pastoris host cell.

| | Relative amount (%) | | | | |
|---|---|---|---|---|---|
| Variant | Rep 1 | Rep 2 | Rep 3 | Seq ID | Variant type |

Δpep4 B-Lac A

| | | | | | |
|---|---|---|---|---|---|
| Mature | 8 | 7 | 7 | 1 | Wild type bovine |
| | | | | | sequence |
| Mature + | 51 | 53 | 51 | 4 | Elongation |
| EAEAM | | | | | |
| Mature + EAM | 28 | 27 | 28 | 5 | Elongation |
| Mature + M | 5 | 5 | 5 | 6 | Elongation |
| Mature − L | 4 | 4 | 5 | 8 | Truncation |
| Mature − LI | 2 | 2 | 2 | 9 | Truncation |
| Mature − LIV | 2 | 2 | 2 | 10 | Truncation |
| Mature − LIVT | 1 | 1 | 1 | 11 | Truncation |

Δpep4 B-Lac B

| | | | | | |
|---|---|---|---|---|---|
| Mature | 16 | 15 | 15 | 2 | Wild type bovine |
| | | | | | sequence |
| Mature − L | 6 | 5 | 6 | 23 | Truncation |
| Mature − LI | 4 | 3 | 4 | 24 | Truncation |
| Mature − LIV | 12 | 12 | 11 | 25 | Truncation |
| Mature − LIVT | 25 | 26 | 26 | 26 | Truncation |
| Mature (*pyro | 35 | 37 | 36 | 61 | Truncation |
| Q) − LIVT | | | | | |
| Mature − | 2 | 2 | 2 | 28 | Truncation |
| LIVTQT | | | | | |

### Example 9

This example describes methods for detecting aspartyl protease activity in recombinant milk protein compositions via detection of κ-casein degradation.

### 1. Methods

Skim milk (20% TS) was mixed with a recombinant protein composition produced as described in Examples 1 or 8 above or with standard, milk-derived β-lactoglobulin and water to provide a skim milk content of 10% TS and a β-lactoglobulin content of 0, 0.25, 0.5, 0.75 or 1% w/w.

The milk samples were held for one hour to equilibrate. Sub-samples of each mixture were heated at 80° C. for 30 min or 120° C. for 10 minutes. The unheated and heated samples were held at 5° C. for 6 hours and then analyzed by traditional sodium dodecyl sulphate polyacrylamide gel electrophoresis (SDS PAGE) or “lab-on-a-chip” microfluidic SDS electrophoresis. Detailed methods for “lab-on-a-chip” is provided in S. G. Anema 2009, International Milk Journal 19 (2009) 198-204”.

### 2. Results—SDS PAGE

For the unheated samples with bovine milk-derived b-lactoglobulin, the gel showed no change in the intensity of the k-casein band and no appearance of a peptide peak in the region expected for para-k-casein. In contrast, the unheated samples comprising recombinant b-lactoglobulin produced in Pichia cells with the pep4 gene according to Example 1, the band intensity of the k-casein band decreased and a peptide band consistent with that of para-k-casein appeared. This effect was more pronounced as the level of b-lactoglobulin increased from 0.25 to 1.0%.

When bovine milk-derived b-lactoglobulin was heated, the gel also showed no change in the intensity of the k-casein band and no appearance of a peptide peak in the region expected for para-k-casein. In contrast, in the heated samples with the recombinant b-lactoglobulin produced in cells with the pep4 gene according to Example 1, the band intensity of the k-casein band decreased and a peptide band consistent with that of para-k-casein appeared. The conversion was more pronounced as the level of b-lactoglobulin increased from 0.25 to 1.0%, but was less pronounced than the unheated samples at each level of added recombinant b-lactoglobulin. The loss of the k-casein band and the appearance of a para-k-casein band were therefore clear indicators of aspartyl protease activity in the fermentation derived b-lactoglobulin preparation.

Unheated and heated subsamples with 0.5% fermentation derived b-lactoglobulin and an unheated sample with 0.5% milk derived b-lactoglobulin were held for 24 hours at 5° C. and then analyzed by traditional or chip-based sodium dodecyl sulphate polyacrylamide gel electrophoresis. For both the unheated and heated samples with bovine milk derived b-lactoglobulin, the k-casein peak was of normal intensity and there was no peptide peak in the region expected for para-k-casein. For the samples with b-lactoglobulin produced in cells with the pep4 gene, the k-casein peak in both the heated and unheated samples had markedly reduced in size and a peak in the position expected for para-k-casein was present. The loss of k-casein and the para-k-casein peak was more pronounced for the unheated sample than the heated sample. The reduction of the k-casein peak intensity and the appearance of a para-k-casein peak were therefore clear indicators of aspartyl protease activity in the fermentation derived b-lactoglobulin preparations.

### 3. Results—Lab on a Chip

Results obtained for the following proteins held for 24 hours at 5° C. is presented in Table 13.
B-lac A 01 (produced in a host cell with the pep4gene according to Example 1).
B-lac B 01 (produced in a host cell with the pep4gene according to Example 1).
Δpep4 B-lac A (produced in a Δpep4 host cell according to Example 8)
Δpep4 B-lac B (produced in a Δpep4 host cell according to Example 8).

B-lac A 01 was also heated at 80° C. and 120° C. for 30 and 10 minutes, respectively, then held at 5° C. for 24 hours. After the 80° C. treatment, the peak area was 249.48 and 0 for κ-casein and para-κ-casein, respectively. After 24 hours, the peak area was 115.2 and 35.82 for κ-casein and para-κ-casein, respectively, indicating that κ-casein had degraded by 54%. After the 120° C. treatment, the peak area was 249.6 and 0 for κ-casein and para-κ-casein, respectively. After 24 hours, the peak area was 179.4 and 16.12 for κ-casein and para-κ-casein, respectively, indicating that κ-casein had degraded by 72%.

TABLE 13
Lab on a chip results

Peak area

| Sample | Protein | 0 h | 24 h |
|---|---|---|---|
| B-lac A 01 | κ-casein | 255 | 91.5 |
| | | | (64% degraded) |
| | para-κ-casein | 0 | 146.85 |
| | formed | | |
| B-lac A 01 | κ-casein | 253.98 | 138.72 |
| | | | (46% degraded) |
| | para-κ-casein | 0 | 56.1 |
| | formed | | |
| Δpep4 B-lac | κ-casein | 260.9 | 262.1 |
| B | | | |
| | para-κ-casein | 0 | 0 |
| | formed | | |
| Δpep4 B-lac | κ-casein | 251.2 | 251.8 |
| A | | | |
| | para-κ-casein | 0 | 0 |
| | formed | | |

### Example 10

This example investigates the heat stability of recombinant milk protein compositions of the invention.

### Unheated Beverage Storage Stability and Heat Stability

Skim milk (10% w/w total solids) comprising 1% w/w of recombinant β-lactoglobulin prepared. Sodium azide (0.02% w/v) was added as a preservative.

The recombinant β-lactoglobulin was provided by the following compositions prepared according to Examples 1 and 8.
B-lac A 01
B-lac B 01
Δpep4 B-lac A
Δpep4 B-lac B

Samples of each milk (˜1 mL) were placed in sealed glass bottles (Wheaton brand bottles with an 8 mL total volume) and held for different times (0.15, 4.5 or 16 hours) at ambient temperature (˜22° C.). After holding for the required times, the samples were tested for heat stability by placing the vials horizontally in a rack and then in an oil bath preset to 140° C. The samples were rocked at ˜10 cycles per minute which moved the milk from one end of the vial to the other. The samples were monitored until they visibly coagulated. The time at which they coagulated was recorded as the heat coagulation time. The results are provided in Table 14.

TABLE 14
Sample description, enzyme status and heat coagulation
times (HCT in minutes (m) and seconds (s)).

| | | Concentration of | | | |
|---|---|---|---|---|---|
| | Genetic | sample in skim | HCT at time | HCT at time | HCT at time |
| Sample | variant | milk | 0.15 h | 4.5 h | 16 h |
| B-lac A 01 | A | 1% w/w | 12 m 53 s | 00 m 51 s | 00 m 01 s |
| B-lac B 01 | B | 1% w/w | 13 m 53 s | 12 m 27 s | 10 m 31 s |
| Δpep4 B-lac A | A | 1% w/w | 19 m 54 s | 19 m 51 s | 19 m 01 s |
| Δpep4 B-lac B | B | 1% w/w | 16 m 50 s | 16 m 44 s | 16 m 05 s |

### Example 11

This example investigates the storage stability and gelation of beverages comprising recombinant milk protein compositions of the invention.

### Unheated Beverage Storage Stability and Rennet-Like Gelation

Skim milk (10% w/w total solids) with 0.5 or 1% w/w fermentation-derived β-lactoglobulin added (with active enzymes or without active enzymes) was prepared. Sodium azide (0.02% w/v) was added as a preservative. A sample of each milk (˜1.4 mL) was placed on a rheometer plate set at 20° C. A 4 cm cone was lowered into position. Light mineral oil was placed around the perimeter of the sample to prevent drying. In addition, the water trap on the cone was filled with water and the water trap cover plates were placed over the sample. The rheometer was set to increase the temperature to 40° C. within 1 minute and monitor the rheological properties for up to 12 hours at 40° C. The samples were oscillated at a frequency of 0.1 Hz and with a strain of 0.025%. The rheological properties were monitored every 20 seconds for the duration of the run. The gelation point was considered the time when the G′ increased from the base line. Table 15 shows the gelation times of the samples tested.

TABLE 15
Sample description, enzyme status and gelation time.

| | | Concentration | Aspartyl | | |
|---|---|---|---|---|---|
| | Genetic | of sample in | protease | Gelation | |
| Sample | variant | skim milk | present | time | |
| B-lac A 01 | A | 0.5% w/w | yes | 220 | min |
| B-lac A 01 | A | 1% w/w | yes | 110 | min |
| Δpep4 B-lac A | A | 0.5% | no | did not gel | |
| pep4 B-lac A | A | 1% | no | did not gel | |
| B-lac B | B | 1% | yes | 380 | minutes |

| | | | | |
|---|---|---|---|---|
| Δpep4 B-lac B | B | 1% | no | did not gel |

### Example 12

This example describes the production of Pichia pastoris strains using a KREAEA processing site (instead of KREAEAM processing site used in example 1) to express recombinant β-lactoglobulin proteins, and purification of the proteins to produce compositions of the invention. The protein compositions described in this Example were used in Examples 13 to 17.

### 1. Production of β-Lactoglobulin A Compositions PP29-30 and 006B, and (β-Lactoglobulin B Composition PP26

Strains producing β-lactoglobulin A compositions “PP29-30” and “006B”, and β-lactoglobulin B composition “PP26” with an alternative N-terminal amino-acid sequence were produced as described in example 1 in Pichia pastoris. For PP29-30 and 006B, the β-lactoglobulin A variant with SEQ ID No: 75 was used. For PP26, the β-lactoglobulin B variant with SEQ ID No: 76 was used. In SEQ ID Nos: 75 and 76 a KREAEA processing site was used in the expressed protein sequences for production instead of the KREAEAM processing site used in Example 1 sequences. As in Example 1, the β-lactoglobulin sequences were placed in a circular plasmid under the control of the AOX1 promoter and AOX1 terminator leading to the circular plasmids with expression constructs SEQ ID Nos: 77 and 78. The expression constructs were integrated in the Pichia pastoris host as described in Example 1 to produce the 3 mentioned β-lactoglobulin A and B compositions used in the examples 13 to 17.

### 2. Production of β-Lactoglobulin A Composition 007A

The β-lactoglobulin A composition 007A was produced using Pichia pastoris using a KREAEAM processing site as described in Example 1.

### 3. Production of β-Lactoglobulin A Composition 009D

The β-lactoglobulin A composition 009D was produced using Pichia pastoris as described in WO2016/029193 Example 6. Briefly, the gene coding for wild type mature bovine β-lactoglobulin A was cloned into an expression vector pLH37 fused behind the signal sequence of the α-mating factor. The gene was under the control of the methanol inducible AOX1 promoter.

### 4. Primary Structure

The identity and relative amounts of the β-lactoglobulin proteins present in the compositions were determined by LC-MS/MS as described above in Example 4. The results are presented in Table 16.

TABLE 16
Identity and relative amounts of proteins present in
the recombinant bovine β-lactoglobulin A and B compositions
produced in Pichia pastoris host cell.

| | | | |
|---|---|---|---|
| | Relative | | |
| Variant | amount (%) | Seq ID | Variant type |

β-lactoglobulin A PP29-30

| | | | |
|---|---|---|---|
| Mature | 1.0 | 1 | Wild type bovine sequence |
| Mature + EAEA | 91 | 71 | Elongation |
| Mature + EA | 7 | 72 | Elongation |
| Mature − L | 0.9 | 8 | Truncation |

β-lactoglobulin A 007A

| | | | |
|---|---|---|---|
| Mature | 10 | 1 | Wild type bovine sequence |
| Mature + | 42 | 4 | Elongation |
| EAEAM | | | |
| Mature + EAM | 28 | 5 | Elongation |
| Mature + M | 8 | 6 | Elongation |
| Mature − L | 8 | 8 | Truncation |
| Mature − LI | 1 | 9 | Truncation |
| Mature − LIV | 1 | 10 | Truncation |
| Mature − LIVT | 1 | 11 | Truncation |

β-lactoglobulin A 006B

| | | | |
|---|---|---|---|
| Mature | 28 | 1 | Wild type bovine sequence |
| Mature + EAEA | 58 | 71 | Elongation |
| Mature + EA | 10 | 72 | Elongation |
| Mature − L | 3 | 8 | Truncation |
| Mature − LIVT | 1 | 11 | Truncation |
| Mature (*pyro | 1 | 12 | Truncation |
| Q) − LIVT | | | |

β-lactoglobulin A 009D

| | | | |
|---|---|---|---|
| Mature | 35 | 1 | Wild type bovine sequence |
| Mature − L | 17 | 8 | Truncation |
| Mature − LI | 10 | 9 | Truncation |
| Mature − LIV | 15 | 10 | Truncation |
| Mature − LIVT | 15 | 11 | Truncation |
| Mature (*pyro | 7 | 12 | Truncation |
| Q) − LIVT | | | |

β-lactoglobulin B PP26

| | | | |
|---|---|---|---|
| Mature | 1.2 | 2 | Wild type bovine sequence |
| Mature + EAEA | 86 | 73 | Elongation |
| Mature + EA | 11 | 74 | Elongation |
| Mature − L | 1.6 | 23 | Truncation |
| Mature − LIVT | 0.1 | 26 | Truncation |
| Mature (*pyro | 0.1 | 61 | Truncation |
| Q) − LIVT | | | |
| Mature − LIVTQ | 0.1 | 27 | Truncation |

### Example 13

This example investigates the foaming properties of compositions of the invention comprising recombinant β-lactoglobulin proteins.

### 1. Preparation of Foams

The method was based on that of Caessens et al. (1997). J. Ag. Fd Chem., 45, 2935-2941, where a 2.5% protein solution was whipped and the volume of foam produced and its breakdown over time was measured.

Protein compositions comprising β-lactoglobulin were prepared according to Example 12. WPI895 and the β-lactoglobulin samples were reconstituted with reverse osmosis water, hydrated for at least 60 minutes, pH adjusted to 7.0, and diluted with reverse osmosis water to give a final protein content of 2.5%.

50 mL samples were whipped with a hand-held, battery operated, milk frother (circular impeller 36 mm diameter) in a graduated glass cylinder for 70 seconds. Foam and liquid volumes were monitored for 45 minutes. The measurements were performed in at least duplicate.

### 2. Results

The results are shown in Tables 17 and 18. A high foam volume indicates good air incorporation, and a slow loss in foam volume or slow liquid volume increase, indicates a stable foam.

TABLE 17
Foam volume of WPI895 and that of recombinant β-lactoglobulin A
and B variants produced by Pichia pastoris as a function of time.

Foam Volume (mL)

| | | βlgA | βlgA | βlgA | βlgB |
|---|---|---|---|---|---|
| Time (min) | WPI895 | PP29-30 | 007A | 006B | PP26 |
| 2 | 84 | 84 | 85 | 90 | 78 |
| 5 | 75 | 76.5 | 77 | 80 | 71 |
| 10 | 70 | 71.5 | 72.5 | 77 | 65 |
| 15 | 68 | 69.5 | 67.5 | 73 | 63 |
| 20 | 66 | 68.5 | 63.5 | 71 | 61 |
| 25 | 63 | 67 | 61.5 | 68 | 59 |
| 30 | 57 | 66 | 60 | 67 | 57 |
| 35 | 49 | 65 | 58.3 | 67 | 56 |
| 40 | 41 | 64 | 55.5 | 65 | 56 |
| 45 | 36 | 63 | 53.5 | 64 | 56 |

TABLE 18
Liquid volume of WPI895 and that of recombinant β-lactoglobulin
A and B variants produced by Pichia pastoris as a function of time.

Liquid Volume (mL)

| | | βlgA | βlgA | βlgA | βlgB |
|---|---|---|---|---|---|
| Time (min) | WPI895 | PP29-30 | 007A | 006B | PP26 |
| 2 | 29 | 31 | 30 | 30 | 33 |
| 5 | 37 | 37 | 36.5 | 38 | 39 |
| 10 | 41 | 39 | 40 | 41 | 41 |
| 15 | 42 | 41 | 42.5 | 43 | 43 |
| 20 | 44 | 42 | 44 | 44 | 43 |
| 25 | 45 | 43 | 45 | 44 | 45 |
| 30 | 45 | 44 | 45 | 45 | 45 |
| 35 | 45 | 44 | 45.7 | 45 | 46 |
| 40 | 46 | 44 | 46 | 46 | 46 |
| 45 | 46 | 45 | 46 | 46 | 46 |

### Example 14

This example investigates the emulsification properties of the invention comprising recombinant β-lactoglobulin proteins.

### 1. Preparation of Emulsions

The method was based on Guo & Xiong's electrical conductivity method to determine emulsifying capacity of proteins (J. Food Science, 86, 4914-4921, 2021). The electrical conductivity of emulsions prepared with different oil:protein ratios was measured. The point at which conductivity reaches zero is regarded as where phase inversion occurs from an oil-in-water emulsion to a water-in-oil emulsion.

Protein compositions comprising β-lactoglobulin were prepared according to Example 12. WPI895 was reconstituted with reverse osmosis water, stirred for at least 60 minutes, pH adjusted to 7.0, and diluted with reverse osmosis water to give a final protein content of 0.5% w/w.

To the protein solution, six aliquots of canola oil at 20, 50, 100, 200, 400, and 600 mL oil/g protein were added. The mixtures were homogenised for 3 minutes at 17,600 rpm using an Ultraturrax (IKA T25 digital disperser, Head: S25N-10G). The electrical conductivity of the emulsions was measured immediately with a conductivity meter (Wissenschaftlich-Technische Werkstätten, Model Cond 315i). Electrical conductivity data (μS/cm) were plotted against the final protein concentrations based on the initial protein concentration (c₁=5 mg/mL) and the amount of oil added. A regression line was used to determine the final protein concentration when conductivity reached zero (c₂). Emulsifying capacity was calculated according to the following formula:

$$Emulsifying capacity ( mL oil / g protein ) = ( {c}_{1} - {c}_{2} ) / ( {c}_{1} \times {c}_{2} ) \times 1000$$

### 2. Results

The electrical conductivity was measured, and data analysed as described above. Experiments were run in duplicate. The emulsifying capacity of the proteins is shown in Table 19.

TABLE 19
Emulsifying capacity of 0.5% protein solutions
of WPI895 and recombinant β-lactoglobulin
A and B variants produced by Pichia pastoris.

| Protein | Emulsifying Capacity (mL oil/g protein) |
|---|---|
| WPI895 | 1151 ± 107 |
| βlgA (PP29-30) | 1323 ± 331 |
| βlgA (007A) | 1395 ± 97 |
| βlgA (006B) | 1087 ± 74 |
| βlgB (PP26) | 1473 ± 283 |

### Example 15

This example investigates the performance of protein compositions of the invention in the preparation of heat-set gels.

### 1. Preparation of Heat-Set Gels

Samples of freeze-dried wild type β-lactoglobulin (isolated from bovine milk as described in example 5) or recombinant β-lactoglobulin protein compositions were dissolved in water to provide solutions of about 15% w/w protein. The recombinant β-lactoglobulin protein compositions were as described in Example 12. The solutions were stirred until completely dissolved and then equilibrated for at least 6 hours. The pH of each solution was adjusted to pH 7.0, and the concentration of protein was determined using UV spectrometry at 280 nm and the extinction coefficient of 0.95 cm²/g.

The β-lactoglobulin solutions were combined with water at appropriate ratios to provide 25 mL samples with β-lactoglobulin concentrations of 5.0, 6.25, 7.5, 8.75 and 10% w/w. To each solution was added 150 μL of a 25% (w/v) sodium chloride solution.

The rheological properties of samples of each sample was determined using a TA AR2000 rheometer (TA Instruments UK, Cirencester, Gloucestershire, England) using a cone (6 cm, 4°) and plate geometry. A 4 mL sub-sample of each solution was placed on the rheometer. The cone was lowered into position and oil was placed around the perimeter of the sample to minimize evaporation of the sample. Small strain rheological tests were performed using a frequency of 0.1 Hz and a strain of 0.25%. The stiffness (G′) measured at the end of steps 3 and 5.
Step 1: a constant temperature of 20° C. for 30 min.
Step 2: the temperature was increased from 20° C. to 90° C. at a rate of 5° C./min.
Step 3: a constant temperature of 90° C. for 30 min.
Step 4: the temperature was decreased from 90° C. to 20° C. at a rate of 5° C./min.
Step 5: a constant temperature of 20° C. for 30 min.

The stiffness (G′) at the end of test 3 (90° C. for 30 min) was recorded as the stiffness at 90° C., and the stiffness (G′) at the end of test 5 (20° C. for 30 min after the temperature increase) was recorded as the stiffness at 20° C. The appearance of the gel was also recorded as either transparent or opaque at the end of Step 5.

### 2. Results

The results are presented in Table 20. For all samples except 009D, tests at concentrations of 5%, 7.5%, and 10% were performed in duplicate with very similar results. Tests at concentrations of 6.25% and 8.75% were performed once. For sample 009D, tests at concentrations of 7.5% and 10% were performed in duplicate, and the rest were performed once.

TABLE 20
Final stiffness of heat-set gels with differing
levels of added β-lactoglobulin

| | | | |
|---|---|---|---|
| B-lac | | | |
| concentration (% | Stiffness (G′) | Stiffness (G′) | |
| w/w) | 90° C. (Pa) | 20° C. (Pa) | Appearance |

Wild type β-lactoglobulin A

| | | | |
|---|---|---|---|
| 5% | 96 | 566 | opaque |
| 6.25% | 213 | 1276 | opaque |
| 7.5% | 474 | 2786 | opaque |
| 8.75% | 872 | 5259 | opaque |
| 10% | 1477 | 8875 | opaque |

β-lactoglobulin A PP29-30

| | | | |
|---|---|---|---|
| 5% | 62 | 152 | transparent |
| 6.25% | 208 | 642 | transparent |
| 7.5% | 691 | 1964 | transparent |
| 8.75% | 1685 | 4420 | transparent |
| 10% | 3193 | 8069 | transparent |

β-lactoglobulin A 007A

| | | | |
|---|---|---|---|
| 5% | 44 | 122 | transparent |
| 6.25% | 119 | 387 | transparent |
| 7.5% | 645 | 1829 | transparent |
| 8.75% | 1917 | 5061 | transparent |
| 10% | 3746 | 9909 | transparent |

β-lactoglobulin A 006B

| | | | |
|---|---|---|---|
| 5% | 106 | 327 | transparent |
| 6.25% | 397 | 1166 | transparent |
| 7.5% | 1315 | 3499 | transparent |
| 8.75% | 2783 | 7225 | transparent |
| 10% | 5229 | 13670 | transparent |

β-lactoglobulin 009D

| | | | |
|---|---|---|---|
| 5% | 37 | 177 | opaque |
| 6.25% | 420 | 1909 | opaque |
| 7.5% | 1157 | 4890 | opaque |
| 8.75% | 2528 | 11110 | opaque |
| 10% | 3946 | 17010 | opaque |

Wild type β-lactoglobulin B

| | | | |
|---|---|---|---|
| 5% | 151.8 | 726.95 | opaque |
| 6.25% | 379.6 | 1855 | opaque |
| 7.5% | 919.5 | 4715 | opaque |
| 8.75% | 1827 | 9441 | opaque |
| 10% | 2937 | 15050 | opaque |

β-lactoglobulin B PP26

| | | | |
|---|---|---|---|
| 5% | 508.6 | 1522 | transparent |
| 6.25% | 1200 | 3413 | transparent |
| 7.5% | 3139.5 | 9092 | transparent |
| 8.75% | 5614 | 16390 | transparent |
| 10% | 9574.5 | 28885 | transparent |

### Example 16

This example investigates the performance of protein compositions of the invention in the preparation of acid-set gels.

### 1. Preparation of Acid-Set Gels

The recombinant β-lactoglobulin protein compositions were as described in Example 12. Acid-set gels were prepared and their properties measured as described in Example 5.

### 2. Results

The results are presented in Table 21. In general, the recombinant β-lactoglobulin samples produced stiffer gels than in Example 5. The samples of β-lactoglobulin used in Example 5 had high levels of polysaccharide present, which likely interfered with the acid gelation process, resulting in weaker gels. The samples of β-lactoglobulin used in this Example were highly purified.

TABLE 21
Acid gelation properties of milk with differing levels of added β-lactoglobulin.

| | | | |
|---|---|---|---|
| Sample | Gelation time (min) | Gelation pH | Final stiffness (Pa) |

β-lactoglobulin A PP29-30

| | | | |
|---|---|---|---|
| milk | 26.5 | 5.35 | 267 |
| milk + 0.25% β-lac | 23.2 | 5.42 | 311 |
| milk + 0.5% β-lac | 22.2 | 5.44 | 298 |
| milk + 0.75% β-lac | 21.9 | 5.44 | 340 |
| milk + 1.0% β-lac | 19.5 | 5.49 | 547 |

β-lactoglobulin A 007A

| | | | |
|---|---|---|---|
| milk | 26.5 | 5.35 | 267 |
| milk + 0.25% β-lac | 23.2 | 5.42 | 276 |
| milk + 0.5% β-lac | 23.0 | 5.42 | 280 |
| milk + 0.75% β-lac | 20.9 | 5.46 | 322 |
| milk + 1.0% β-lac | 20.5 | 5.47 | 486 |

β-lactoglobulin A 006B

| | | | |
|---|---|---|---|
| milk | 26.5 | 5.35 | 267 |
| milk + 0.25% β-lac | 22.9 | 5.42 | 333 |
| milk + 0.5% β-lac | 22.2 | 5.44 | 334 |
| milk + 0.75% β-lac | 19.35 | 5.49 | 550 |
| milk + 1.0% β-lac | 18.7 | 5.51 | 862 |

β-lactoglobulin A 009D

| | | | |
|---|---|---|---|
| milk | 26.5 | 5.35 | 267 |
| milk + 0.25% β-lac | 23.5 | 5.41 | 270 |
| milk + 0.5% β-lac | 24.7 | 5.39 | 272 |
| milk + 0.75% β-lac | 23.9 | 5.40 | 268 |
| milk + 1.0% β-lac | 23.9 | 5.40 | 368 |

β-lactoglobulin B PP26

| | | | |
|---|---|---|---|
| milk | 26.5 | 5.35 | 267 |
| milk + 0.25% β-lac | 21.5 | 5.45 | 334 |
| milk + 0.5% β-lac | 20.3 | 5.48 | 369 |
| milk + 0.75% β-lac | 19.5 | 5.49 | 460 |
| milk + 1.0% β-lac | 16.5 | 5.55 | 623 |

### Example 17

This example investigates the acid-heat stability of protein compositions of the invention.

### 1. Preparation of Acidic Beverage Formulations

Model near-water acidified beverages with 2.1% protein were prepared according to NZMP's “Satiety Water with Whey Protein” Bulletin (AB.061; Version 2.0914). Stevia for sweetening, and lemon flavouring were omitted from the formulation.

The protein ingredients used were SureProtein™ WPI895 (a neutral pH WPI), SureProtein™ WPI8855 (an acidified WPI designed for acid beverages), and β-lactoglobulin A and B preparations PP29-30 and PP26 according to Example 12.

The protein was weighed into a beaker and 450 g water added. The solution was stirred for 60 minutes to reconstitute and hydrate the protein and then pH adjusted to 3.3 using 50% 1:1 citric:lactic acid for WPI895 and WPI8855, or 20% NaOH for PP29-30 and PP26. Water was added to bring the total to 500 g. The beverages were heat-treated on lab scale at 92° C./30 seconds and immediately cooled in iced water.

The processability of the beverages was assessed post heat-treatment. Absorbance at 610 nm as a measure of clarity was measured on day one, using water as the blank.

### 2. Results

All the beverages were processable and acid-heat stable. They showed no signs of floc formation or precipitation. The recombinant β-lactoglobulin beverage samples were transparent and straw-coloured. The WPI8855 acid beverage was more water-like in appearance, whilst the WPI895 solution was slightly turbid. Table 22 shows the absorbance at 610 nm, with an absorbance value of <0.1 being indicative of acceptable clarity.

TABLE 22
Absorbance values of 2.1% acidified,
heat-treated protein solutions.

| Protein | Absorbance at 610 nm |
|---|---|
| Water | 0.002 |
| WPI895 | 0.085 |
| WPI8855 | 0.032 |
| β-lg A (PP29-30) | 0.018 |
| β-lg B (PP26) | 0.017 |

On standing overnight, and after one week at ambient, there was no change in appearance and no signs of sediment formation in any of the samples.

It is not the intention to limit the scope of the invention to the abovementioned examples only. As would be appreciated by a skilled person in the art, many variations are possible without departing from the scope of the invention.

### EMBODIMENTS

The following numbered paragraphs define some particular embodiments of the present disclosure:

1. A composition comprising a plurality of recombinant β-lactoglobulin proteins heterogeneous in amino acid sequence, the recombinant proteins having at least 70% sequence identity to a wild type, mature β-lactoglobulin, wherein the plurality comprises at least one modified β-lactoglobulin protein comprising or consisting of an amino acid sequence that comprises
a) an N-terminal truncation of from about 1 to about 20 amino acids relative to the sequence of a wild type, mature β-lactoglobulin;
b) an N-terminal elongation of from about 1 to about 20 amino acids relative to the sequence of a wild type, mature β-lactoglobulin; or
c) one or more substitutions of from about 1 to about 20 amino acids relative to the sequence of a wild type, mature β-lactoglobulin,
   and wherein
A) the amount of modified β-lactoglobulin protein is at least 40% of the total recombinant β-lactoglobulin proteins in the composition;
B) the amount of modified β-lactoglobulin protein comprising or consisting of an amino acid sequence that comprises an N-terminal elongation of from about 1 to about 20 amino acids relative to the sequence of a wild type, mature β-lactoglobulin, is at least about 10% of the total recombinant β-lactoglobulin proteins in the composition;
C) the amount of modified β-lactoglobulin protein comprising or consisting of an amino acid sequence that comprises an N-terminal truncation of from about 1 to about 20 amino acids relative to the sequence of a wild type, mature β-lactoglobulin, is at least about 30% of the total recombinant β-lactoglobulin proteins in the composition; or
D) any combination of any two or more of (A) to (C).

2. The composition of embodiment 1, wherein the wild type, mature β-lactoglobulin has a sequence of one of SEQ ID Nos: 1-3, 34, 36, 38, 40, 42, 44, 45, or 47.

3. The composition of embodiment 2, wherein the wild type, mature β-lactoglobulin has a sequence of one of SEQ ID Nos: 1-3.

4. The composition of any one of embodiments 1 to 3, wherein the amount of recombinant β-lactoglobulin protein having an amino acid sequence comprising an aspartic acid at a position equivalent to position 80 of SEQ ID No: 1 and a valine at a position equivalent to position 134 of SEQ ID No: 1, is at least 90% of the total recombinant β-lactoglobulin protein in the composition.

5. The composition of any one of embodiments 1 to 4, wherein the amount of recombinant β-lactoglobulin protein having an amino acid sequence comprising a glycine at a position equivalent to position 80 of SEQ ID No: 2 and an alanine at a position equivalent to position 134 of SEQ ID No: 2, is at least 90% of the total recombinant β-lactoglobulin proteins in the composition.

6. A composition comprising a plurality of recombinant β-lactoglobulin proteins heterogeneous in amino acid sequence, the recombinant proteins having at least 70% sequence identity to SEQ ID No: 1, wherein the plurality comprises at least one modified β-lactoglobulin protein comprising or consisting of an amino acid sequence that comprises:
a) an N-terminal truncation of from about 1 to about 20 amino acids relative to the sequence of SEQ ID No: 1;
b) an N-terminal elongation of from about 1 to about 20 amino acids relative to the sequence of SEQ ID No: 1; or
c) one or more substitutions of from about 1 to about 20 amino acids relative to the sequence of SEQ ID No: 1,
   and wherein
A) the amount of modified β-lactoglobulin protein is at least 40% of the total recombinant β-lactoglobulin proteins in the composition;
B) the amount of modified β-lactoglobulin protein comprising or consisting of an amino acid sequence that comprises an N-terminal elongation of from about 1 to about 20 amino acids relative to the sequence of SEQ ID No: 1, is at least about 10% of the total recombinant β-lactoglobulin proteins in the composition;
C) the amount of modified β-lactoglobulin protein comprising or consisting of an amino acid sequence that comprises an N-terminal truncation of from about 1 to about 20 amino acids relative to the sequence of SEQ ID No: 1, is at least about 30% of the total recombinant β-lactoglobulin proteins in the composition; or
D) any combination of any two or more of (A) to (C).

7. A composition comprising a plurality of recombinant β-lactoglobulin proteins heterogeneous in amino acid sequence, the recombinant proteins having at least 70% sequence identity to SEQ ID No: 2, wherein the plurality comprises at least one modified β-lactoglobulin protein comprising or consisting of an amino acid sequence that comprises:
a) an N-terminal truncation of from about 1 to about 20 amino acids relative to the sequence of SEQ ID No: 2;
b) an N-terminal elongation of from about 1 to about 20 amino acids relative to the sequence of SEQ ID No: 2; or
c) one or more substitutions of from about 1 to about 20 amino acids relative to the sequence of SEQ ID No: 2,
   and wherein
A) the amount of modified β-lactoglobulin protein is at least 40% of the total recombinant β-lactoglobulin proteins in the composition;
B) the amount of modified β-lactoglobulin protein comprising or consisting of an amino acid sequence that comprises an N-terminal elongation of from about 1 to about 20 amino acids relative to the sequence of SEQ ID No: 2, is at least about 10% of the total recombinant β-lactoglobulin proteins in the composition;
C) the amount of modified β-lactoglobulin protein comprising or consisting of an amino acid sequence that comprises an N-terminal truncation of from about 1 to about 20 amino acids relative to the sequence of SEQ ID No: 2, is at least about 30% of the total recombinant β-lactoglobulin proteins in the composition; or
D) any combination of any two or more of (A) to (C).

8. The composition of any one of embodiments 1 to 7, wherein the recombinant β-lactoglobulin proteins have at least 75%, 80%, 85%, 90% or 95% sequence identity to
a) a wild type, mature β-lactoglobulin;
b) any one of SEQ ID Nos: 1-3, 34, 36, 38, 40, 42, 44, 45, or 47;
c) any one of SEQ ID Nos:1-3;
d) SEQ ID No:1 or 2;
e) SEQ ID No:1; or
f) SEQ ID No:2.

9. The composition of any one of embodiments 1 to 8, wherein the amount of modified β-lactoglobulin protein is at least 40, 45, 50, 55, 60, 65, 70 or 75% of the total recombinant β-lactoglobulin proteins in the composition.

10. The composition of any one of embodiments 1 to 9, wherein the plurality comprises at least one modified β-lactoglobulin protein comprising or consisting of an amino acid sequence that comprises one or more substitutions of from about 1 to about 20, 1 to 10, 1 to 5, 1 to 4, 1 to 3 amino acids, or one amino acid, relative to the sequence of
a) a wild type, mature β-lactoglobulin;
b) any one of SEQ ID Nos: 1-3, 34, 36, 38, 40, 42, 44, 45, or 47;
c) any one of SEQ ID Nos:1-3;
d) SEQ ID No:1 or 2;
e) SEQ ID No:1; or
f) SEQ ID No:2.

11. The composition of any one of embodiments 1 to 10, wherein the plurality comprises at least one modified β-lactoglobulin protein comprising or consisting of an amino acid sequence that comprises an N-terminal elongation of from about 1 to 15, about 1 to 10 or about 1 to 5 amino acids

12. The composition of any one of embodiments 1 to 11, wherein the plurality comprises at least one modified β-lactoglobulin protein comprising or consisting of an amino acid sequence that comprises an N-terminal truncation of from about 1 to 15, about 1 to 10 or about 1 to 5 amino acids.

13. The composition of any one of embodiments 1 to 12, wherein the plurality comprises at least one modified β-lactoglobulin protein comprising or consisting of an amino acid sequence that comprises one or more substitutions of from about 1 to 10, 1 to 5, 1 to 4, or 1 to 3 amino acids, or one amino acid relative to the sequence of one of SEQ ID Nos: 1 to 3.

14. The composition of any one of embodiments 1 to 13, wherein the plurality comprises at least one modified β-lactoglobulin protein comprising or consisting of an amino acid sequence that comprises one or more substitutions of from about 1 to about 20 amino acids relative to the sequence of a wild type, mature β-lactoglobulin, and wherein the one or more substitutions comprises one or more essential amino acids.

15. The composition of embodiment 14, wherein the one or more essential amino acids comprise or consist of one or more histidine residues.

16. The composition of any one of embodiments 1 to 15, wherein:
a) the amount of recombinant β-lactoglobulin protein consisting of an amino acid sequence of any one of SEQ ID Nos 1 to 3, is from about 1 to about 10% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of any one of SEQ ID Nos 1 to 3, is about 50 to about 99% of the total recombinant β-lactoglobulin proteins in the composition; and
c) the amount of modified β-lactoglobulin protein comprising or consisting of an amino acid sequence that comprises an N-terminal truncation of from about 1 to about 20 amino acids relative to the sequence of any one of SEQ ID Nos 1 to 3, is at least about 30% of the total recombinant β-lactoglobulin proteins in the composition.

17. The composition of any one of embodiments 1 to 15, wherein:
a) the amount of recombinant β-lactoglobulin protein consisting of an amino acid sequence of any one of SEQ ID Nos 1 to 3, is from about 1 to about 50% of the total recombinant β-lactoglobulin proteins in the composition;
b) the amount of modified β-lactoglobulin protein comprising or consisting of an amino acid sequence that comprises an N-terminal elongation of from about 1 to about 20 amino acids relative to the sequence of any one of SEQ ID Nos 1 to 3, is about 0 to about 10% of the total recombinant β-lactoglobulin proteins in the composition; and
c) the amount of modified β-lactoglobulin protein comprising or consisting of an amino acid sequence that comprises an N-terminal truncation of from about 1 to about 20 amino acids relative to the sequence of any one of SEQ ID Nos 1 to 3, is from about 50 to about 99% of the total recombinant β-lactoglobulin proteins in the composition.

18. The composition of any one of embodiments 1 to 17, wherein the modified β-lactoglobulin protein comprises one or more recombinant proteins each comprising or consisting of a sequence of any one of SEQ ID Nos: 4-7 and SEQ ID Nos: 19-22.

19. The composition of any one of claims 1 to 18, wherein the modified β-lactoglobulin protein comprises one or more recombinant proteins each comprising or consisting of a sequence of any one of SEQ ID Nos: 8-18 and SEQ ID Nos: 23-30.

20. The composition of any one of embodiments 1 to 19, wherein the plurality of recombinant β-lactoglobulin proteins has a secondary protein structure comprising or consisting of:
a) from about 0 to about 6% α-helix;
b) from about 40 to about 55% anti-parallel β-sheet;
c) from about 0 to about 3% parallel β-sheet;
d) from about 8 to about 20% turns; and
e) about 35 to about 45% unspecified or unordered structure.

21. The composition of any one of embodiments 1 to 20, wherein the plurality of recombinant β-lactoglobulin proteins has a mean DIAAS score of at least 0.8.

22. The composition of any one of embodiments 1 to 21, wherein the plurality of recombinant β-lactoglobulin proteins denatures at a higher temperature than a protein consisting of the sequence of SEQ ID No:1 or 2 as measured by differential scanning calorimetry.

23. The composition of any one of embodiments 1 to 21, wherein the plurality of recombinant β-lactoglobulin proteins denatures at a lower temperature than a protein consisting of the sequence of SEQ ID No:1 or 2 as measured by differential scanning calorimetry.

24. The composition of any one of embodiments 1 to 23, wherein the storage modulus of a heat set gel comprising the composition at a pH of about 7 is at least equivalent to that of NZMP SureProtein™ WPI895 when measured at a frequency of 1 Hz and at 20° C., wherein the heat set gel is formed by heating a solution comprising 10% by weight of the plurality of recombinant β-lactoglobulin proteins or WPI895 by heating from 20 to 80° C. at a rate of 1° C./min, holding the solution at 80° C. for 30 min, and cooling the solution from 80 to 20° C. at a rate of 1° C./min and holding the temperature at 20° C. for 20 min on a rheometer to form the gel.

25. The composition of any one of embodiments 1 to 24, wherein a shelf-stable protein beverage comprising 3.5% by weight of the plurality of recombinant β-lactoglobulin proteins exhibits no visible sedimentation after 4 weeks at ambient temperature.

26. The composition of any one of embodiments 1 to 25, wherein a solution having a pH of about 7 and comprising 10% by weight of the plurality of recombinant β-lactoglobulin proteins has an overrun of at least 500% after 10 minutes whipping, and/or wherein a foam produced by whipping a solution of neutral pH comprising 10% by weight of the plurality of recombinant proteins exhibits no serum leakage for at least about 5 minutes after whipping.

27. The composition of any one of embodiments 1 to 26, wherein an emulsion comprising the composition exhibits increased, the same or reduced stability compared with an emulsion comprising a protein having a sequence of SEQ ID Nos:1 or 2, wherein the emulsion is formed by combining the composition with oil in an amount of from 2.5 to 17.5% and water, pre-homogenizing and homogenizing by sonication at 100% amplitude for 10 min, and measuring particle size using laser light scattering.

28. The composition of any one of embodiments 1 to 27, wherein the emulsion activity index (EAI) of an emulsion comprising the composition is increased, the same or decreased compared with an emulsion comprising a protein having a sequence of any one of SEQ ID Nos:1 to 3, wherein the emulsion is formed by preparing a solution comprising 0.5% of the plurality of recombinant β-lactoglobulin proteins, 20% soya oil and water and homogenizing the solution at 200 bar, and wherein the EAI is determined by measuring the specific surface area of the emulsion droplets using laser light scattering.

29. The composition of any one of embodiments 1 to 28, wherein the composition is substantially free of aspartyl protease-like activity.

30. A food product comprising a composition of any one of embodiments 1 to 29.

31. The food product of embodiment 30 wherein the food product is in bar or solid moulded form.

32. A method for preparing a food product, the method comprising
a) providing a composition of any one of embodiments 1 to 29; and
b) mixing the composition with one or more additional ingredients to produce the food product.

33. The food product of embodiment 30 or 31 or the method of embodiment 32, wherein the food product is selected from the group comprising a fermented food, a yoghurt, a soup, a sauce, a bar, a gel, a foam, a nutritional formulation, a beverage, a beverage whitener, a cheese, a dairy tofu, a food emulsion and a dessert.

34. The product or the method of embodiment 33, wherein the nutritional formulation or food is an infant formula, toddler milk, growing up formula, maternal formula, a sports beverage, food for active lifestyles, a medical food or supplement.

35. The product or the method of embodiment 33, wherein the beverage is selected from the group comprising a dairy beverage, a sports beverage, a smoothie, a protein fortified fruit or vegetable juice, a drinking yoghurt, an acid protein fortified beverage, a jelly drink, protein water, liquid coffee, liquid tea, and a liquid beverage whitener.

36. The product or method of any one of embodiments 30 to 35, wherein the food product comprises an additional source of protein.

37. The product or method of embodiment 36, wherein the additional source of protein is a non-dairy source.

38. The product or method of embodiment 37, wherein the non-dairy source comprises a plant source, algal protein, mycoprotein, or a combination thereof.

39. The product or method of embodiment 38, wherein the plant source comprises one or more legumes, grains, seeds, nuts, tubers, or a combination thereof.

40. The product or method of embodiment 39, wherein
a) the legumes comprise soy, pea, lentil, chickpea, peanut, bean or a combination thereof;
b) the grains comprise wheat, rice, oat, corn or a combination thereof;
c) the seeds comprise canola, flaxseed (linseed), hemp, sunflower, quinoa, chia, or a combination thereof;
d) the nuts comprise almond, cashew, walnut or a combination thereof; and/or
e) the tuber comprises potato.

41. The product or method of any one of embodiments 30 to 40, wherein the food product is suitable for those on a vegan diet.

42. The product of method of any one of embodiments 30 to 41, wherein the food product comprises casein, preferably κ-casein.

43. A polynucleotide expression vector for expressing a plurality of recombinant β-lactoglobulin proteins heterogeneous in amino acid sequence, the polynucleotide expression vector encoding
a) a signal sequence;
b) a leader sequence;
c) a β-lactoglobulin protein; and
d) a processing site selected from KR, KREA, KREAEA and KREAEAM located between the leader sequence and the mature dairy protein.

44. A host cell comprising the expression vector of embodiment 43.

45. A host cell for expressing a plurality of recombinant β-lactoglobulin proteins heterogeneous in amino acid sequence, wherein the species of the host cell is selected from Pichia pastoris, Kluyveromyces lactis, Saccharomyces cerevisiae and Aspergillus niger, and wherein the host cell comprises a polynucleotide encoding
a) a signal sequence;
b) a leader sequence;
c) a β-lactoglobulin protein; and
d) a processing site selected from KR, KREA, KREAEA and KREAEAM located between the leader sequence and the β-lactoglobulin protein.

46. The polynucleotide expression vector of embodiment 43 or the host cell of embodiment 44 or 45, wherein the polynucleotide encodes a β-lactoglobulin protein comprising or consisting of the sequence of any one of SEQ ID Nos:1 to 3.

47. The host cell of any one of embodiments 44 to 46, wherein the host cell lacks an operative pep4 gene, or has been modified to produce less PEP4 protein than a wild type control cell.

48. A method for producing a plurality of recombinant β-lactoglobulin proteins heterogeneous in amino acid sequence, comprising
a) culturing a host cell of any one of embodiments 43 to 46 in a culture medium under conditions sufficient to allow for expression of the one or more recombinant β-lactoglobulin proteins; and
b) isolating the one or more recombinant β-lactoglobulin proteins from the culture medium.

49. The polynucleotide expression vector, host cell or method according to any one of embodiments 43 to 48, wherein the recombinant proteins have at least 70% sequence identity to any one of SEQ ID Nos: 1-3.

1. A composition comprising two or more recombinant β-lactoglobulin proteins of differing amino acid sequence, the recombinant β-lactoglobulin proteins having at least 70% sequence identity to a wild type, mature β-lactoglobulin, and wherein at least one of the β-lactoglobulin proteins is an elongated β-lactoglobulin protein.

2. The composition of claim 1 wherein the elongated β-lactoglobulin protein has an amino acid sequence comprising or consisting of
a) a core sequence having at least 70% sequence identity to the sequence of a wild type, mature β-lactoglobulin; and
b) an N-terminal elongation of the core sequence, the N-terminal elongation consisting of from about 1 to about 20 amino acids.

3. The composition of claim 1 or 2, wherein the composition comprises one or more recombinant, elongated β-lactoglobulin proteins having an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of 1 amino acid or 2 or more contiguous amino acids from the sequence KREAEAM, KREAEAEAM, or KREAEAEAEAM.

4. The composition of claim 2 or 3, wherein the N-terminal elongation has a sequence comprising or consisting of 1 amino acid or 2 or more contiguous amino acids from the sequence

| | |
|---|---|
| a) | REAEAEAEAM, REAEAEAM, or REAEAM |
| b) | EAEAEAEAM, EAEAEAM, or EAEAM; |
| | or |
| c) | EAEAEAEA, EAEAEA, or EAEA. |

5. The composition of any one of claims 1 to 4, wherein the composition comprises one or more recombinant, elongated β-lactoglobulin proteins having an N-terminal elongation having a sequence comprising or consisting of any one of:
a) EAEAM
b) EAEA
c) EAM
d) EA
e) three or more repeats of EA
f) M; and
g) R.

6. The composition of any one of claims 1 to 5, wherein the composition comprises one or more recombinant, elongated β-lactoglobulin proteins comprising or consisting of a sequence of any one of SEQ ID Nos: 4-7, 19-22 and 71-74.

7. The composition of any one of claims 1 to 6, wherein the composition comprises two or more or three or more recombinant, elongated β-lactoglobulin proteins of differing amino acid sequence.

8. The composition of any one of claims 1 to 7, wherein the amount of recombinant, elongated β-lactoglobulin proteins in the composition is at least about 4% of total recombinant β-lactoglobulins in the composition.

9. The composition of claim 8, wherein the amount of recombinant, elongated β-lactoglobulin proteins in the composition is at least about 40%, 50% or 60% of total recombinant β-lactoglobulins in the composition.

10. The composition of any one of claims 1 to 9, wherein the composition further comprises
a) one or more recombinant, truncated β-lactoglobulin proteins that comprise or consist of an amino acid sequence having an N-terminal truncation of from about 1 to about 20 amino acids relative to the sequence of a wild type, mature β-lactoglobulin; and/or
b) one or more recombinant, substituted β-lactoglobulin proteins that comprise or consist of an amino acid sequence having one or more substitutions of from about 1 to about 20 amino acids relative to the sequence of a wild type, mature β-lactoglobulin.

11. The composition of any one of claims 1 to 11, wherein the amount of modified β-lactoglobulin proteins in the composition is at least 45%, 50% or 60% of the total recombinant β-lactoglobulin proteins in the composition.

12. The composition of claim 10 or 11, wherein the one or more substituted β-lactoglobulin proteins each comprise or consist of an amino acid sequence that comprises one or more substitutions of from about 1 to 10, 1 to 5, 1 to 4, 1 to 3 amino acids, or one amino acid, relative to the sequence of
a) a wild type, mature β-lactoglobulin;
b) any one of SEQ ID Nos: 1-3, 34, 36, 38, 40, 42, 44, 45, or 47;
c) any one of SEQ ID Nos:1-3;
d) SEQ ID No:1 or 2;
e) SEQ ID No:1; or
f) SEQ ID No:2.

13. The composition of any one of claims 10 to 12, wherein the one or more truncated β-lactoglobulin proteins each comprise or consist of a sequence of any one of SEQ ID Nos: 8-18 and SEQ ID Nos: 23-30.

14. The composition of any one of claims 1 to 13, wherein the composition is substantially free of aspartyl protease-like activity.

15. The composition of any one of claims 1 to 14, wherein the amount of recombinant β-lactoglobulin protein having an amino acid sequence comprising an aspartic acid at a position equivalent to position 80 of SEQ ID No: 1 and a valine at a position equivalent to position 134 of SEQ ID No: 1, is at least 90% of the total recombinant β-lactoglobulin protein in the composition.

16. The composition of any one of claims 1 to 14, wherein the amount of recombinant β-lactoglobulin protein having an amino acid sequence comprising a glycine at a position equivalent to position 80 of SEQ ID No: 2 and an alanine at a position equivalent to position 134 of SEQ ID No: 2, is at least 90% of the total recombinant β-lactoglobulin proteins in the composition.

17. A recombinant, elongated β-lactoglobulin protein having an amino acid sequence comprising or consisting of
a) a core sequence having at least 70% sequence identity to the sequence of a wild type, mature β-lactoglobulin; and
b) an N-terminal elongation of the core sequence, the N-terminal elongation having a sequence comprising or consisting of
i) 2 or more contiguous amino acids from the sequence KREAEAEAEAM, KREAEAEAM, or KREAEAM; or
ii) R.

18. The recombinant, elongated β-lactoglobulin protein of claim 17, wherein the N-terminal elongation has a sequence comprising or consisting of 2 or more contiguous amino acids from the sequence

| | |
|---|---|
| a) | REAEAEAEAM, REAEAEAM, or REAEAM |
| b) | EAEAEAEAM, EAEAEAM, or EAEAM; |
| | or |
| c) | EAEAEAEA , EAEAEA, or EAEA. |

19. The recombinant, elongated β-lactoglobulin protein of claim 17 or 18, wherein the N-terminal elongation has a sequence comprising or consisting of any one of:
a) EAEAM,
b) EAEA,
c) EAM,
d) EA;
e) three or more repeats of EA; and
f) R.

20. The recombinant, elongated β-lactoglobulin protein of any one of claims 17 to 19, wherein the recombinant, elongated β-lactoglobulin protein has an amino acid sequence comprising or consisting of a sequence of any one of SEQ ID Nos: 4, 5, 7, 19-20, 22 and 71-74.

21. A composition comprising one or more recombinant, elongated β-lactoglobulin proteins of any one of claims 17 to 20.

22. The composition of any one of claims 1 to 16 or 21, or the recombinant, elongated β-lactoglobulin protein of any one of claims 17 to 20, wherein the wild type, mature β-lactoglobulin has a sequence of any one of SEQ ID Nos: 1-3, 34, 36, 38, 40, 42, 44, 45, or 47.

23. The composition or recombinant, elongated β-lactoglobulin protein of claim 22, wherein the wild type, mature β-lactoglobulin has a sequence of any one of SEQ ID Nos: 1-3.

24. The composition or recombinant, elongated β-lactoglobulin protein of claim 22 or 23, wherein the one or more recombinant β-lactoglobulin proteins have at least 75%, 80%, 85%, 90% or 95% sequence identity to
a) a wild type, mature β-lactoglobulin;
b) any one of SEQ ID Nos: 1-3, 34, 36, 38, 40, 42, 44, 45, or 47;
c) any one of SEQ ID Nos:1-3;
d) SEQ ID No:1 or 2;
e) SEQ ID No:1; or
f) SEQ ID No:2.

25. A food product comprising a recombinant, elongated β-lactoglobulin protein having at least 70% sequence identity to a wild type, mature β-lactoglobulin.

26. A method for preparing a food product, the method comprising
a) providing a recombinant, elongated β-lactoglobulin protein having at least 70% sequence identity to a wild type, mature β-lactoglobulin, and
b) mixing the composition with one or more additional ingredients to produce the food product.

27. The food product of claim 25 or the method of claim 26, wherein the recombinant, elongated β-lactoglobulin protein comprises a protein or composition of any one of claims 1 to 24.

28. The food product or method of any one of claims 25 to 27, wherein the food product is in bar or solid moulded form.

29. The food product or method of any one of claims 25 to 27, wherein the food product is selected from the group comprising a fermented food, a yoghurt, a soup, a sauce, a bar, a gel, a foam, a nutritional formulation, a beverage, a beverage whitener, a cheese, a dairy tofu, a food emulsion and a dessert.

30. The food product or the method of claim 29, wherein the nutritional formulation or food is an infant formula, toddler milk, growing up formula, maternal formula, a sports beverage, food for active lifestyles, a medical food or supplement.

31. The food product or the method of claim 30, wherein the beverage is selected from the group comprising a dairy beverage, a sports beverage, a smoothie, a protein fortified fruit or vegetable juice, a drinking yoghurt, an acid protein fortified beverage, a jelly drink, protein water, liquid coffee, liquid tea, and a liquid beverage whitener.

32. The food product or method of any one of claims 25 to 31, wherein the food product comprises an additional source of protein.

33. The food product or method of claim 32, wherein the additional source of protein is a non-dairy source.

34. The food product or method of claim 33, wherein the non-dairy source comprises a plant source, algal protein, mycoprotein, or a combination thereof.

35. The food product or method of claim 34, wherein the plant source comprises one or more legumes, grains, seeds, nuts, tubers, or a combination thereof.

36. The food product or method of claim 35, wherein
a) the legumes comprise soy, pea, lentil, chickpea, peanut, bean or a combination thereof;
b) the grains comprise wheat, rice, oat, corn or a combination thereof;
c) the seeds comprise canola, flaxseed (linseed), hemp, sunflower, quinoa, chia, or a combination thereof;
d) the nuts comprise almond, cashew, walnut or a combination thereof; and/or
e) the tuber comprises potato.

37. The food product or method of any one of claims 25 to 36, wherein the food product is suitable for those on a vegan diet.

38. The food product or method of any one of claims 25 to 37, wherein the food product does not comprise any milk proteins other than the recombinant, β-lactoglobulin proteins.

39. The food product of method of any one of claims 25 to 37, wherein the food product comprises casein, preferably κ-casein.

40. A polynucleotide expression vector for expressing a composition or recombinant, elongated β-lactoglobulin of any one of claims 1 to 24, the polynucleotide expression vector encoding
a) a signal sequence;
b) a leader sequence;
c) a β-lactoglobulin protein; and
d) a processing site selected from KR, KREA, KREAEA, KREAEAEA, KREAEAEAEA, KREAEAM, KREAEAEAM, or KREAEAEAEAM located between the leader sequence and the mature dairy protein.

41. A host cell comprising the expression vector of claim 40.

42. A host cell for expressing a composition or recombinant, elongated β-lactoglobulin of any one of claims 1 to 24, wherein the species of the host cell is selected from Pichia pastoris, Kluyveromyces lactis, Saccharomyces cerevisiae and Aspergillus niger, and wherein the host cell comprises a polynucleotide encoding
a) a signal sequence;
b) a leader sequence;
c) a β-lactoglobulin protein; and
d) a processing site selected from KR, KREA, KREAEA, KREAEAEA, KREAEAEAEA, KREAEAM, KREAEAEAM, or KREAEAEAEAM located between the leader sequence and the β-lactoglobulin protein.

43. The polynucleotide expression vector of claim 40 or the host cell of claim 41 or 42, wherein the polynucleotide encodes a β-lactoglobulin protein comprising or consisting of the sequence of any one of SEQ ID Nos:1 to 3.

44. The host cell of any one of claims 41 to 43, wherein the host cell lacks an operative pep4 gene, or has been modified to produce less PEP4 protein than a wild type control cell.

45. The polynucleotide expression vector or the host cell of any one of claims 40 to 44 wherein the processing site is selected from KREA, KREAEA and KREAEAM.

46. A method for producing a composition or recombinant, elongated β-lactoglobulin of any one of claims 1 to 24, the method comprising
a) culturing a host cell of any one of claims 41 to 45 in a culture medium under conditions sufficient to allow for expression of one or more recombinant β-lactoglobulin proteins; and
b) isolating the one or more recombinant β-lactoglobulin proteins from the culture medium.
